(19) United States
(12) Patent Application Publication
(10) Pub. No.: US 2023/0025694 A1
(43) Pub. Date: Jan. 26, 2023

(54) Silicone Hydrogel Based Fluorescent Assay and Contact Lens

(71) Applicant: University Of Maryland, Baltimore, Baltimore, MD (US)

(72) Inventors: Joseph R. Lakowicz, Ellicott City, MD (US); Ramachandram Badugu, Ellicott City, MD (US); E. Albert Reece, Lutherville, MD (US)

(21) Appl. No.: 17/752,733

(22) Filed: May 24, 2022

Related U.S. Application Data
(63) Continuation-in-part of application No. 16/319,179, filed on Jan. 18, 2019, now Pat. No. 11,369,295.
(60) Provisional application No. 62/451,824, filed on Jan. 30, 2017.
(60) Provisional application No. 62/364,444, filed on Jul. 20, 2016.
(60) Provisional application No. 63/196,753, filed on Jun. 4, 2021.

(51) Int. Cl. A61B 5/00; G01N 33/543; A61B 5/145; A61B 5/1455; C09K 11/06; C07F 5/02; G02B 1/04; G02C 7/04

(52) U.S. Cl. CPC A61B 5/6821; G01N 33/54373; A61B 5/14546; A61B 5/14507; A61B 5/14539; A61B 5/1455; C09K 11/06; C07F 5/025; G02B 1/043; G02C 7/04; C09K 2211/1022; C09K 2211/1007; C09K 2211/1014; C09K 2211/1011

(57) ABSTRACT

A material, article, system and method include a probe composition that includes a hydrophobic portion, a hydrophilic portion, an analyte-binding portion and a fluorophore portion. The analyte-binding portion is configured to bind to an analyte in an aqueous solution. The fluorophore portion is configured to change an optical property of fluorescent light emitted in response to incident excitation light when the probe composition changes between a first state in which the analyte is not bound to the analyte-binding portion and a second state in which the analyte binds to the analyte-binding portion. A material includes the probe composition and a silicone hydrogel substrate having a hydrogel network that allows flow of aqueous solution through the solution and a silicone network that occupies interstices of the hydrogel network. A contact lens having the material enables remote detection of glucose concentration in tear fluid of a subject.

### CROSS-REFERENCE TO RELATED APPLICATIONS

This application is a continuation-in-part of allowed U.S. patent application Ser. No. 16/319,179, filed 18 Jan. 2019, which is a national stage entry of International Patent Application No. PCT/US2017/043087, filed 20 Jul. 2017, which claims the benefit of U.S. provisional application Ser. No. 61/451,824, filed 30 Jan. 2017 and U.S. provisional application Ser. No. 62/364,444, filed 20 Jul. 2016. This application also claims the benefit of U.S. provisional patent application Ser. No. 63/196,753, filed 4 Jun. 2021. The entire contents of each of these applications are hereby incorporated by reference as if fully set forth herein.

### GOVERNMENT FUNDING SUPPORT

This invention was made with government support under grant nos. GM129561 and GM125976 awarded by the National Institutes of Health. The government has certain rights in the invention.

### BACKGROUND

### 1. Field of the Invention

The invention relates to the general field of clinical assays in medicine. In particular, this specification discloses clinical assays for tear fluid incorporating a silicon hydrogel contact lens.

### 2. Background of the Invention

For some conditions, such as dry eye, keratitis and diabetes, it is useful to detect the presence or concentration of analytes, such as electrolytes, biomolecules, or glucose, in tear fluid. However, at the present time there are limited practical ways to measure tear analytes because the irritation caused by sample collection disturbs the stationary analyte concentrations in tear fluid. Current in situ methods to measure these analytes, including using a contact lens with an electronic sensor come with costs involving instability, inefficiency, complexity or expense, or some combination.

### Anatomy and Physiology of the Cornea and Tear Layer

The cornea is about 550 μm thick and contains several distinct layers. The stroma consists of collagenase proteins and 70-80% water and is about 500 μm thick. The epithelium consists of about 5 layers of cells with thickness of 50 μm. The tear fluids form layered films. The reported thickness of these films varies greatly depending on the measurement method. The interferometry method can provide an average thickness, but a more recent method is high resolution optical coherence tomography (OCT). This method has been used to measure thickness and to provide cross-sectional images.

The tear film provides protection of the epithelial cells. The tear film is constantly replaced by new tears released from the tear ducts and Meibomian glands. This secretion spontaneously forms layers. The multi-layer film forms a complex three layer structure which consists of a hydrophilic layer adjacent to the corneal cells, a central aqueous region containing electrolytes and proteins, and a hydrophobic outer layer which slows water evaporation.

The lower hydrophilic layer is in direct contact with the epithelial cells and is highly permeable to ions. The electrolytes must be kept with a narrow range of concentration to keep the epithelial cells healthy, and the ion concentrations are carefully controlled to maintain the corneal cells. This layer also contains mucins and a glycocalyx layer. The tear film (TF) middle layer is aqueous and contains a large number of proteins and electrolytes. The top hydrophobic layer is formed with lipids to reduce evaporation of the middle aqueous layer. But evaporation cannot be avoided, which is why humans blink about 15 to 20 times per minute, refreshing the tear film to clean and maintain a continuous layer. The middle aqueous layer contains electrolytes and many proteins which include antibodies and lysozyme to disrupt bacterial cells (see FIG. 1). The tears are continually replaced by secretions from the lachrimal glands at a rate of 35% replacement per minute, and the Meibomian glands which secrete lipid. The tear film forms spontaneously from these secretions.

When a contact lens is inserted on the eye it becomes localized in the central aqueous layer and is directly exposed to the tear fluid. This process creates two new layers of tear films, the pre-lens tear film (PLTF) under the outer hydrophobic layer and the post-lens tear film between the inner surfaces of the contact lens and direct contact with the corneal cells. See FIG. 1. Because the corneal cells are active in ion transport it is easy to imagine the concentrations can be different in the PLTF and PoLTF, especially if the lenses are not penneable to ions.

The outer layer of the tear fluids is the pre-corneal lens film (PCLF). The precise thickness is uncertain but the consensus value is about 3 μm. The PCFT contains lipids which slow evaporation. A dramatic reorganization of the tear film occurs with placement of a contact lens, however. The lens is said to be in the tear layer, not on the tear layer. The anterior surface of the contact lens is covered by the pre-lens tear film (PLTF) which is rich in lipids. The posterior surface of the lens is coated with the post-lens tear film (PoLTF) which is on top of the cornea (FIG. 2), and in direct contact with the corneal epithelium. The reported thickness of these layers is variable (about 2-4 μm for the PLFT and about 3-8 μm for the PoLFT thickness. The contact lenses thickness depends on the degree of corrections, but generally are much thicker than the PLTF and PolTF and range from 70-200 μm in the center and become thicker away from the center. See FIG. 1 and FIG. 2 for schematic diagrams.

It is very difficult to obtain samples of bulk tears because of the small volume in each eye (about 7 μL). The eyes respond quickly to any physical contact which changes the electrolyte composition. Additionally, a bulk tear sample may not represent the PolTF or PLTF. Any attempt to obtain a sample either tear film will change the tear composition and result in mixing of the tear film with the bulk tear fluid. The measurement of pH in tear films is even more difficult because of the much lower concentration of H⁺ compared to the Na● and Cl⁻ (see Table 1). At present only total electrolyte concentration in bulk tears are measured by conductivity. The sample is obtained by very brief contact before the tear composition can change.

TABLE 1
Electrolyte Concentrations
in Blood Serum and Tears.

| Ions | Blood | | Tears | |
|---|---|---|---|---|
| pH | 7.35-7.46 | | 6.5-7.6 | |
| [H⁺] | 35-45 | nM | 25-316 | nM |
| Na⁺ | 135-148 | mM | 132 | mM |
| K⁺ | 3.5-5.3 | mM | 24 | mM |
| Ca⁺⁺ | 4.5-5.5 | mM | 0.8 | mM |
| Mg⁺⁺ | 0.7-1.0 | mM | 0.6 | Mm |
| Cl⁻ | 95-110 | mM | 118-138 | mM |

When an injury occurs, chloride is actively pumped through the epithelium into the PoTLF and sodium ions are actively transported into the stroma. Any disruption of the EL results in ionic currents and thus disruptions to the ion concentrations due to a large influx of chloride ions and efflux of calcium and potassium ions. The initial effect of corneal injury is the migration of activated neutrophils to the injured site. This results in a burst of respiration, and release of reactive oxygen species (ROS) and a decrease in pH. To avoid damage to nearby cells, the neutrophils need to be turned off, which is accomplished by an increase in pH. Simultaneously, there is a large efflux of chloride ions from the stroma into the tear film. See FIG. 3. The increased chloride concentration results in an electric potential towards the site of damage. In corneal wound healing and wound healing in other tissues, the electric field provides a dominant and over-riding signal that directs cell migration. The nearby epithelial cells migrate towards the electric field to rapidly fill in and repair the damage cornea. The electric field effect has been used to accelerate wound heating in other tissues. The decrease in pH is transient, but the chloride (potential) gradient can exist in cornea for up to 30 minutes. Ion-sensitive fluorophores on the posterior and/or anterior sides of contact lenses according to the invention can respond to these ion fluxes in the PolTF by changes in their fluorescence intensities, emission spectra, or lifetimes.

The PLTF and PolTF may have different ion concentrations. Free diffusion of ions such as Na can diffuse the 100 μm distance (thickness) of a contact lens in 5 seconds. The PoLTF volume of this phase is small (less than 1 μl) as compared to the 7 μl total volume of tears per eye. The contact lenses almost certainly are a barrier to rapid tear exchange. The PolTF is exchanged about 3-fold more slowly than the tears, about 15% per minute as compared to 44% per minute for bulk tears. These rates may explain why the incidence of keratitis has not decreased even with the many advances in contact lens polymers Blinking may increase the rate of exchange between the tear layers and bulk tears. The PolTF is exchanged more rapidly with hard lenses than soft lenses, which may explain to lower incidence of keratitis with hard lenses. These facts point towards the importance of measurements of the chemical composition of the PoLTF for research on the effects of contact lenses and for possible early detection of infections.

Any damage to of the tear film can result in infection of the cornea and stroma. The eyes are continuously exposed to the atmosphere and its impurities, but even with this continual exposure eye infections are rare. This resistance is due to the unique properties of the cornea and the tear film. The cornea is about 550 μm thick and composed of several layers (see FIG. 1). The stroma is about 500 μm thick and consist of collagenase tissue. About 10% of the stroma is keratocytes which replace the layers in the stroma. The stroma is covered by a thin layer of cells which are metabolically active and obtain oxygen directly from the air. These epithelial cells have tight junctions which prevent ion transport. These cells are constantly replaced and have a lifetime of 7 to 10 days. The epithelial layer of the cornea is a strong barrier against bacterial infections.

Keratitis is an example of some of the adverse effects that can occur with contact lens wear, especially extended wear lenses. Keratitis is an infection of the cornea, a condition which affects the cornea, resulting in inflammation and pain. Such infections can cause the cornea to become swollen with the appearance of cloudy patches or ulcers which can result in vision loss or blindness. Keratitis can be due to infection with bacteria, virus, amoeba, or fungus. Bacterial keratitis is the most common. Viral keratitis is known to be caused by HSV-1 and Zika, which can replicate in human cornea, but not the COVID-19 virus. Infections of the cornea have been rare but were reported more often following the introduction of soft contact lenses in the 1970s. The incidence of contact lens (CL) related microbial keratitis has remained stable for many years at 2-4 cases/per year 10,000 wearers per year for hard contact lenses and 20 per 10,000 wearers per year for soft CLs. Because of the large number of individuals wearing contact lenses (approximately 45 million in the USA as of 2018, and 140 million worldwide), even an infrequent complication results in many infections. The worldwide number of cases per year are 42,000 for hard lenses and 280,000 for soft lenses, respectively.

Corneal bacterial infections are most often due to Pseudomonas aeruginosa and several other bacteria, however despite the ubiquity of these bacteria, corneal infections are relatively rare. Epithelial cells grow rapidly and are sloughed off, which also helps to prevent infection by carrying away particles and bacteria. The epithelial layer (EL) is fragile and easily injured by rubbing the eyes, contact lens insertion and removal, and by minor and major injuries which can occur with physical contact. Small injuries are repaired rapidly and the cornea remains resistant to bacteria; deeper injuries can more often result in infections. The epithelial layer also is very active in ion transport.

Because keratitis is strongly associated with contact lens use, there has been an ongoing effort for nearly fifty years to improve the properties of contact lenses (CL) to reduce keratitis. One important development has been the introduction of silicone hydrogel lenses with increased rates of oxygen and electrolyte transport. Hydrogel (HG) and silicone hydrogel (SiHG) lenses were developed and approved for short term one-day wear or extended 15-30-day wear. Studies suggest that 50% of individuals who wear contact lenses report dryness and discomfort, which are the most common reasons for discontinuation of use. These results suggest that corneal changes and infections are the result of physical presence of the lens directly over the epithelial layer of the cornea, and not the chemical composition of the lenses.

Another fairly common result of tear defects is dry eye disease (DED). which affects 59 million patients in the United States. DED often disrupts the tear film, causing pain, loss of vision clarity and potentially, infection. The damaged regions can provide a place for entry of microbes and subsequent infection. DED can be the result of decreased tear secretion or increased tear evaporation, but in either event the result appears to be an increased electrolyte concentrations in tears. It seems reasonable to speculate that the presence of a physical barrier, the contact lens, can prolong ion-concentration changes caused in DED or due to corneal cell injury, and thereby contribute to the occurrence of keratitis.

Measurements of individual electrolyte concentrations are not reported in the literature because of the difficulties in sampling tear fluid. The only measurement in current use depends on brief touching of the eye to obtain a sample with basal ion levels. The fluid volume in a single eye is near 7 μl and the eyes respond quickly upon any contact, resulting in rapid increased secretion of the lacrimal gland and changes in ion concentrations in the collected fluid. The total electrolyte concentration is reported only as a bulk measurement of the total conductivity. The pH is almost never reported because of the low concentration of hydrogen ion (near 10⁻⁷ M) and the insignificant contribution of it to the bulk conductivity.

### Assays for Ophthalmic Study

In vivo confocal microscopy (ICM) has been used to study the cornea and to obtain some information when observing unlabeled epithelial cells. ICM is performed using backscattered light, and provides structural information but no information about the chemical composition of tear films. In general, fluorescent probes cannot be used in in vivo studies because human epithelial cells cannot be labeled in vivo for measurement.

Optical correlation tomography and interferometry (OCT) is widely used to obtain images of the cornea and measure the thickness of tear films. These images can detect structural changes in the epithelium and film thickness in response to contact lenses or other perturbations of the cornea, but cannot be used to determine the chemical composition of tear films.

Interferometry and reflectivity also are used to measure tear film thickness, but usually provide average values and not images. It can be difficult to distinguish the reflections from the multiple surfaces and there is no information on the tears or specificity of the chemical composition of the PoLTF.

Embodiments of the inventive methods here can be used to measure a single point on the cornea, or to obtain CL images of the entire contact lens with or without confocal optics. Non-confocal imaging is possible because the emission from the PLTF and PolTF can be separated based on the emission spectral properties. Fluorescence intensities or spectral imaging, or fluorescence lifetime imaging microscopy (FLIM) will allow ion concentration imaging of the entire contact lens.

### Evolution of Contact Lens Technology

Contact lenses were first described by Leonardo daVinci in 1508 and by Sir John Herscher in 1823, but a complete history is difficult to construct. Modern contact lenses were introduced in 1961 and the first full-scale commercial contact lens was introduced in 1971, These first commercial lenses were rigid and made of glass or poly(methyl methacrylate) (PMMA). While initially successful for improved vision, they were not suitable for long term wear because they are not permeable to oxygen and the corneal cells get their oxygen from the air.

The cornea gets oxygen from the air, and the increase in keratitis with hard lenses was thought to be due to lack of oxygen. Soft contact lenses were developed to improve comfort and allow oxygen transmission. The initial soft lenses were made of hydrogels (HG) made of various carbon-containing monomers such as cross-linked poly-hydroxy-methmethacrylate (HEMA). Oyxgen transmission improved but was still not adequate for continuous wear. The oxygen permeability of hydrogels is expressed by their Dk value and pure water has a Dk value near 80. Dk values below 22 are too low and result in cornea hypoxia. Dk values above 66 are suitable for daily or continuous wear. The Dk values of hydrogel polymers could be increased with less cross-linking, but the lenses became fragile and the Dk values could not be increased above that of pure water. The HG lenses were not recommended for continuous wear and definitely not while sleeping. It was well known that silicone was soft and highly permeable to oxygen. But silicone does not mix readily with water.

Silicon hydrogen (SiHG) lenses then were developed. These lenses provided dramatically increased oxygen transport. SiHG lenses are approved for extended 10- to 30-day wear even while sleeping. Presently, over 70% of new prescriptions are for some form of SiHG lens.

Unfortunately, the new softer HG and more permeable SiHG lenses did not result in a decreased incidence of keratitis. The keratitis severity appeared to be greater for extended wear SiHG lenses. Thus, the incidence does not appear to be linked to the chemical composition or oxygen permeabilities of the different lenses. The dependence of infection on wear time may be the result of contamination of the lenses or the containers by repeated handling of the lenses for extended wear.

It took about 20 years of research by multiple companies to develop optically clear silicone hydrogel (SiHG) lenses. These lenses are mostly silicon but contain a semi interpenetrating polymer network (INP) which consists of continuous channels across the lens for tear or water transport. See the SiHG lenses in Table 1, above. Oxygen transport occurs through the silicone regions, as shown in FIG. 4. This development to obtain continuous channels for oxygen rather than an emulsion-like structure with completely separate regions of silicone and water was remarkable.

In SiHG lenses, oxygen movement occurs through the continuous silicone regions and is even greater than for an equivalent thickness of water. Some of these lenses are surface-treated to make them more hydrophilic (polar). Because of the high oxygen transport, some SiHG lenses are approved for 30-day continuous wear, including sleeping with the lenses in place. Presently, there are numerous types of contact lenses on the market with slightly different polymers and surface treatments to reduce hydrophobicity. For some of the examples provided here, a second generation SiHG lens, Biofinity™ was selected. This lens uses the SiHG polymer Comfilicon A™, which contains a high silicon content near 40%. In other examples, the MyDay™ lenses, using Stenfilicon A™, were used.

The MyDay™ lenses were introduced in June 2015. They now are frequently prescribed. but only for one day use. These lenses have a very low silicone content (reported to be 4.4%), while maintaining high oxygen transmission (see Table 2, below). This remaining space in the MyDay™ lenses is probably a standard HG, but these details are not released by the company. In practice, the Biofinity™ and MyDay™ lenses are essentially the same in terms of comfort and absence of complications. HG lens Dailies (Nelficon A™) from Ciba Vision were chosen as a control.

### See Table 2.

TABLE 2
Selected Hydrogel and Silicone Hydrogel Contact Lenses.

| Polymer | Trade Name | Manufacturer | Water (%) | Dk |
|---|---|---|---|---|
| Lotrafilcon | Night and Day ™ | CIBA Vision | 23 | 140 |
| A (SiHG) | | | | |
| Galyfyilcon | Acuvue Advance ™ | Johnson & | 47 | 60 |
| A (SiHG) | | Johnson | | |
| Comfilcon | Biofinity ™ | Cooper | 48 | 128 |
| A (SiHG) | | Vision | | |
| Stenfilcon | MyDay ™ | Cooper | 54 | 80 |
| A (SiHG) | | Vision | | |
| Latrofilcon | Air OptixAqua ™ | CIBA Vision/ | 33 | 138 |
| B (SiHG) | | Alcon | | |
| Netfilcon A (HG) | Aqua Release ™ | CIBA Vision | 69 | 26 |
| Netfilcon A (HG) | Dailies ™ | CIBA Vision | 31 | 26 |

The SiHG lenses have high permeability to both oxygen and ionic species. These characteristics are due to a unique three-dimensional structure which consists of interpenetrating polymer networks (JPN). In the Biofinity™ lenses, there are continual pores from the front to the back of the lens, which allows rapid transport of ions. The MyDay™ lenses are claimed to have the opposite 3D structure, consisting of thin continuous silicon chains with the remaining value filled with a standard hydrogel and/or tear fluid. The reason for development of MyDay™ lenses was the residual hydrophobic properties of the Biofinity™ lenses with a high silicone content which resulted in eye irritation. Both lenses are optically clear, which indicates that the IPN network has sub-wavelength dimensions which do not scatter light. The pore size in the Biofinity™ type lenses is thought to be about 50 nm in diameter. The topology of the SiHG lenses provides opportunities for labeling which are not present with standard HG lenses. See FIG. 4. HG lenses are thought to have the polymer uniformly dispersed through the lens. This results in decreased opportunity for their use for sensing, especially with non-covalently bound fluorophores.

The Biofinity™ and MyDay™ lenses appear to have different chemical and physical properties (see FIG. 4). Biofinity™ lenses are based on the polymer Comfilcon A™, which is a 2nd generation SiHG and approved for extended wear up to 30 days, but the current recommendations are for daily removal and cleaning. Extended wear is possible because SiHG lenses are highly permeable to oxygen (Table 1). The Dk values of 128 for Biofinity™ lenses is higher than other SiHG lenses and even higher than an equivalent thickness of water which is near 80 Dk units. The high Dk values for the Biofinity™ lenses are due to silicone-rich regions which are highly permeable to oxygen. The water content is 48% and the total silicone volume is thought to be about 30%. Even with this high silicone content, the Biofinity™ lens remain highly permeable to water and ions in tears. This permeability is due to the presence of a semi-interpenetrating polymer network (IPN) with continuous channels of essentially pure water or tear fluid, from the front to back surfaces of the lens (FIG. 4). Since the lenses are optically clear, the water channels must be smaller than visible wavelengths light and thought to be about 50 nm in diameter. The IPN structure implies the presence of non-polar to polar interface regions and low polarity regions, which have been demonstrated. The Biofinity™ lenses bound sensing fluorophores which contained hydrophobic side chains to localize the sensing fluorophores at the interface. Similar results were found for MyDay™ lenses which contain a lesser amount of silicone.

The MyDay™ lenses are based on Stenfilcon A™, a 3rd generation SiHG polymer. These lenses were announced in June 2015, and are becoming one of the more frequently prescribed lenses. MyDay™ lenses are prescribed for one-day use and contain a very low silicone content of 4.4%, much less than the Biofinity™ lenses. Even with this low silicone content, the MyDay™ lenses still have high Dk values near 80. The high Dk value is claimed to be the result of continuous silicone channels from the front to the back of the lenses. The IPN network in MyDay™ lenses appears to be the opposite of the Biofinity™ lenses. The MyDay™ lenses contain continuous regions of silicone from the front to the back of the lens (FIG. 4). The non-silicone region of the MyDay™ lenses is probably a non-silicone HG. The MyDay™ lenses were expected to have interface regions for binding of hydrophobic ISF, but this was not certain given the low silicone content. The MyDay™ lenses were expected to be superior to Biofinity™ because of their higher flexibility and higher water content, but recent reports indicate the lenses are nearly identical in terms of patient comfort and corneal health. As a control, binding of hydrophobic ISF-L can be tested with HG Dailies lenses.

Because the incidence of infections is not dependent on the lens material, it is possible that it must be due to the simple physical presence of the lens on the cornea, which affects the electrolyte concentrations in the PLTF and the PolTF. A purpose of certain embodiments of the invention is to develop contact lenses which contain ion-sensitive fluorophores (ISF) which are localized specifically on the PLTF side and/or on the PolTF of the lens. Each side of the lens can be labeled with a different fluorophore, both being sensitive to the same ion. Examples of pairs of fluorophores for pH (W), Na+ and cl− are given herein. This approach provides surface-selected observation based on wavelength or lifetime. Importantly, this approach allows both single-point measurements or imaging of the entire iris or lens area.

The reason for using different fluorophores on the PLTF and PolTF is the unknown rate of water and ion transport across the lenses. A further reason is because it is not known whether blinking results in an immediate mixing of the components in the PLTF or PolTF, or if these volumes remain unmixed after one or more blinking events. The invention allows one to monitor the selected ion on each surface at the same time. The ion concentration in the PolTF can be expected to change due to infection or injury, and the concentrations will remain more constant in the PLTF. The PLTF and PolTF thickness are different, but similar fluorescence intensities would be expected from both sides because the ISF-L are on the lens and not dissolved in the tear film.

### Clinical Relevance

There are an estimated 71,000 cases of microbial keratitis in the United States annually, and contact lens wear has been found to have an adjusted relative risk of 9.31 compared with non-contact lens wearers. As many of these cases progress on to vision loss, extensive efforts have been investigated to prevent and treat this condition. Preventative methods for contact lens wearers involve education on proper lens hygiene, including not sleeping in contact lenses. Unfortunately, even with such precautions, infections still occur. Reliance on symptoms of pain and decreased vision can still result in a delay in care. The ability to measure electrolyte concentrations in the post-lens tear film has the potential to allow for earlier diagnosis and treatment of contact lens-related corneal infections, as infiltration inflammatory markers will change the ionic concentrations of the tear film. This sub-clinical inflammatory response has been shown to the earliest sign of infection, and if this response can be detected by a change in electrolyte concentrations, then the proposed technology could allow for earlier diagnosis of contact lens-related corneal infections. This would result in earlier institution of therapy and better outcomes for patients.

In addition, changes in ion concentration are correlated with many disease conditions. For example, pH of tear fluid correlates with eye infections, chloride correlates with cystic fibrosis, sodium plus chloride correlates with dehydration, and potassium correlates (protection from UV damage). The present invention is contemplated to be useful for testing in any disease or condition that correlates with the presence or amount of an analyte found in tears, including, but not limited to the conditions listed above.

### SUMMARY OF THE INVENTION

Therefore, there is a need in the art for methods for determining the presence and amount of various analytes in tear fluids. Thus, the invention here provides techniques for silicone hydrogel based fluorescent assays that can be used in a microfluidic device, wearable contact lens, DNA or protein array, clinical assay on any solid substrate (preferably with optical transparency), or by immersion assays whereby the target molecules bind to the target-specific silicone hydrogel. A labeled contact lens was developed which can be used in ophthalmology research and the clinic to measure the concentrations of analytes, including ion concentrations, in the tear films which are above and below a contact lens (the top pre-lens tear film (PLTF) and the low post-lens tear film (PolTF).

In a first set of embodiments, a probe composition includes a hydrophobic portion, a hydrophilic portion, an analyte-binding portion and a fluorophore portion. The analyte-binding portion is configured to bind to an analyte in an aqueous solution. The fluorophore portion is configured to change an optical property of fluorescent light emitted in response to incident excitation light when the probe composition changes between a first state in which the analyte is not bound to the analyte-binding portion and a second state in which the analyte binds to the analyte-binding portion.

In a second set of embodiments, a material includes the probe composition and a silicone hydrogel substrate having a hydrogel network that allows flow of aqueous solution through the solution and a silicone network that occupies interstices of the hydrogel network.

In a third set of embodiments, a system includes the material and a remote monitor subsystem configured to detect the change of the optical property of the fluorescent light emitted in response to the incident excitation light without mechanically contacting the material.

In a fourth set of embodiments, a method includes obtaining a silicone hydrogel substrate and contacting the substrate with an aqueous solution that comprises the probe composition as recited above to form a probe-substrate material. The method also includes contacting the probe-substrate material with an aqueous sample solution. Further, the method includes illuminating, using a light source, the probe-substrate material in contact with the sample solution. Yet even further, the method includes measuring a value of a property of the fluorescent light emitted by the probe-substrate material in contact with the sample solution in response to the illuminating. Even further still, the method includes determining a value of a concentration of the analyte in the aqueous sample solution based on the value of the property of the fluorescent light.

In a fifth set of embodiments, a non-transitory computer-readable medium is configured to cause a system to perform one or more steps of the above method.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

In particular embodiments, the present invention relates to a probe composition comprising: a hydrophobic portion; a hydrophilic portion; an analyte-binding portion configured to bind to an analyte in an aqueous solution; and a fluorophore portion configured to change an optical property of fluorescent light emitted in response to incident excitation light when the composition changes between a first state in which the analyte is not bound to the analyte-binding portion and a second state in which the analyte binds to the analyte-binding portion. In some embodiments, the optical property of the emitted fluorescent light is selected from a group consisting of intensity, ratio of intensity among a plurality of frequencies, lifetime of emission, and phase difference from the incident excitation light.

According to some embodiments, the probe composition further comprises a spacer portion configured to place the analyte-binding portion in an aqueous solution when the hydrophobic portion is attracted to an interface with a hydrophobic structure and to place the fluorophore within a certain distance of the analyte-binding portion such that the fluorophore portion is affected by binding of the analyte to the analyte-binding portion.

In some additional embodiments, the fluorophore portion includes an electron donor sub-portion and a separate electron acceptor sub-portion. In addition, in some embodiments, the fluorophore portion includes an electron donor sub-portion and a separate acceptor sub-portion, both sub-portions involved in Forster resonance energy transfer (FRET).

Embodiments of the invention also include a probe composition which is a modular composition wherein the donor sub-portion and the acceptor sub-portion are separate moieties connected by an aliphatic linker, the linker including a diboronic acid.

In some embodiments, the probe composition has the structural formula;

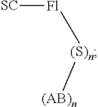

wherein Fl is the fluorophore portion; AB is the analyte binding portion; SC is the hydrophobic portion, the hydrophobic portion comprising a C₈-C1₈ alkyl group; S is a group that provides sufficient spacing between AB and Fl such that when AB binds the analyte, the fluorophore portion changes an optical property of fluorescent light emitted in response to incident excitation light when the probe composition changes between a first state in which the analyte is not bound to the analyte-binding portion and a second state in which the analyte binds to the analyte-binding portion; wherein n represents an integer from 1 to 20; and and wherein the fluorophore portion changes an optical property of fluorescent light emitted in response to incident excitation light when the composition changes between a first state in which the analyte is not bound to the analyte-binding portion and a second state in which the analyte binds to the analyte-binding portion via a photophysical mechanism selected from the group: quenching, photo-induced electron transfer (PET), and intramolecular charge transfer (ICT).

In further embodiments, SC further comprises separating units of polyethelene glycol, hydroxyl groups, or arginine peptide.

The probe composition, in some embodiments comprises an analyte binding portion comprising boronic acid or a diboronic acid. The analyte to be bound by the analyte binding portion, in some embodiments, is selected from a group comprising: glucose, cations of Group I and Group II metals, and anions of Group VIIA.

The invention also includes, in some embodiments, a material comprising: a silicone hydrogel substrate having a hydrogel network that allows flow of aqueous solution through the hydrogel network, wherein a silicone network occupies interstices of the hydrogel network; and the probe composition of claim 1, wherein the hydrophobic portion of the probe composition is attracted to an interface between the hydrogel network and the silicone network.

In these embodiments, the material can optionally further comprise a treated surface of the material, wherein the treated surface has stronger hydrophobic attraction than an untreated surface of the material whereby the concentration of the probe composition is greater on the treated surface of the material than on an untreated surface of the material or internal to the material. The material optionally is incorporated into a contact lens.

Some embodiments of the invention include a material wherein the probe composition is a modular composition wherein the donor sub-portion and the acceptor sub-portion are separate species connected by an aliphatic linker, the aliphatic linker including a diboronic acid; and wherein the separate donor sub-portion and the separate acceptor sub-portion are a pair of separate species selected from a group of pairs consisting of: Quinolinium C-18 paired with 7-nitrobenz-2-oxa-1,3-diazol-4-yl (NBD) with a C18 side chain (NBD C-18); Naphthalene paired with Dansyl; Dansyl paired with fluorescein-5-isothiocyanate (FITC); Dansyl paired with octadecylrhodamine (ODR); 1-N6-ethenoadenosine (s-A) paired with NBD; IAF paired with tetramethylrhodamin (TMR); Pyrene paired with coumarin; FITC paired with TMR; 5-(2-((iodocetyl)amino)ethyl)amino)naphthalene-1-sulfonic acid (IAEDANS) paired with FITC; IAEDANS paired with 5-iodoacetamidofluorescein (IAF); IAF paired with an enzyme immunoassay (EIA); carboxylfluorescein, succinimidyl ester (CF) paired with Texas Red (TR); 4,4-difluoro-4-bora-3a,4a-diaza-s-indacene (Bodipy) paired with Bodipy; B-phycoerythrin (BPE) paired with a cyanine dye (Cy); Terbium paired with Rhodamine; Europium paired with Cy; and Europium paired with allophycocyanin (APC).

In certain embodiments, the acceptor sub-portion further comprises one or a plurality of halogen groups.

In certain embodiments, the probe composition has a longitudinal axis length between 2-8 nm when the analyte is not bound to the analyte binding portion.

In some embodiments, the probe composition has the structural formula:

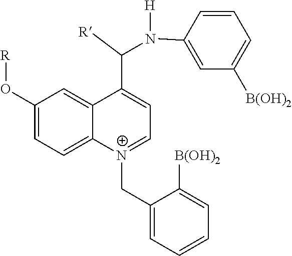

wherein R is one of the species selected from the group consisting:

a C₈-C₁₈ alkyl group;

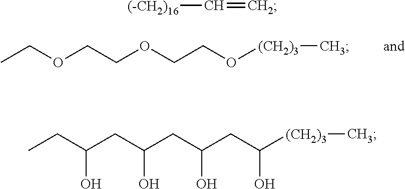

and
wherein R′ is hydrogen or a ketone functional group.

In some embodiments, the probe composition has the structural formula:

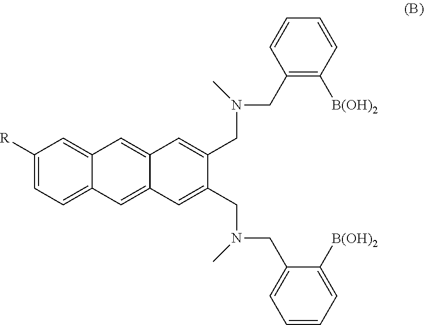

wherein R is one of the species selected from the group comprising:
a C₈-C₁₈ alkyl group;

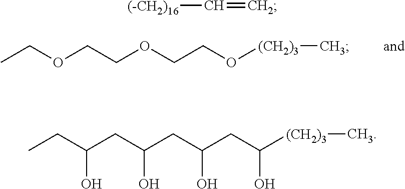

The probe composition, in some embodiments, has the structural formula:

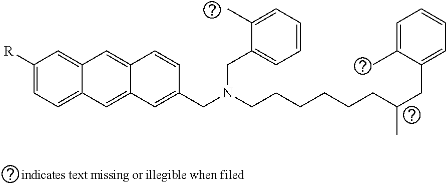

wherein R is one of the species selected from the group comprising:
a C₈-C₁₈ alkyl group;

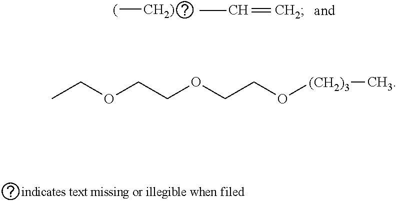

The probe composition, in some embodiments, has the structural formula:

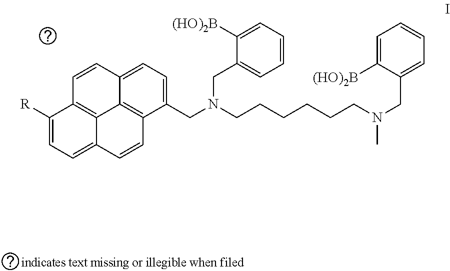

wherein R is one of the species selected from the group comprising:
a C₈-C₁₈ alkyl group

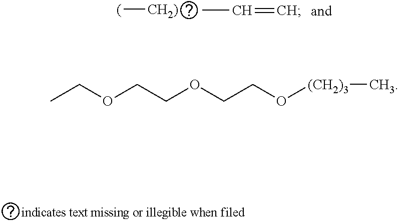

The probe composition, in some embodiments, has the structural formula:

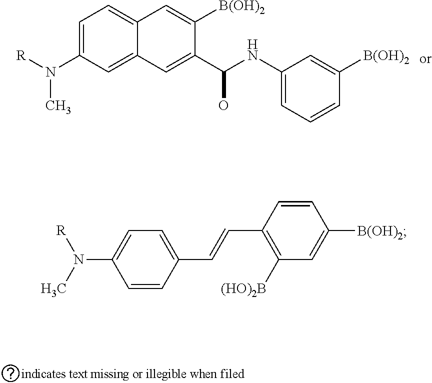

wherein R is one of the species selected from the group consisting of:
a C₈-C₁₈ alkyl group;

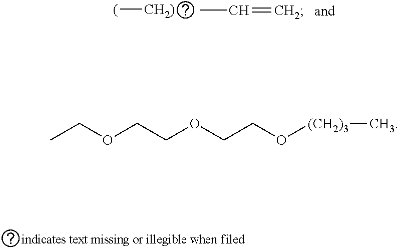

The probe composition, in some embodiments, has the structural formula:

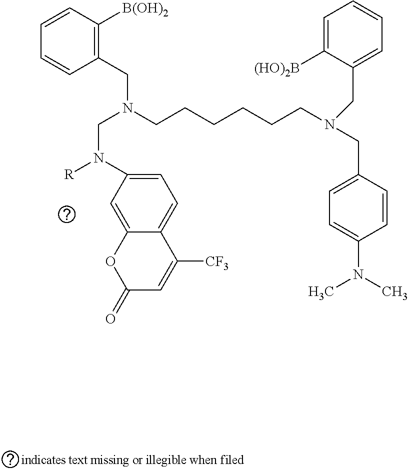

wherein R is one of the species selected from the group comprising:
a C₁-C₈ alkyl group.

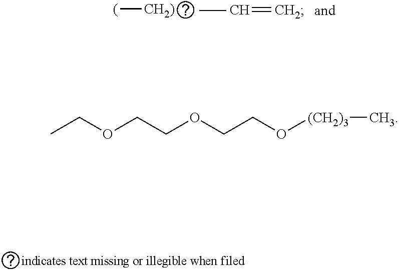

The probe composition, in some embodiments, has the structural formula: and

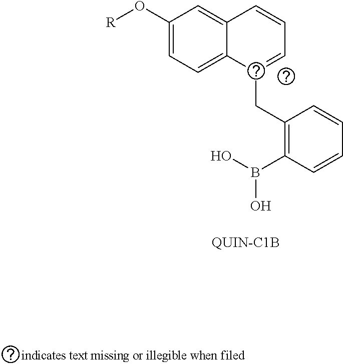

wherein R is one of the species selected from the group comprising:
one or a plurality of C₈-C₁₈alkyl groups

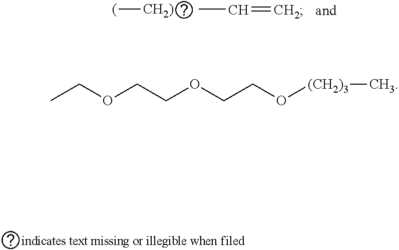

The material, in certain embodiments, is configured to detect pH levels wherein the probe composition is a quinolinium based probe composition having a hydrophobic side chain comprising between 8-18 carbon atoms.

The material, in certain embodiments, is configured to detect ion concentrations of at least one Group I, Group II, or Group VIIA element.

The material, in certain embodiments, includes a probe composition is configured to detect cations of Group I metals and wherein the probe composition has the structural formula selected from the group:

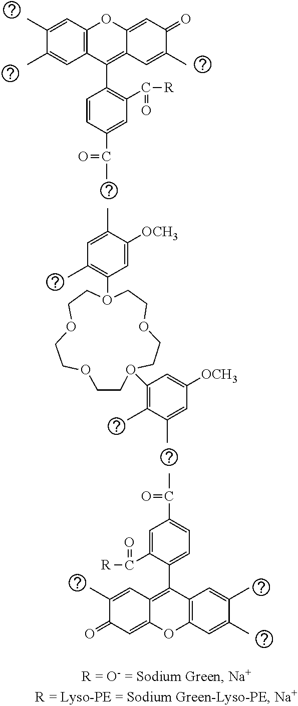

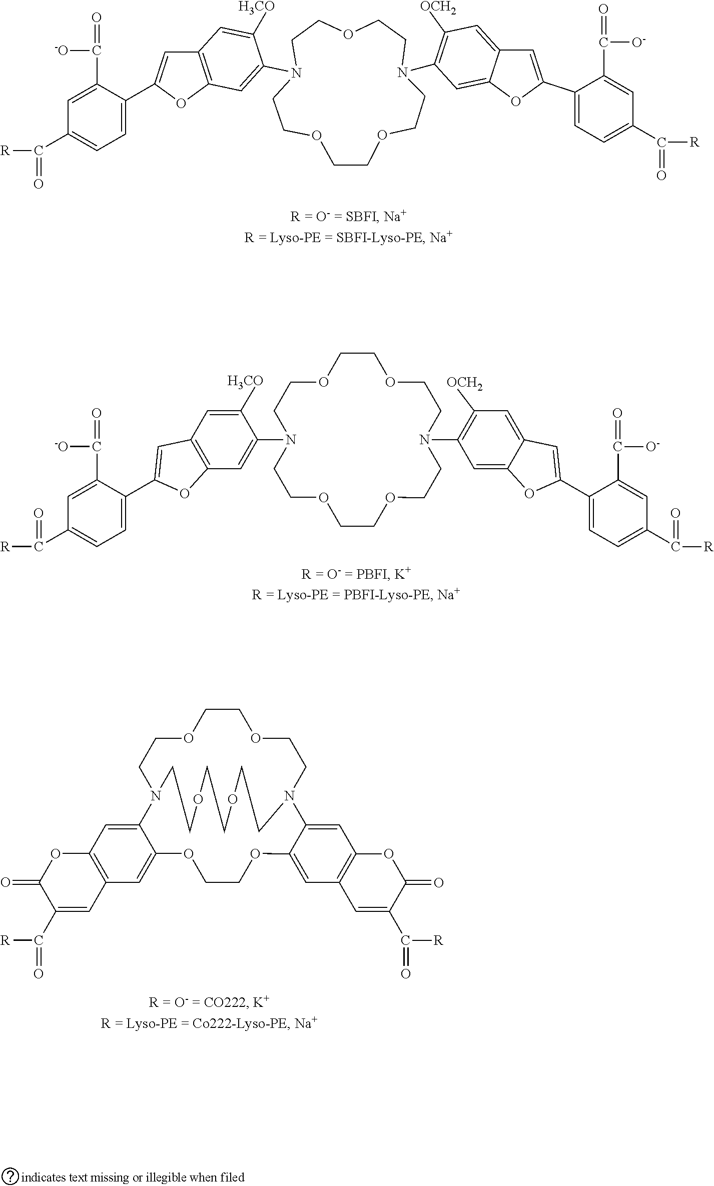

In some embodiments, the probe composition is configured to detect cations of Group II metals, and wherein the probe composition has the structural formula selected from the Group:

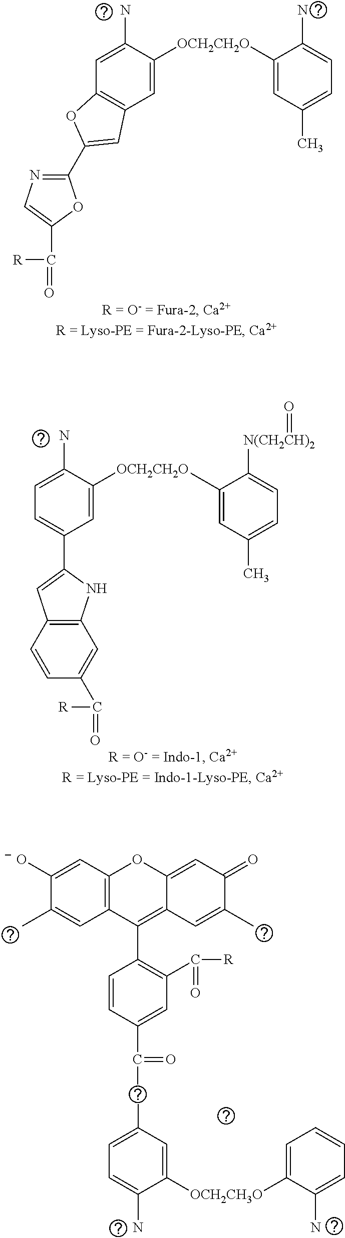

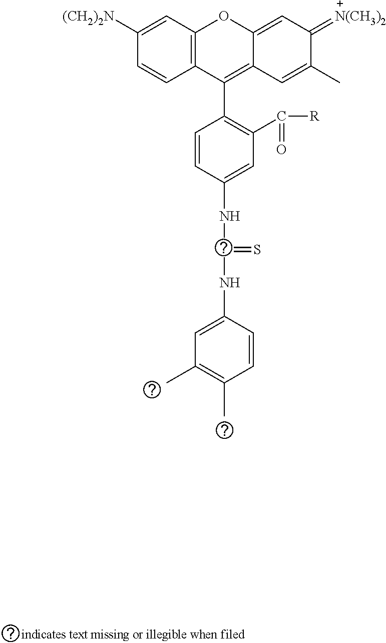

In some embodiments, the probe composition is configured to detect an anion of a Group VIIA element and wherein the probe composition has the structural formula selected from the group:

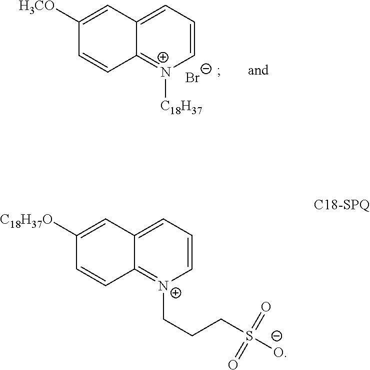

The invention also comprises, in some embodiments, a system comprising: the material of claim 11; and a remote monitor subsystem configured to detect the change of the optical property of the fluorescent light emitted in response to the incident excitation light without mechanically contacting the material. This system can include embodiments wherein the material is fixed to a microfluidic device and/or wherein the material is incorporated into a contact lens.

In additional embodiments, the monitor subsystem further comprises: an incident light source; a light detector; and a processing system, the processing system further comprising at least one processor; and at least one memory including one or more sequences of instructions, the at least one memory and the one or more sequences of instructions configured to, with the at least one processor, cause the system to perform at least the following, operate the incident light source to illuminate the material, operate the light detector to obtain data that indicates the property of the emitted fluorescent light, and determine a concentration of the analyte based on the data that indicates the property of the emitted fluorescent light.

The invention also includes, in some embodiments, a system wherein: the system further comprising an analyte response device; and the at least one memory and the one or more sequences of instructions are further configured to, with the at least one processor, cause the system to operate the analyte response device based on the concentration of the analyte.

The invention also includes, in certain embodiments, a method comprising:

obtaining a silicone hydrogel substrate; contacting the substrate with an aqueous solution that comprises the probe composition as recited in claim 1, wherein the composition is a probe, to form a probe-substrate material; contacting probe-substrate material with an aqueous sample solution; illuminating, using a light source, the probe-substrate material in contact with the sample solution; measuring a value of a property of the fluorescent light emitted by the material in contact with the sample solution in response to the illuminating; and
determining a value of a concentration of the analyte in the aqueous sample solution based on the value of the property.

In some embodiments, the step of determining the concentration of the analyte is performed automatically on a processor.

In some embodiments, the method further comprises operating an analyte response device based on the value of the concentration of the analyte in the aqueous sample solution.

Other embodiments of the invention, included a non-transitory computer-readable medium carrying one or more sequences of instructions, wherein execution of the one or more sequences of instructions by one or more processors causes the one or more processors to perform the steps of: operating an incident light source to illuminate the material, operating a light detector to obtain data that indicates the property of fluorescent light emitted in response to operating the incident light source, and determining a concentration of the analyte based on the data that indicates the property of the emitted fluorescent light.

In some embodiments, execution of the one or more sequences of instructions by one or more processors further causes the one or more processors to perform the step of operating an analyte response device based on the concentration of the analyte.

### BRIEF SUMMARY OF THE DRAWINGS

Certain embodiments are illustrated by way of example, and not by way of limitation, in the figures of the accompanying drawings in which like reference numerals refer to similar elements and in which:

FIG. 1A and FIG. 1B are drawings showing the structure of the cornea. FIG. 1A shows the entire cornea. FIG. 1B shows the tear film, expanded. FIG. 1C shows the position of a contact lens floating in the central aqueous region of the tear film.

FIG. 2 is a schematic drawing showing the PoLTF and PLTF on the eye surface.

FIG. 3A is an expanded view of the cornea, contact lens and tear films. Green regions are surface-localized fluorophores near the PolTF, and red indicates fluorophores near the PLTF. FIG. 3B shows the cornea wound ion currents.

FIG. 4A is a drawing showing the semi-interpenetrating polymer networks for SiHG contact lens with water channels. Light blue indicates silicone regions and light pink indicates the water or tear fluid channels. The green dots indicate the locations of the ionic species from the tears in the aqueous channels. FIG. 4B, SiHG lens with silicon channels through a water or HG phase. FIG. 4C shows an HG contact lens with no silicone.

FIG. 5 shows the chemical structure of a monomer present in a SiHG contact lens, according to an embodiment.

FIG. 6A shows a modern time-resolved spectrofluorometer. FIG. 6B shows an electronic board for a complete time-resolved fluorometer. FIG. 6C provides examples of fluorescence measurements by intensity, intensity ratio, time-domain, frequency-domain (FD) methods.

FIG. 7A and FIG. 7B provide intensity decay results for an anthracene-like probe, ANDBA, in buffer. FIG. 7A shows data collected for 100, 10 or 1 seconds. Incident power at 375 nm is less than 5 mW/mm². FIG. 7B shows data collected with 10, 1 or 0.1% of the maximum intensity of 0.5 mW.

FIG. 8A, FIG. 8B, and FIG. 8C show instrumentation suitable for use with the invention. FIG. 8A is an image of a cell phone camera. FIG. 8B is an image of a CMOS chip with all electronics. FIG. 8C is an image of a CMOS chip for imaging without electronics.

FIG. 9A and FIG. 9B are Stern-Volmer plots for chloride quenching of SPQ-C18 in Biofinity™ and MyDay™ contact lenses.

FIG. 10A and FIG. 10B are block diagrams that illustrate an example probe-substrate material, according to an embodiment. FIG. 10C is a block diagram that illustrates an example probe composition, according to an embodiment. FIG. 10D is a block diagram that illustrates example fluorescent light properties that can be measured, according to various embodiments.

FIG. 11 is a flow chart that illustrates an example method for measuring the concentration of an analyte based on a probe-substrate material, as depicted in FIG. 10A, according to an embodiment.

FIG. 12A and FIG. 12B show 1,8-ANS in water-methanol mixtures, acetonitrile and MyDay™ lens. ANS in solvents are with the same concentration. Excitation wavelength is 375 nm. Insert shows labeled lens on a UV handlamp.

FIG. 13A and FIG. 13B are block diagrams that illustrate example systems that detect concentration of an analyte in fluid from a subject, according to some embodiments.

FIG. 14 is a block diagram that illustrates a computer system upon which an embodiment of the invention may be implemented.

FIG. 15 illustrates a chip set upon which an embodiment of the invention may be implemented.

FIG. 16 is a diagram of exemplary components of a mobile terminal (e.g., cell phone handset) for communications, which is capable of operating in the system, according to one embodiment.

FIG. 17A and FIG. 17B are images that illustrate a contact lens that can be crafted using materials containing probe compositions to detect one or more various analytes, including glucose, according to various embodiments.

FIG. 18A is graph that illustrates an example carboxy SNARF-6 absorption spectra and effect of pH on the spectra, according to an embodiment. FIG. 18B and FIG. 18C are graphs that illustrate example time dependent decays recovered from full frequency-domain measurements and analysis at a pH where the total steady state intensities are close to equal, according to an embodiment.

FIG. 19A is a chemical diagram that illustrates an example synthesis scheme for a probe composition for use to measure and detect pH in a contact lens, according to an embodiment. FIG. 19B is a set of images that illustrates how an example Quin C-18 probe fluoresces at various pH levels when exposed to UV light, according to an embodiment.

FIG. 20 is a graph that illustrates an example excitation spectra (left) and emission spectra (right) of 6-OH—N-C18H37-QBr in Biofinity™ contact lens in water, according to an embodiment.

FIG. 21 is a graph that illustrates example intensity decay of 6OH—N-C18H37-QBr in a Biofinity™ contact lens in water, according to an embodiment.

FIG. 22 is a chemical diagram that illustrates an example synthesis scheme for 6-OH—N-Allyl-QBr, according to an embodiment.

FIG. 23 is a chemical diagram that illustrates example hydrophobic side chains which may be incorporated into a certain pH-detecting probe compositions disclosed, such that the hydrophobic portion binds the probe composition at an interface in SiHG lens, as well as an example pH probe having a hydrophobic side chain, according to an embodiment.

FIG. 24 is a set of chemical diagrams that illustrate example chemical structures of an Na⁺ probe and ultraviolet (UV) analogues (i.e. SBFI), for use with UV light, according to various embodiments.

FIG. 25A is a graph that illustrates example Na⁺ and K⁺ dependent emission spectra of SBFI, according to various embodiments. FIG. 25B is a graph that illustrates example Na⁺ and K⁺ dependent emission spectra of PBFI, according to an embodiment.

FIG. 26A and FIG. 26B show sodium-dependent emission spectra (FIG. 26A) and intensity decays (FIG. 26B) of SG-PL in MyDay™ contact lens. Λₑₓ=495 nm and λₑₘ=545 nm. IRF is the instrument response function.

FIG. 27A and FIG. 27B show binding curves of sodium (log scale) to SG-PL for Biofinity™ and MyDay™ lenses as measured by intensities (FIG. 27A) or amplitude-weighted lifetimes (FIG. 27B). Numerical values indicate the mid-points of the sodium response. The inserts show reversibility between 0.0 and 340 mM.

FIG. 28A is a set of photographs of SG-PL in a Biofinity™ contact lens with diffuce 473 nm illumination for 0 and 140 mM NaCl. FIG. 28B is a set of confocal lifetime images of S2C16 in Biofinity™ contact lenses for 0 and 140 mM NaCl.

FIG. 29 is a set of chemical diagrams that illustrate example structures of calcium and magnesium ion-detecting probe compositions and proposed lyso-PE derivatives for binding the probe composition into a contact lens, according to various embodiments.

FIG. 30A and FIG. 30B are graphs that illustrate example absorption and emission response, respectively, to magnesium as well as a structure for a magnesium-detecting probe composition, according to an embodiment.

FIG. 31 is a set of diagrams that illustrate various example mechanisms by which changes in geometry of a boronic acid moiety can affect the spectra, intensity, or lifetimes of nearby fluorophores.

FIG. 32 is a chemical diagram that illustrates example probe composition structures for detecting chloride ions, according to an embodiment.

FIG. 33A is a graph that illustrates an example emission spectrum for SPQ-18 in an Stenfilcon (Aspire™) contact lens, according to an embodiment. FIG. 33B is a graph that illustrates an example time-dependent decay of SPQ-18 in the presence of the chloride ion, according to an embodiment.

FIG. 34 is a graph that illustrates an example comparison of lifetime Stern-Volmer traces for SPQ-C3 in water and SPQ-C18 in a Stenfilcon (Aspire™) contact lens, according to an embodiment.

FIG. 35 is a chemical diagram that illustrates an example generic diboronic acid molecule in the sugar-bound and sugar-unbound conformations, according to an embodiment.

FIG. 36 is a chemical diagram that illustrates a more detailed example reaction scheme showing the binding of boronic acids to sugars, showing different forms of the various boronic acid moieties, according to an embodiment.

FIG. 37 is a graph that illustrates example intensity decays in THF and Biofinity™ contact lenses, according to an embodiment.

FIG. 38 is a graph that illustrates example anisotropy decays in THF and Biofinity™ contact lenses, according to an embodiment.

FIG. 39 is chemical diagram that illustrates an example reaction scheme for the preparation of probe composition Quin C-18, according to an embodiment.

FIG. 40 is a graph with image inset that illustrates an example of persistence of fluorescent probe in SiHG contact lens after washing by showing the emission spectra of Quin-C-18 after repeated washing, and a photograph of the lens in room light and with UV incident light, according to an embodiment.

FIG. 41A is graph that illustrates an example glucose-dependent emission spectra of Quin-C18 within a Biofinity™ SiHG contact lens, according to an embodiment. FIG. 41B is a graph that illustrates example normalized intensities in a Dailies (HG) lens and in three SiHG, Biofinity™, Stenfilcon A (Aspire™ 1 day) and Optix-Aqua™ lenses, according to an embodiment.

FIG. 42 is a chemical diagram that illustrates example diboronic acid Glu-SFs structures using a quinolinium nucleus for binding at an interface in SiHG contact lens, according to an embodiment.

FIG. 43 is a chemical diagram that illustrates example diboronic acid PET Glu-SFs for binding at interfaces in SiHG lenses, in which the lower two structures are to displace the diboronic acid (DiBA) more into the water phase, according to an embodiment.

FIG. 44 is a set of chemical diagrams that illustrate diboronic acid ICT Glu-SFs for binding at interfaces in SiHG lenses, according to an embodiment.

FIG. 45A and FIG. 45B are chemical diagrams that illustrate Glu-SF structures using a diboronic acid on a C6 linker with FRET mechanism, according to an embodiment and a Glu-SF structures using a diboronic acid on a C6 linker with a collisional quenching mechanism, according to an embodiment.

FIG. 46 is a block diagram that illustrates an example measurement system, according to an experimental embodiment, for measuring fluorescence from surfaces of contact lenses on rabbits.

FIG. 47 is a graph showing LSCFM of two layers of FL-PL in solution separated with glass cover slips of 123 μm thickness. Objective 20×, NA 0.40, pinhole 25 μm. The half-width is 20-22 μm which includes the thickness of FL-PL layers estimated as about 4-5 μm.

FIG. 48A is a schematic drawing showing the experiment in pictorial form. FIG. 48B and FIG. 48C show the z-axis intensities of Biofinity™ and MyDay™ lenses, respectively. The lenses are labeled with fluorescein-polylysine and fluorescein C16. The dotted line shows z-resolution. Dashed lines show approximate inner and outer surfaces of lenses.

FIG. 49A and FIG. 49B show pH-dependent absorption and emission spectra of BCECF (FIG. 49A) and SNARF-1 (FIG. 49B).

FIG. 50A shows carboxy-SNARF-6 time-dependent decays recovered from full frequency-domain measurements and analysis of the single and multi-experimental analysis with 543 nm excitation. FIG. 50B shows the pH-dependent phase lifetime of carboxy SNARF 6. Phase lifetimes are calculated from phase angles at modulation frequency of 135 MHz.

FIG. 51A shows pH-dependent equilibrium between the neutral and anionic forms of 6HQ-C18. FIG. 51B is a set of photographs of 6HQ-C18-labeled Biofinity™ CL in pH 4.0 (left) and 10 (right) under room light with a UV handlamp.

FIG. 52A and FIG. 52B show excitation and emission spectra, respectively, of 6HQ-C18 in Comfilcon A Biofinity™ lenses. FIG. 52C shows the pH-dependent excitation wavelength ratio for 6HQ-C3 in buffer and 6HQ-C18 within Comfllcon A-SiHG lenses. Emission was monitored at 580 nm and Aex=350 nm.

FIG. 53A and FIG. 53B show the structure (FIG. 53A) and fluorescence emission (FIG. 53B) of sodium-sensitive fluorophores for the PLTF and PoLTF.

FIG. 54 shows the chemical synthesis and structures of the Sodium Green linked to poly-lysine (SG-PL) or 1-amino hexadecane (SG-2C16) for use in contact lenses.

FIG. 55A and FIG. 55B show emission spectra and intensity decays, respectively, of SG-2C16 bound to a Biofinity™ contact lenses, with increasing concentrations of sodium.

FIG. 56A, FIG. 56B, and FIG. 56C provide the chemical structures, absorption and emission, respectively, of potassium-sensitive fluorescent probes.

FIG. 57A and FIG. 57B show the chemical structures and emission, respectively, of Calcium Green and Calcium Crimson, AM.

FIG. 58A, FIG. 58B, and FIG. 58C, respectively, show the chemical structures, absorption, and emission, of Mag-quin-2 and Mg-Green.

FIG. 59A and FIG. 59B present the structures and spectral properties of chloride-sensitive fluorophores. The absorption and emission spectra of OD-MQB are nearly identical to SPQ shown in the figure.

FIG. 60A and FIG. 60B are photographs of a lens labeled at one spot with SG-PL immediately after spotting (FIG. 60A) and after 4 days in buffer (FIG. 60B). Intensity line tracings are shown along the dashed red lines on the photographs in FIG. 60C.

FIG. 61A and FIG. 61B are fluorescence photographs taken immediately after probe binding (FIG. 61A) and after incubation in buffer for 4 days (FIG. 61B). FIG. 61C shows an intensity line tracing.

FIG. 62A and FIG. 62B show examples of a contact lens for measurements of a single ion (FIG. 62A) or multiple ions (FIG. 62B) concentrations in tears.

FIG. 63A shows a rabbit in a restrainer, used for measurements. FIG. 63B and FIG. 63C show emission spectra and intensity decays, respectively, of SG-PL in a Biofininty™ lens on a rabbit eye, with and without 150 mM Na⁺.

### DETAILED DESCRIPTION

### 1. Overview

A composition, material, method, apparatus and system are described for a silicone hydrogel based assay and contact lens. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Embodiments of the invention described herein include new techniques for fluorescence assays that take advantage of the structure of silicone hydrogels to affix assay specific fluorescent probes (also referred to as “probe compositions”) to the silicone interstices between hydrogel nanochannels that allow the flow of aqueous sample solutions with analyte past the fixed probes. The probes require at least a hydrophobic portion to be attracted to the silicone interstices, a hydrophilic portion to maintain contact with the aqueous sample solution, an analyte-binding portion to capture an analyte from the sample solution, and a fluorophore portion that will change at least one measureable property of its fluorescent emission when an analyte binds to the analyte-binding portion. In some embodiments, the fluorophore portion is made up of several separate sub-portions. In some embodiments one or more spacer portions are included in the probe to ensure that the analyte-binding portion and fluorophore portion have the proper spatial relationships to interact and demonstrate the desired functionality. Because the probe need not be a single molecule, the probe is also called a probe composition herein.

Some embodiments of the invention are described below in the context of contact lenses made of a material that includes a silicone hydrogel substrate and fluorescent probes attracted to a water-silicone interface of the substrate, for which fluorescence is measured remotely. However, the invention is not limited to this context. In other embodiments the material is part of a microfluidic device or other medical device that comes in contact with one or more fluids of a subject, or the fluorescent measurement is performed by a subsystem embedded in the substrate or mechanically in contact with the substrate. In certain embodiments, the invention is used with surface-based testing such as DNA and protein arrays, or clinical assays on substrates or by immersion of silicone hydrogel into the sample followed by such measurements.

These techniques enable one to determine the presence and concentration of analytes in tear fluid of a subject's eye using a low-cost commercial contact lens as a substrate and readily available imaging technology as a remote monitoring sub-system. For at least these reasons, the new techniques are superior to previous approaches. In addition, the inventive techniques can be used in a microfluidic device deployed inside or outside the eye of a subject to measure other fluids in addition to, or instead of, tear fluid, of the same or different subjects.

### 1. DEFINITIONS

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” means plus or minus 20 percent of the recited value, so that, for example, “about 0.125” means 0.125±0.025, and “about 1.0” means 1.0±0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the term “subject” refers to an animate or inanimate object, which can include a geological formation, a machine, or a living organism including a plant or an animal, including a human. “A subject in need” is a subject wherein an analysis of fluid for specific analytes in a small volume is desirable.

As used herein, the term “concentration” refers to a numerical value for the amount (weight or volume) of an analyte in a volume of a fluid, including a binary determination whether the amount is above some measurable threshold, i.e., is present, in a sample.

### 2. SUMMARY OF RESULTS

Results from this laboratory demonstrated the use of fluorescently-labeled contact lens to measure electrolytes and sugars in simulated tear fluids. When a contact lens is inserted into the eye, it localizes in the central aqueous layer which contains the ions and proteins.

Fluorescence sensing can be accomplished by several methods. Steady state intensity measurements can be difficult to use for many reasons, including eye movement, blinking and changing efficiency of collecting the emission. One method to avoid these difficulties is to use wavelength-ratiometric (WR) measurements. WR measurements are only useful if the fluorophore displays a different absorption or emission spectrum with or without the bound ion. Such change occurs for some ISF such as the pH probe described here, but more commonly there are only changes in intensity rather than useful spectral shifts. This problem can be overcome by addition of a second fluorophore which is not sensitive to the measured ions, but this approach requires an additional fluorophore. For measurement of two ions in the PLTF and PoLTF, two reference fluorophores are required, and the emission from four fluorophores will be difficult to resolve. When multiple ionic species need to be measured, the WR method does not appear to be practical. The problems of simple intensity-based measurements can be avoided by lifetime-based sensing.

According to several embodiments provided herein, the disclosure provides (a) a description of the properties silicon hydrogels (SiHG) contact lens which makes them useful for fluorescence sensing, (b) methods to bind ion-sensitive fluorophores (ISF) to the surface of contact lenses, (c) lifetime-based and wavelength-ratiometric measurements of fluorescence which are independent of total intensity, allowing measurements on an eye which is moving, and (d) examples of pairs of ion-sensitive fluorophores (ISF) which have different spectral properties and responsive to the same ionic species, but are localized on the front and back surfaces of the contact lens. These concentrations can differ because the corneal cells are active in ionic transport and transport selective ions.

### 3. EMBODIMENTS OF THE INVENTION

New techniques for fluorescent assays are described herein that take advantage of the structure of silicone hydrogels to affix assay-specific fluorescent probes (also referred to as “probe compositions”) to the silicone interstices between hydrogel nanochannels in the body of a contact lens that allow the flow of aqueous sample solutions with analyte past the fixed probes. In certain embodiments, the probes require at least four features: (1) a hydrophobic portion to be attracted to the silicone interstices, (2) a hydrophilic portion to maintain contact with the aqueous sample solution, (3) an analyte-binding portion to capture an analyte from the sample solution, and (4) a fluorophore portion that changes at least one measurable property of its fluorescent emissions when an analyte binds to the analyte-binding portion of the probe composition. In some embodiments, the fluorophore portion is made up of several separate sub-portions. In some embodiments, one or more spacer portions are included in the probe composition to ensure that the analyte-binding portion and fluorophore portion have the proper spatial relationships to interact and demonstrate the desired functionality. Because the probe need not be a single molecule, the probe is also called a probe composition herein.

These techniques enable the determination of the concentration of analytes in tear fluid of a subject's eye using a low-cost commercial contact lens as an assay substrate and readily available imaging technology as a remote monitoring sub-system. For at least these reasons, the techniques described here are superior to previous approaches. In addition, the new techniques can be used in a microfluidic device deployed inside or outside the eye of a subject to measure other fluids in addition to, or instead of, tear fluid, of the same or different subjects. As used herein, a subject is an animate or inanimate object including a geological formation, a machine, or a living organism including a plant or an animal, the later including a human.

Specific analytes of interest that have been the target of previous approaches to determine the concentration in tear fluid include: glucose for a subject with diabetes; electrolyte imbalance for Dry Eye Syndrome (DRY, also known as Dry Eye Disease, DED); and defensins and other biomarkers for infection. (Defensins are small peptides with 29 to 42 amino acids that are constrained into folded forms by six conserved cysteine residues.)

One attempt to avoid tear collection and thus minimize eye irritation is electrochemical detection of tear glucose in situ using a specially fabricated contact lens called the GLUCOLENS™. This product is being developed by the Google X project of Alphabet, Inc, (Mountain View, Calif.). The GLUCOLENS™ includes electronic components and glucose sensors to allow continuous glucose measurements, and is powered by an induction coil also embedded in the lens, positioned to be placed to encircle a pupil of a subject wearing the lens. The operating principle of glucose self-testing kits and the GLUCOLENS™ is based on the same principle as in the first glucose electrodes and present glucometers; i.e. glucose oxidase and electrochemical H₂O₂ detection. The need for complex embedded electronics increases the cost of the approach and may prevent this device from becoming a daily use product. A daily use product is preferred for use, especially in developed countries, for reasons of safety and patient choice. The suitability of the approach for analytes and biomarkers has not been demonstrated and is likely to be hindered by the cost of the embedded electronics.

Attempts to use fluorophores that respond to analyte concentrations within commercial contact lenses have been hindered by the inability to fix the fluorescent probes in the contact lens and the changes in analyte dependent fluorescence when in the contact lens chemical and physical environment. Furthermore, typical probes have failed to respond in HEMA-type contact lenses.

For example, most publications on glucose sensing focus on boronic acid probes (as an alternative to glucose oxidase used in the current electrochemical glucometers), where boronic acids bind reversibly to glucose. This binding alters the fluorescence spectral features obtained from several fluorophores, which can be correlated to the glucose levels in a sample of interest. With one exception, none of the previous boronic acid (BA) fluorescent probes developed for glucose sensing in buffer conditions were able to respond to the glucose levels within a contact lens. The one exception is a probe based on quinolinium derivatives which gave a small response to glucose in contact lens using a hydrogel polymer (Nelfilcon A™) and washed out of the contact lens. Many other boronic acid probes provided no response to glucose in the hydrogel lenses. These are not considered useful for daily use as contemplated for embodiments of this invention.

Designing a suitable glucose sensitive fluorophore (Glu-SF), for inclusion in a glucose sensitive contact lens (Glu-CL) and for remote optical measurements of tear glucose, has met several barriers. Known Glu-SFs required ultraviolet (UV) or near-UV excitation and practical, portable light sources did not yet exist at the time of those previous attempts. Glu-SFs for longer wavelength excitation were either not available or displayed minimal spectral changes. A barrier to the design of Glu-SFs was the rapidly changing polymer chemistry for contact lenses. A Glu-SF which responds in poly-hydroxyethyl-methacrylate (HEMA) hydrogels (HGs) from the 1990s may not respond in the current lenses based on silicone hydrogels (SiHGs) emerging after 2000.

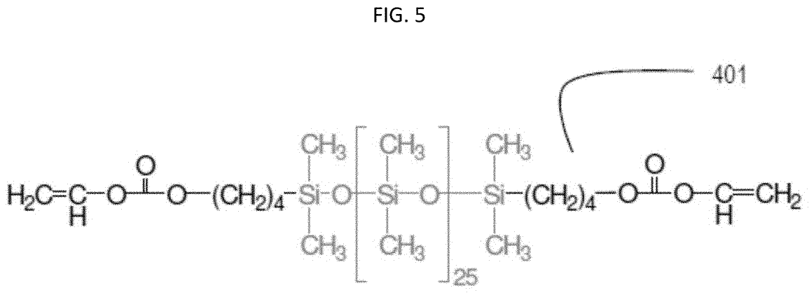

Use of problematic hydrogel contact lenses, which are fragile and have Dk values that could not be increased above that of pure water, was solved when silicone hydrogels (SiHG) were developed. A typical monomer used in these lenses 401 is shown in FIG. 5 and contains a long silicone backbone and carbon-containing reactive groups at the ends for polymerization. Many different monomers and monomers combinations are used. These soft contact lenses contain variable proportions of cross-linkers to control the water content and structural rigidity. The most important feature of silicone hydrogels (SiHGs) is that the Dk values for oxygen transport are dramatically increased compared to non-silicone HGs. The Dk values of SiHG lenses now are about 3-fold larger than a comparable thickness of water.

The first commercial SiHG lens (appearing in 1998) was popular with patients because of comfort and softness, but some patients described problems of eye dryness and inflammation. These adverse effects were discovered to be due to the hydrophobicity of silicone and interference with the tear layer. These problems were solved by making the lens surfaces hydrophilic using chemical or plasma surface oxidation. The resulting lenses found acceptance by a high proportion of the patients. Such lenses are approved for daily or long-term wear. Now, over 70% of new prescriptions for contact lenses are for SiHG lenses (SiHG-CL). Because of mass production, such SiHG-CL have become very inexpensive. As a consequence, in developed countries, one day use-and-disposal has become the preferred mode of use, even with extended wear lenses.

By including fluorophores in the contact lens and using remote electronics to determine concentration of analyte as shown in this specification, the cost of a Glu-CL can be reduced to be comparable with a plain SiHG-CL.

In order to selectively modify the posterior surface of contact lenses for the estimation of analytes in the cornea-contact lens interface film in the PoLTF directly above the epithelium (at the surface of the epithelium) the posterior surface of the contact lens was selectively labeled with analyte-sensitive fluorophores (ASFs). Such fluorophores can be sensitive to ions (Na⁺, H⁺ K⁺ Ca²⁺ Mg²⁺ and Cl⁻) and/or to other analytes of interest. ASFs can be prepared outside and adsorbed to these regions, or as an alternate approach, ASFs can be grafted on surface of CLs directly.

Contact lenses which contain ion-sensitive fluorophores (ISF) only on the posterior surface are suitable for providing analytical chemical information on the region directly above the epithelium. Laser scanning fluorescence confocal microscopy (LSFCF) can be used to confirm the location of the probe composition(s) at the appropriate place on the contact lens. The ability to measure/locate these fluorophores depends on the z-axis (axial) resolution of the instrument. In confocal microscopy, the xy-plane and z-axial resolution are given by dxy=1.22×/2NA and dz=2×n/(NA)2 where λ is the wavelength, n is the refractive index and NA is the numerical aperture of the objective.

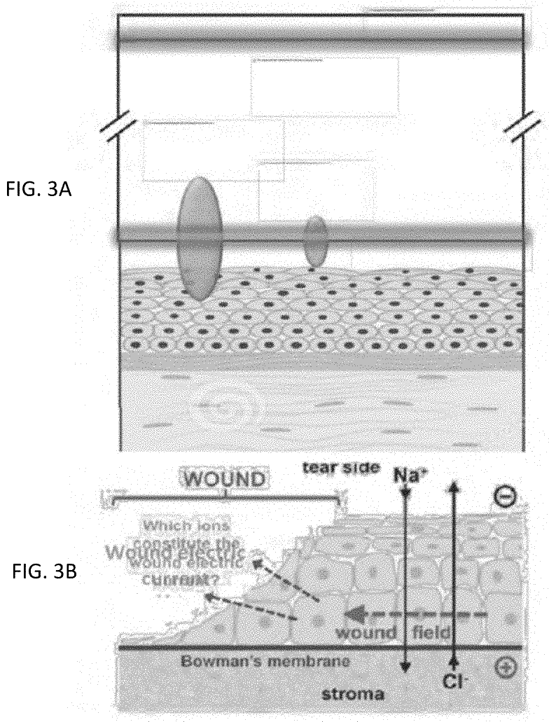

In the preliminary experiments we used a 20×, NA=0.40 objective, and n=1.5, at 550 nm. The diffraction limited resolutions are dxy=839 nm and dz=10.2 μm. The in-plane x-y resolution is more than adequate for imaging x-y planes of the cornea and tear films. The z-resolution with the 20× objective appears to be adequate for separate detection of the PLTF and the PolTF as can be seen from the effective observed volume placed on the contact lens and cornea (see ovals in FIG. 3). When centered on the PoLTF, the volume will extend into the contact lens and into the epithelium. Signal from the inner part of the contact lens would not be expected. Emission from the epithelium can be avoided by using Jong wavelength fluorophores with excitation and emission above 440 nm. If needed, the observed volume can be moved into the lens and away from the epithelium for more selective excitation of the fluorophores on the back side of the lens. The z-resolution can be dramatically increased using a higher NA objective. For example, using the 100×, NA=0.90 air objective the resolution values are dxy=373 nm and dz=2.0 μm. Higher resolution can be obtained using immersion objectives. A 60× NA=1.4 immersion objective has a z-resolution of 0.84 μm and even higher z-resolution has been reported for similar objectives. In addition, one can separately observe emission from the PLTF. When the lenses have two different fluorophores each probe can be observed at a different wavelength without confocal detection.

Specific analytes of interest that have been the target of previous approaches to determine the concentration in tear fluid include: glucose for a subject with diabetes; electrolyte imbalance for Dry Eye Syndrome (DRY, also known as Dry Eye Disease, DED); and defensins and other biomarkers for infection. (Defensins are small peptides with 29 to 42 amino acids that are constrained into folded forms by six conserved cysteine residues.)

Certain of the devices and methods are configured to have high sensitivity and specificity by using fluorescent labeling. In some embodiments, the devices are configured to perform intensity-independent measurements. In addition, the devices optionally can include labeled lenses configured to provide wavelength-ratiometric or lifetime-based measurements. Thus, the device and method are configured to take advantage of the most recent polymers for contact lenses and uses their properties to design sensitive and specific ISF-L that bind to the lenses by three different mechanisms, hydrophobic, electrostatic or covalent binding.

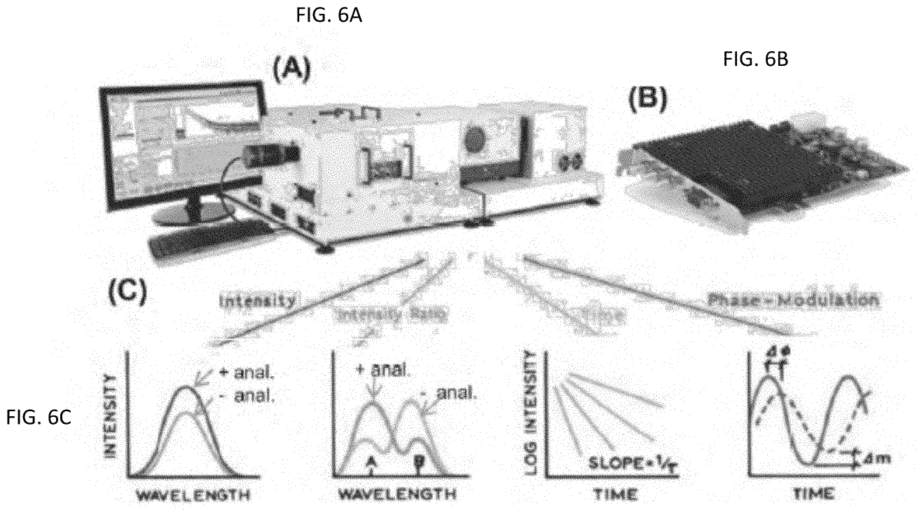

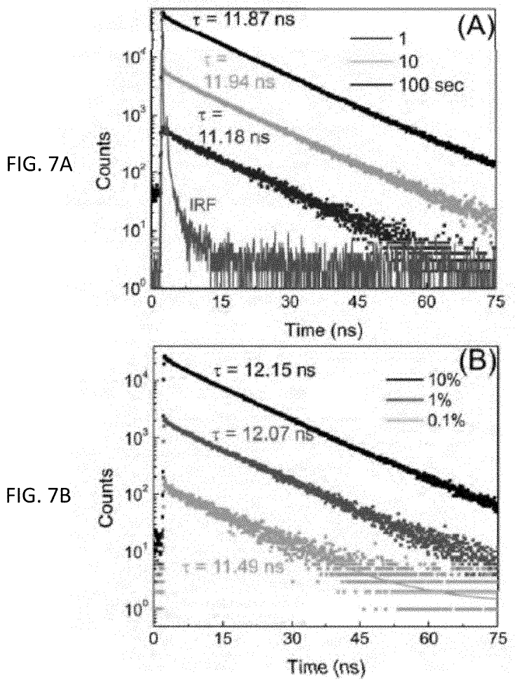

There are two widely used methods to measure intensity decay times. There are correlated single photon counting (TCSPC) and the frequency-domain or phase-modulation method. The instrumentation for these measurements is typically complex, expensive and have not been practical for clinical applications. See FIG. 6A. Fortunately, there have been dramatic improvements in both methods. As an example, the entire electronics for TCSPC can be placed on a single board in a computer. See FIG. 6B. The intensity decays usually result in the same decay time over a 100-fold range of data collection times or excitation light intensities. See FIG. 7. Similar results have been obtained using the frequency-domain method. Confocal optics are not needed with the doubly labeled lenses because the emission from each layer will occur at a different wavelength.

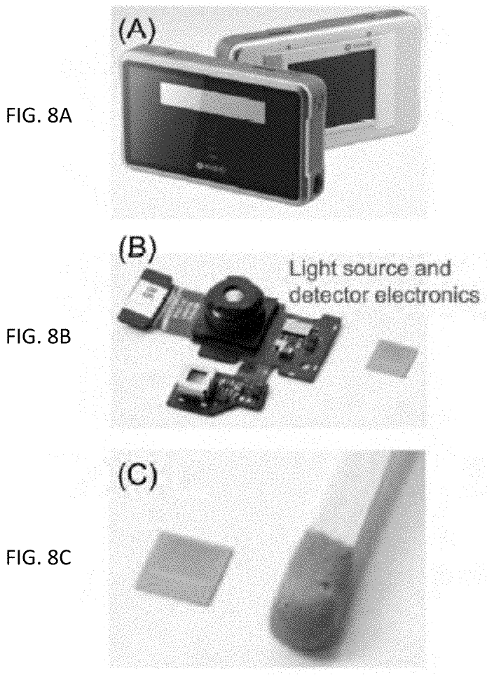

The instrumentation for measuring decay times or decay time imaging can be reduced to a battery powered hand-held device. FIG. 8. There have been advances in all the necessary components. Pulsed solid state lenses or LEDs are available for wavelengths from 260 nm to over 900 nm, which allows excitation of all the commonly used fluorophores. Charge-coupled devices (CCDs) are being rapidly replaced by CMOS detector array (CAD) which require 100-fold less power than CCDs. CDAs are capable of measuring nanosecond (ns) decay times and even useful for fluorescence lifetime imaging microscopy (FLIM). A pulsed solid-state laser and a CMOS detector are now capable of intensity, intensity ratio, intensity decay and even lifetime imaging. CDAs also now can image the time-of-flight from the camera to the surface and back. Since the distance resolution is below 1 inch the CDAs should have time resolution below 1 ns. Another important development is the use of CDAs for identification of individuals by the iris image. Therefore, the software already exists for tracking the iris, which may be needed to image the labeled contact lenses.

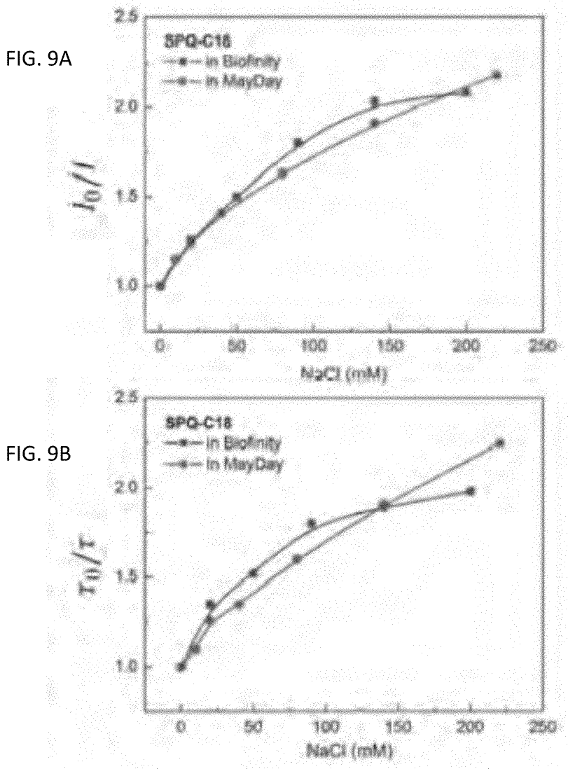

Lenses according to embodiments of the invention also can be used to measure multiple ions. When multiple ISF-L probes are placed in specific locations of a single lens, the probes did not appear to migrate across the lenses by diffusion, even over a period of weeks. See FIG. 9. Additionally, amine-containing fluorophores were attached to SiHG lenses using DC/NHS chemistry as known in the art (data not shown). This linkage was successful because the SiHG lenses contain free carboxyl groups due to the methylacrylic acid used in the polymers.

A favorable property of SiHG lenses is that labeling is possible in several ways, including, but not limited to hydrophobic, electrostatic, or covalent. One method suitable for use in embodiments of the invention is attachment of non-polar side chains (not soluble in water), which bind strongly to SiHG lenses. For electrostatic binding, a fluorophore linked to poly-L-lysine (PL) was used. PL was selected because it is known to bind PDMS, which is frequently used to coat glass and polystyrene surfaces to improve cell adhesion. PL probably binds to contact lenses by both electrostatic and hydrophobic effects. PL and analogs disrupt cell membranes and short sequences containing lysine and arginine bind to and transport other biomolecules across cell membranes. To mimic the ISF-L when bound to contact lenses, a commercially available fluorescein with a hexadecane side chain (Fl-C 16) and fluorescein isothiocyanate (FITC) with covalently linked poly-L-lysine (FL-PL; MW=70-150 kDa) was used.

### 4. EXAMPLES

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: Overview of Probes and Other Materials

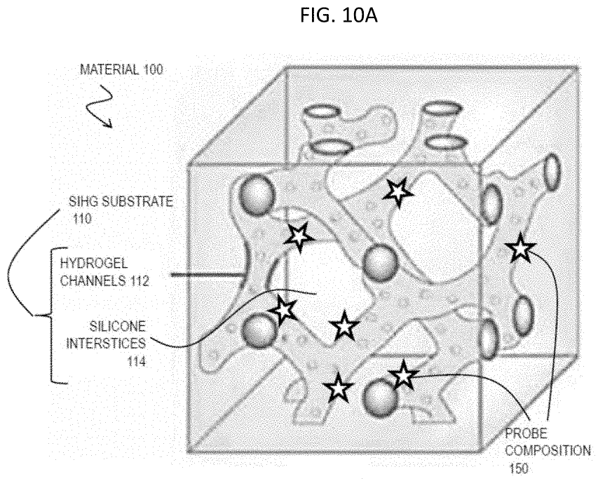

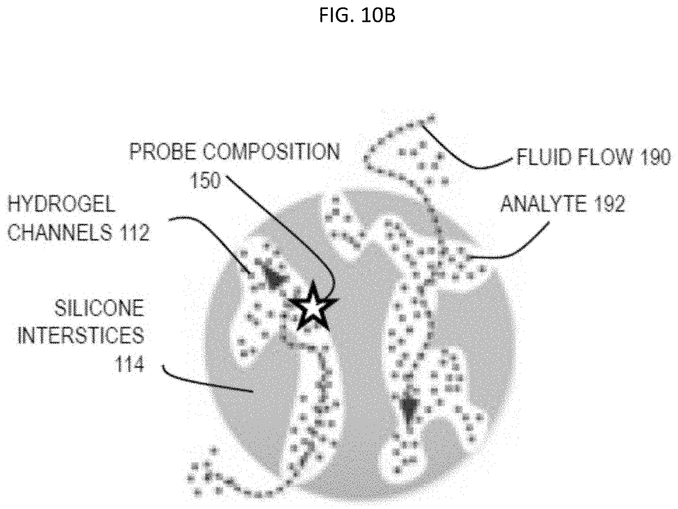

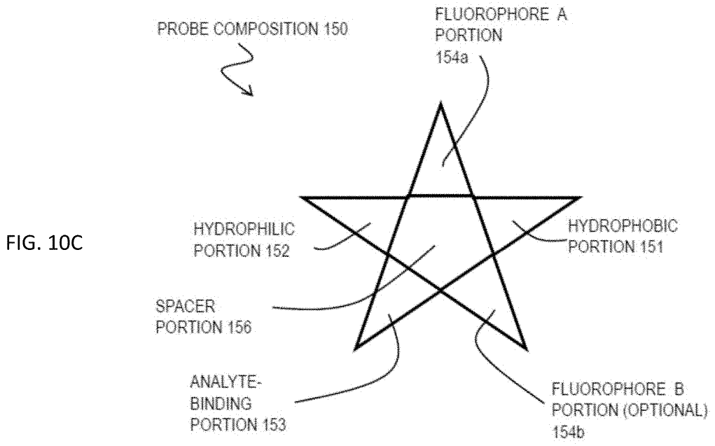

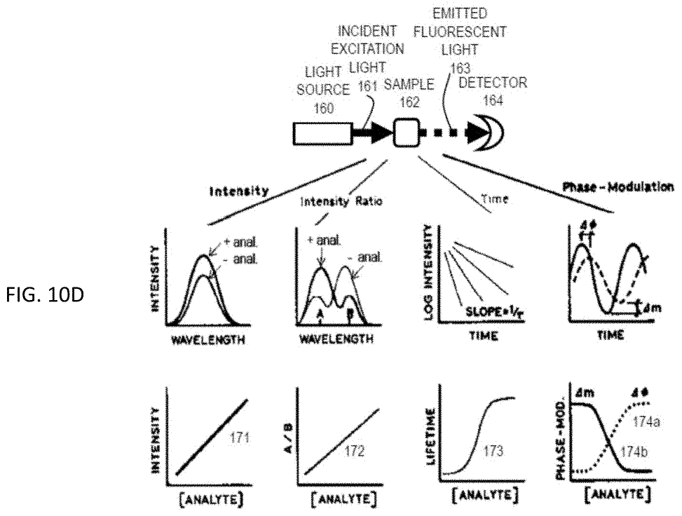

FIG. 10A and FIG. 10B are diagrams that illustrate an example probe-substrate material 100, according to an embodiment. The probe-substrate material 100 (also called material for simplicity hereinafter) includes a SiHG substrate (SiHG) 110 and a probe composition 150 represented by a star symbol, preferably at a concentration that is observable at a remote monitor subsystem when an analyte is present in desirable quantities in a fluid contacting the material. The SiHG substrate 110 inherently includes a network of hydrogel channels 112 that allow the flow of aqueous solutions and a silicone backbone filling the interstices of the network of channels 112, called silicone interstices 114 hereinafter. The hydrogel channels are on the nanoscale, with widths that vary between about 10 nanometers (nm, 1 nm=10⁻⁹ meters) and about 100 nm in some SiHG, and in other SiHG between 5-75 nm. Because this range of channel sizes is smaller than the wavelength of light used in making measurements described herein, there is no scattering of light. The circles and ovals represent the cross sections of the channels 112 as they intersect the faces of a cube of depicted material 100.

There may be many probe compositions 150 in an embodiment, or different probe compositions 150 to detect different analytes, as further described below. Note that each of the probe compositions 150 includes a hydrophilic part and a hydrophobic part so that the probe compositions 150 are found on a boundary (also called interface or silicone-water interface) between the hydrogel channels 112 and the silicone interstices 114. FIG. 10B is a close-up cross section of such an interface, showing a fluid flow 190 represented by the dotted arrows through the hydrogel channels 112 and an analyte 192 within the fluid, each copy of the analyte 192 represented by a dot. Here the silicone interstices 114 are indicated with a gray fill. Note that the depicted probe composition 150 is disposed on the interface between a silicone interstice 114 and the fluid flow 190 and analytes 192 in a hydrogel channel 112. Although shown to illustrate the configuration during operation, the fluid flow 190 and included analyte 192 are not part of the material 100.

FIG. 10C is a block diagram that illustrates an example probe composition 150, according to an embodiment. The probe composition (also called a probe 150) comprises several portions, each portion comprising one or more atoms. The probe 150 includes a hydrophobic (non-polar) portion 151 that prefers to be with the non-polar regions of silicone interstices and a hydrophilic (polar) portion 152 that prefers to be among other polar molecules, such as water in the hydrogel channels 112. Because of this dual attraction, the probe compositions tend to reside at the interface of the hydrogel channels 112 and silicone interstices 114 and not to pass freely though the channels and flush out of the material 100 with the fluid flow 190. This means that the material substrate 110 can be loaded with the probe composition 150 to form material 100; and then the material 100 remains stable as the material 100 is used in a different sample fluid that flow through the hydrogel channels without dislodging many of the probes 150.

For use in an assay, the probe composition also includes at least one analyte-binding portion 153 to capture and bind a molecule of the analyte 192 in the fluid flow 190, if any. In addition, the probe 150 includes at least one fluorophore portion 154a that is configured to change a value of a property of its emitted fluorescent light when an analyte is bound to the analyte-binding portion 153 compared to a value of the property of its emitted fluorescent light when an analyte is not bound to the analyte-binding portion 153. In some embodiments, to ensure that the fluorophore portion 154a is properly spaced from the binding portion 153, a spacer portion 156 is included in the probe 150. In some embodiments, the change in property of fluorescence depends on the interaction of the fluorophore portion 154a with one or more other fluorophores or other functional portions, such as an election donor portion or electron acceptor portion or photon quenching portion or FRET partner portion, called fluorophore B portion 154b hereinafter. In such embodiments, the spacer portion also ensures that the fluorophore B portion 154b is properly spaced from the fluorophore A portion 154a or the analyte-binding portion 153 or both. However, fluorophore B is generally not limited to FRET. For example, fluorophore B portion can also refer to the other part of a PET pair, or a quencher of fluorescence.

FIG. 10D is a set of block diagrams that illustrates example fluorescent light properties that can be measured according to various embodiments. In each embodiment, light 161 of a particular wavelength or wavelength band from a light source 160 is incident on a sample 162, such as material 100 with fluid 190 and analytes 192 therein. Fluorescent light 163 at a different wavelength or band is emitted in response and detected at an optical detector 164 that puts out a digital electrical signal or an analog electrical signal that can be digitized at an analog to digital converter (ADC). Although the emitted fluorescent light 163 is depicted in the same direction as the incident light for purposes of clarity of the diagram, the emitted fluorescent light 163 can be at a different angle than depicted. Example different properties of the emitted fluorescent light 163, among others known in the art, which can be measured include: intensity of the emitted light, represented by the column of graphs on the left; intensity ratio at two or more different wavelengths as indicated by the second column of graphs; intensity decay with time as indicated by the second column of graphs; and phase shift or modulation relative to the incident light. The latter two properties both reflect the lifetime of the emitted fluorescent light 163 after the incident light is turned off, e.g., fluorescent lifetime after a pulse of incident light.

Referring now to the graphs of FIG. 10D, if any of the measurable properties from a probe composition 150 are found to depend on the concentration of the analyte 192 in the fluid 190 for a range of analyte concentrations of interest (e.g., glucose from 40 mg/mL to 300 mg/mL for a diabetic subject), then that probe composition 150 is suitable for forming material 100. The top graph in each column shows examples of different responses for two different concentrations of an analyte, assuming for purposes of illustration that there is a useful dependence of that property on concentration of analyte. The bottom graph in each column depicts calibration curves for each property assuming for purposes of illustration that there is a useful dependence of that property over a useful range of concentrations of analyte.

Still referring to the graphs of FIG. 10D, the top graph on the left column shows that fluorescent intensity forms a peak in a wavelength band at low concentrations of analyte 192 (labeled as “− anal” in the graph). The same graph shows that fluorescent intensity forms a peak in the same wavelength band at high concentrations of analyte (labeled as “+ anal” in the graph). But the graph shows the peak intensity value is greater for the high concentration than for the low concentration. If this relationship were to persist over the analyte concentration range of interest, the bottom graph in the column, with calibration curve 171, would result. Here the intensity of the peak increases with analyte concentration over a concentration range of interest. In this example, the intensity in the wavelength band is the property of the fluorescent light used to determine the concentration of the analyte.

Similarly, and still referring to the graphs of FIG. 10D, the top graph on the second column from the left shows that fluorescent intensity forms peaks in two separate wavelength bands (called band A and band B in the graph) at low concentrations of analyte (labeled as “− anal” in the graph). The intensity of the first peak (band A) is less than the intensity of the second peak (band B). The same graph shows that fluorescent intensity peaks in the same two separate wavelength bands at high concentrations of analyte (labeled as “+ anal” in the graph). But the intensity of the first peak (band A) is greater than the intensity of the second peak (band B). A ratio defined by dividing the intensity of the first peak (band A) by the intensity of the second peak (band B) is lower for low concentration of analyte and higher for the high concentration of analyte. If this relationship were to persist over the analyte concentration range of interest, the bottom graph in the column, with calibration curve 172, would result. Here the ratio of the intensities of the two peaks increases with analyte concentration over a concentration range of interest. In other embodiments, the ratio of the intensities of the two peaks decreases with analyte concentration over a concentration range of interest. In these examples, the ratio of the intensities in the two bands is the property of the fluorescent light used to determine the concentration of the analyte.

As another example from FIG. 10D, the top graph on the third column from the left shows that fluorescent intensity in a particular wavelength band decreases with time. The rate of decay is different for different concentrations of the analyte. The lifetime of the fluorescent response (τ) is given by a reciprocal of a slope of a line in the graph of the log of the intensity in the wavelength band against time. The lifetime 2 is lower for low concentration of analyte and higher for the high concentration of analyte 192. If this relationship were to persist over the analyte concentration range of interest, the bottom graph in the column, with calibration curve 173, would result. Here, the lifetime (τ) increases with analyte concentration over a concentration range of interest. In this example, the lifetime τ is the property of the fluorescent light used to determine the concentration of the analyte.

In a further example from FIG. 10D, the top graph on the fourth column from the left shows electric field changes in time associated with a modulation frequency f. The method uses intensity-modulated light at some modulation frequency f that is much less than the optical frequency. Light modulation frequencies f typically range from 10 megaHertz (MHz, 1 MHz=10⁶ Hertz) to 300 MHz, but can be from 1 MHz to 10 gigaHertz (GHz, 1 GHz=10⁹ Hz); while optical frequencies are in the range of terahertz (THz, 1 THz=10¹² Hertz). The solid curve shows the timing (phase, ϕ) of the measured light modulations relative to that reference beam of light—with successive modulation peaks separated by 2π in phase for a wave period given by 1/f. The graph also shows an amplitude called a modulation for the wave which is related to the intensity. A measured field from emitted fluorescent light is given by the dashed curve and has a slightly different phase Δϕ and modulation Δm from the reference field. If the phase difference Δϕ or the modulation difference Δm from the reference changes with different concentrations of analyte 192, and if either or both were to persist over the analyte concentration range of interest, the bottom graph in the column would result. Here the phase difference Δϕ given by the dotted line increases with analyte concentration over a concentration range of interest, providing calibration curve 174a; and, the modulation difference Δm given by the solid line decreases with analyte concentration over a concentration range of interest, providing calibration curve 174b. In this example, either phase difference Δϕ or modulation difference Δm is the property of the emitted fluorescent light 163 used to determine the concentration of the analyte 192.

### Example 2: Overview of Method

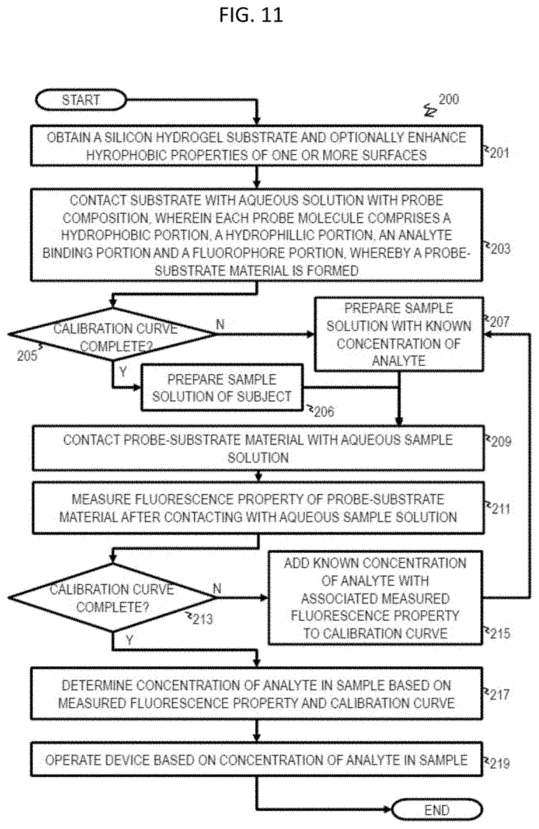

FIG. 11 is a flow chart that illustrates an example method 200 for measuring the concentration of an analyte based on a material, as depicted in FIG. 10A, according to an embodiment. Although steps are depicted in FIG. 11 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 201, a silicone hydrogel substrate is obtained, such as SiHG-CL or a microfluidic device with the silicone hydrogel deposited in one or more sections of one or more channels or in one or more chambers or in one or more reservoirs, or some combination. In some embodiments, the surface of the substrate (e.g., the surfaces of a contact lens) has been treated to make it more hydrophilic, e.g., by oxidation. In some such embodiments, when it is desirable to have probes attach to one or more surfaces of the material instead or in addition to the internal interfaces between hydrogel channels and silicone interstices, the one or more surfaces are treated to enhance the hydrophobic properties of the surface. Examples of such treatment are described in more detail below with reference particular example embodiments. In some embodiments, step 201 includes forming a microfluidic device with the silicone hydrogel deposited in one or more portions of one or more channels or one or more chambers or one or more reservoirs, or some combination.

In step 203, the SiHG substrate is contacted with a aqueous solution with probe compositions 150 for a sufficient duration to obtain a target concentration of the probes in or on the material. The duration can be determined by experimentation for any particular application. As a result, a probe-substrate material 100 is formed. In some embodiments, step 203 involves soaking the material in the solution for the duration. In some embodiments, step 203 involves flushing the material or a microfluidic device including the material with the solution including the probes or otherwise agitating or heating the fluid to achieve the desired concentration of probes in the material within the available duration.

The step following step 205 depends upon whether there is a desired calibration curve for the desired analyte and fluorescent response from the probe, such as one or more of curves 171, 172, 172, 174a or 174b, described above, or some combination. Thus, the method includes a branch point at step 205 to determine whether there is a completed calibration curve or one has to be generated or improved. If there is already a calibration curve of desired reliability that can be used, then control passes directly to step 206 described below. Otherwise, if there is no calibration curve or if an existing calibration curve is lacking in range or statistical convergence, then control passes first to step 207.

In step 207, an aqueous solution with known concentration of analyte is prepared as the sample solution. Any method can be used to produce the known concentration, such as adding a known amount to a known volume of blood or saline or artificial tears to emulate the fluid to be used as a sample from a subject. Control then passes to step 209, described below.

If there is already a calibration curve of desired reliability, then control passes to step 206 instead of step 207. In step 206, an aqueous sample solution is prepared. For example, a blood or urine sample is drawn. In embodiments in which the material is placed in situ, such as a contact lens into the tear fluid coating the subject's eye, then step 206 is omitted and control passes directly to step 209.

In step 209, the probe-substrate material is contacted with the aqueous sample solution (e.g., with known analyte concentration from step 207 or unknown analyte concentration from step 206). For example, in some embodiments the fluid sample is made to flow into a microfluidic device, under gravity, pressure or capillary action, or, in some embodiments, the material or microfluidic device including the material is submerged in the sample. In the example embodiments described below, a contact lens at least part of which is the probe-substrate material is inserted under the eyelids and in front of the cornea of the subject's eye.

In step 211, the calibration curve property of the fluorescent light emitted from the material in contact with the sample fluid is measured in response to excitation by the incident light. The system that excites fluorescence and determines and uses the value of the property related to analyte concentration is called herein a monitor subsystem. Any means known to excite, measure and use the property may be employed as the monitor subsystem, as described in the next section.

After step 211, the next step performed is based on whether there is already an adequate calibration curve, as indicated in step 213 that parallels step 205 described above. If the calibration curve is not complete, then the sample has a known concentration of the analyte and control passes to step 215 to add the measured property of the fluorescent light emitted from the sample and the associated known analyte concentration to the calibration curve. Control then passes back to step 207 to prepare the next sample solution with a known concentration of the analyte. After the loop represented by steps 207, 209, 211 and 215 is repeated enough times, the data converges on a calibration curve or trace that can be used to derive concentration of analyte from measurements of the property of the fluorescent light emitted from a sample with unknown analyte concentration, and the calibration curve is considered complete.

If the calibration curve is complete, then the sample is from a subject and has an unknown concentration of the analyte; and, control passes to step 217. In step 217, the concentration of the analyte in the sample is determined based on the calibration curve and the value of the property of the fluorescent light measured in step 21, described above.

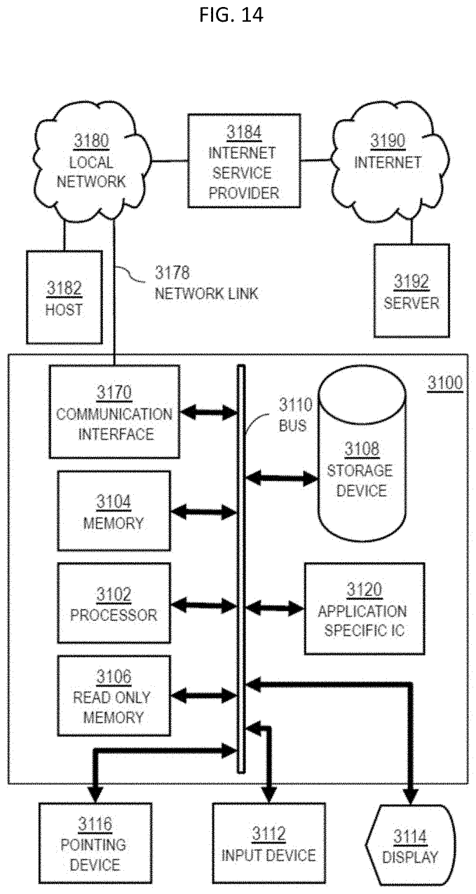

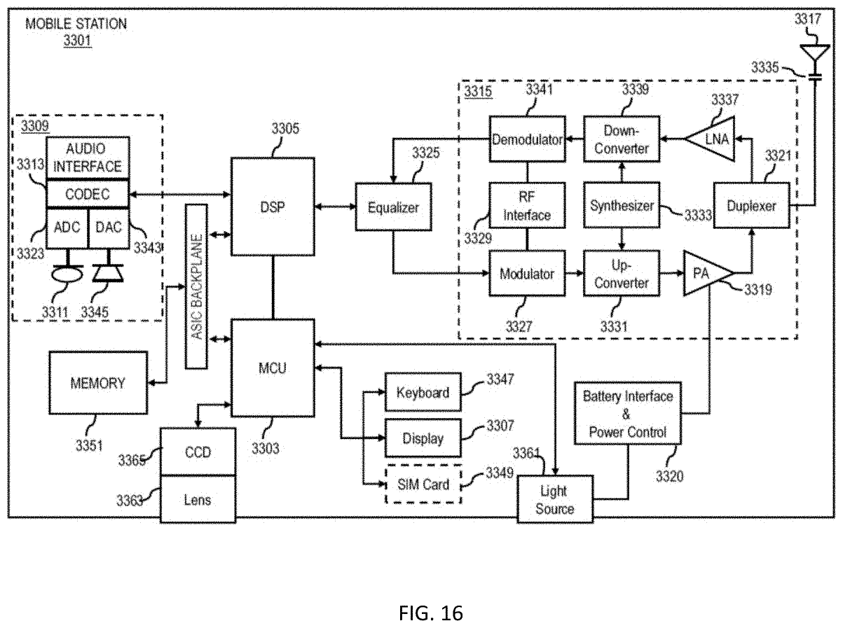

In step 219 a device is operated based on the concentration of the analyte. For example, the value of the concentration of the analyte is presented on a computer or cell phone display device, as described below with reference to FIG. 14 or FIG. 16 (see below). In some embodiments, the value of the concentration is used to determine whether the subject has a condition, such as DED, keratitis, or diabetic hypoglycemia or hyperglycemia; and, to present data indicating the condition on the display device, or operate a device to administer remedial treatment, such as administering insulin to a subject with the condition of diabetic hyperglycemia. The device operated in step 219 is hereinafter called an analyte responsive device, or simply “responsive device.”

### Example 3: Hydrophobic Binding to Lenses

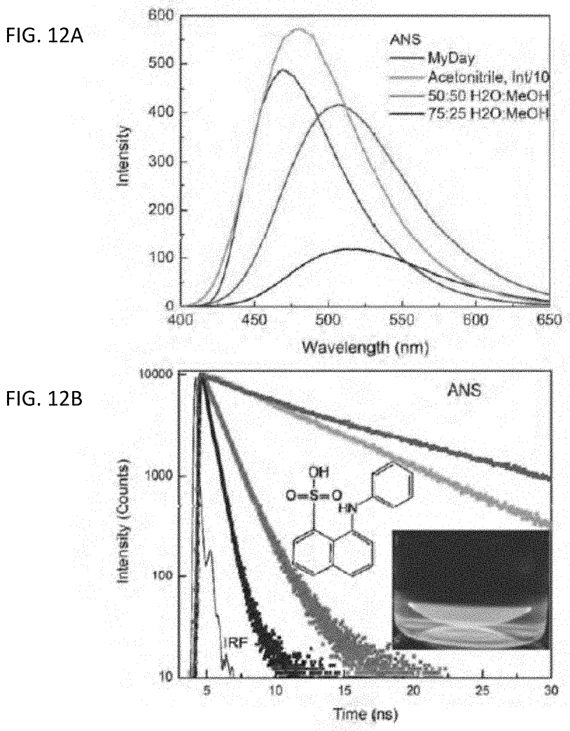

The presence of hydrophobic non-polar regions in SiHG lenses which allow hydrophobic binding to high-silicone Biofinity lenses has been shown. ANS and PRODAN™ display short wavelengths and long lifetime emission in non-polar solvents and a much weaker and longer wave length emission in polar solvents. ANS and PRODAN™ in Biofinity™ lenses displayed emission consistent with a completely non-polar environment. Here, ANS was used to test for non-polar sites in the MyDay™ lenses, which have a much lower silicone content. ANS bound to the MyDay™ lenses with a large increase in intensity (FIG. 12). The short wavelength emission maximum and intensity decay time indicate a very non-polar environment. These results show that fluorophores with hydrophobic side chains are likely to bind to both Biofinity™ and MyDay™ lenses.

### Example 4: 1-Anilino-8-Naphthalene Sulfonic Acid Binding to Lenses

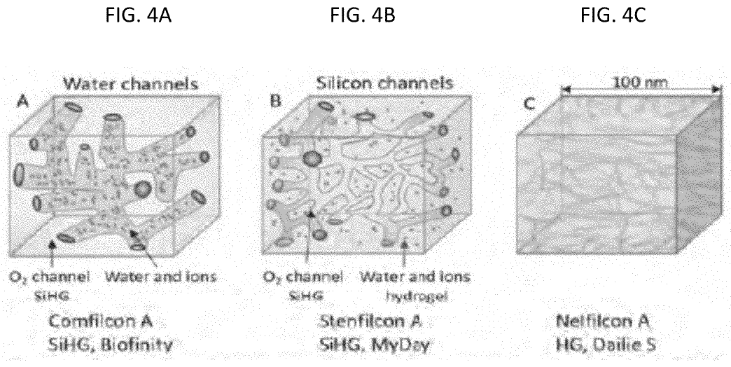

The schematics in FIG. 4 suggested the presence of a water-hydrophobic interface region. In the field of fluorescence spectroscopy there is a long history of detecting hydrophobic regions of biomolecules with solvent or polarity-sensitive fluorophores. In a previous publication, the existence of such regions in the silicone-rich Biofinity™ lenses was demonstrated. However, it was not known if similar regions existed in the low silicon MyDay™ lenses. The existence of hydrophobic regions was important because in this study, it was planned to bind ISF to long alkyl chains for binding to lenses. Testing for hydrophobic sites was performed using 1-anilino-8-naphthalene sulfonic acid (ANS) which is known to be quenched in water and to be highly fluorescent in non-polar locations. Equivalent amounts of ANS were added to lens solvents and water-containing solvents (FIG. 12). ANS bound readily to the MyDay™ lenses, and displayed bright emission. The emission was weaker with a longer wavelength emission maximum in more polar solvents. The fluorescence lifetime of ANS displayed the largest value when bound to the MyDay lenses. The ANS spectra and lifetime are consistent with a completely non-polar environment. Similar results with the polarity-sensitive probe PRODAN in Biofinity™ lenses, which suggests to us that non-polar region, for binding of hydrophobic ISF, will be present in most SiHG lenses.

The in-plane x-y resolution is more than adequate for imaging x-y planes of the tear films. Results shown in FIG. 3 show that the z-resolution with the 20× objective is adequate for separate detection of the PLTF and the PoLTF. See the effective observed volume (blue ovals) placed on the contact lens and cornea in FIG. 3. If needed, the z-resolution can be increased using a higher NA objective. For example, using a 100×, NA=0.90 air objective, the resolution values were dxy=373 nm and dz=2.0 μm. Higher resolution can be obtained using immersion objectives. The 60×, NA=1.4 immersion objective had a z-resolution of 0.84 μm. This spatial resolution allows separate observations of emission from the PLTF and PoLTF. When the lenses have two different fluorophores each probe can be observed at a different wavelength without confocal detection.

### Example 5: Surface Localization of Fluorophores

In the previous example, the ANS was most likely distributed uniformly throughout the lens. However, our goal is to obtain surface-localized ISF. The thickness of the cornea and contact lenses allows the distribution through the lens (z-axis) to be measured with laser scanning confocal fluorescence microscopy (LSCFM). Contact lenses have central thickness from 70 to 200 μm. The thickness of the PoLTF is 3-8 μm. The PLTF is thinner and its thickness is less certain. When providing tools to measure the ion concentrations in these films, the ability to measure the surface localization of fluorophore depends on the z-axis (axial) resolution of the instrument. In confocal microscopy, the xy-plane and z-axial resolution are given by dxy=1.22A/2NA and dz=2A·n/(NA)² where A is the wavelength, n is the refractive index and NA is the numerical aperture of the objective. Here, a 20×, NA=0.40 objective, and N=1.5, at 550 nm were used. The diffraction limited resolutions were dxy=839 nm and dz=10.2 μm.

### Example 6: Overview of Systems

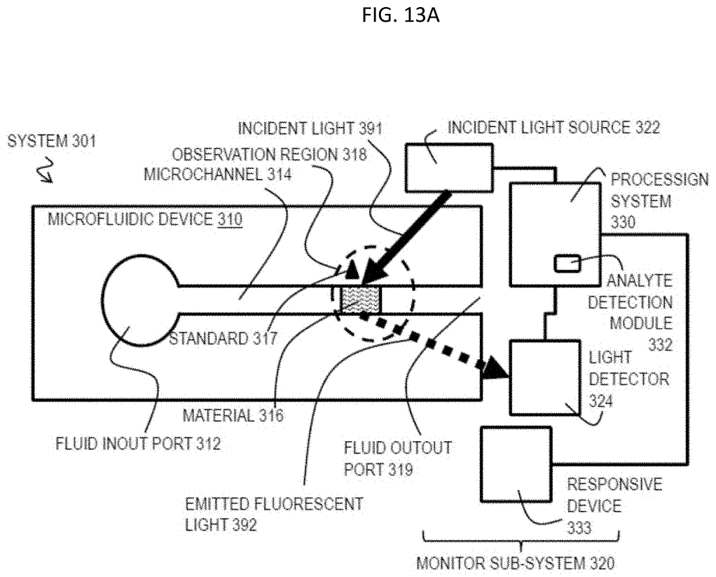

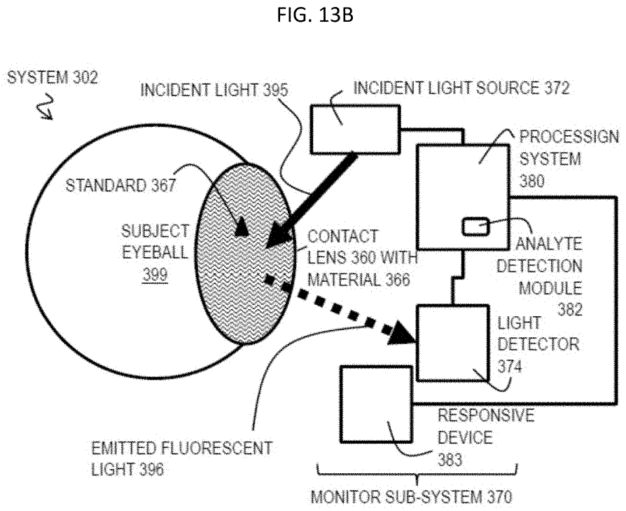

FIG. 13A and FIG. 13B are block diagrams that illustrate example systems that detect the concentration of an analyte in fluid from a subject, according to some embodiments. FIG. 13A depicts an example system using a microfluidic device into which a sample solution is introduced and FIG. 13B depicts an example system using a contact lens placed in situ in the tear fluid of a subject's eye in front of the cornea and behind the closed eyelid.

FIG. 13A is a block diagram that illustrates an example microfluidic system 301 that detects concentration of an analyte in fluid from a subject. According to some embodiments, the system 301 includes a microfluidic device 310 and a monitor subsystem 320. The microfluidic device includes a non-hydrogel substrate, such as glass or PDMS into which is formed, by etching or injection molding or other known process, a fluid input port 312, a microchannel 314 having both width and depth in the range from 1-1000 μm, and a fluid output port 319, in fluid communication with a chamber or other basin or waste disposal (not shown). In a section called an observation region 318, a probe-substrate material 316, such as material 100, is disposed. In some embodiments, e.g. in some embodiments based on measuring fluorescence intensity with a calibration curve like 171, it is advantageous to include a standard 317 that has a known intensity in the fluorescent band of wavelengths. During operation, a sample solution is introduced at fluid input 312, flows through microchannel 324 and encounters the probe-substrate material 316.

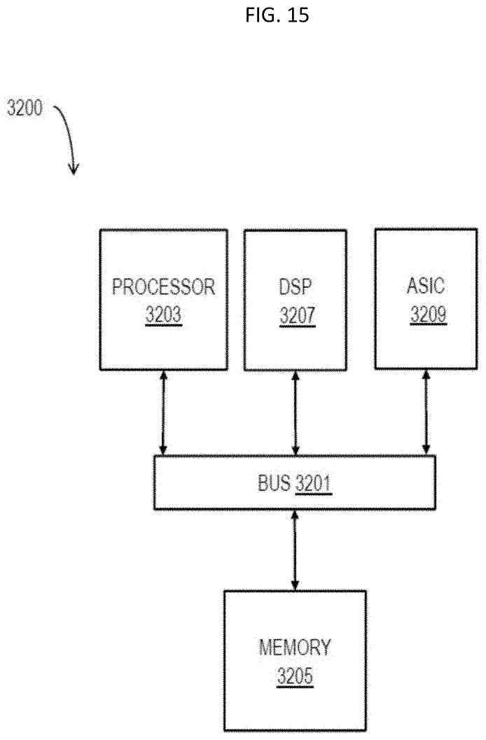

The monitor sub-system 320 provides the excitation light and makes the fluorescence measurements and determines the concentration of the analyte in the fluid passing through the material 316. To avoid contaminating the sample fluid, it is advantageous if no component of the monitor sub-system contacts the fluid in the microfluidic device 310. The monitor sub-system 320 includes incident light source 322, light detector 324, processing system 330 and responsive device 333. An analyte detection module 332 is implemented as hardware, firmware or software, or some combination, in processing system 330 to operate the processing system 330 and light source 322 and detector 324 and responsive device 333 as described in the method 200 of FIG. 11. Although incident light 391 and emitted fluorescent light 392 are depicted to illustrate operation of the subsystem, they are not part of the sub-system except during operation. In the illustrated embodiment, the processing system 330 is in communication with the incident light source 322, light detector 324 and responsive device 333 through one or more wired or wireless connections. In some embodiments, one or more of these components are included in a computer system as depicted in FIG. 14, chip set as depicted in FIG. 15, or mobile terminal or cell phone as depicted in FIG. 16.

The monitor sub-system 320, as noted above and depicted in FIGS. 14, 15, and 16, includes incident light source 322, light detector 324, processing system 330 and responsive device 333. The light source 322 includes a light source, such as a laser, light emitting diode (LED), pulsed laser diode (LD), UV LED, incandescent lamp, fluorescent lamp, including any ultraviolet (UV) source, and any optical couplers used to condition the light (e.g., to polarize, filter, modify wavelength, modify amplitude, modify phase or otherwise delay) and direct the light onto the material 316. Optical couplers include one or more of an optical filter, a polarization controller, an optical amplifier, a frequency doubler, injection locking, fiber-optical circulator, fiber coupler, and free-space optical components (e.g., mirrors, lenses, polarizers, open space, vacuum space, etc.) individually or collectively. The light detector 324 includes one or more optical couplers and single, paired or one or two dimensional arrays of single or paired detectors that output analog or digital electrical signals, along with any analog to digital converter (ADS), filters or other electronic components useful to condition the electrical data for processing by the processing system 330. The processing system includes one or more of the computer system depicted in FIG. 14, chip set depicted in FIG. 15, or smart cell phone depicted in FIG. 16. The analyte detection module 332 is configured to: operate the light source 322 to produce desired properties of the incident light to direct onto the material 316, including operating any motors or actuators to point or tune the light; to determine the duration and frequency of measurements to be made at the detector 324; to condition the data after the data is received from the detector 324, including determining the property of the fluorescent light; to use or construct the calibration curve; to determine the analyte concentration; and to operate the responsive device 333 based on the value of the concentration of the analyte.

FIG. 13B is a block diagram that illustrates an example in situ system 302 that detects concentration of an analyte in fluid from a subject. According to some embodiments, the system 302 includes a monitor sub-system much as described above for system 301 and, instead of a microfluidic device 310, a contact lens 360 with at least a part including a probe-substrate material 366. For example, in some embodiments based on measuring fluorescence intensity with a calibration curve like 171, it is advantageous to include a standard 367 that has a known intensity in the fluorescent band of wavelengths. During operation, the contact lens 360 is inserted in front of a cornea of an eyeball 399 of a subject typical of contact lens locations when the eyelids are closed. In situ tear fluid makes up the sample solution. The monitor sub-system 370 is made up of an incident light source 372, light detector 374, processing system 380, responsive device 383 and analyte detection module 382 configured for operation with the material 366 of contact lens 360, but otherwise is analogous to those items 320, 322, 324, 330, 333 and 332, respectively, described above. Similarly, the incident light 395 and emitted fluorescent light 396 are particular to the material 366 of contact lens 360, but otherwise are analogous to the incident light 391 and emitted fluorescent light 392 described above.

Although processes, equipment, and data structures are depicted in FIG. 13A and FIG. 13B as integral blocks in a particular arrangement for purposes of illustration, in other embodiments one or more processes or data structures, or portions thereof, are arranged in a different manner, on the same or different hosts, in one or more databases, or are omitted, or one or more different processes or data structures are included on the same or different hosts. For example, in some embodiments, the light source 322 is in autonomous mode and operated independently of the processing system 330, so that the communication connection between them is omitted.

### Example 7: Computational Hardware Overview

FIG. 14 illustrates a computer system 3100 upon which an embodiment of the invention may be implemented. Computer system 3100 includes a communication mechanism such as a bus 3110 for passing information between other internal and external components of the computer system 3100. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit).). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 3100, or a portion thereof, constitutes a means for performing one or more steps of one or more methods described herein.

A sequence of binary digits constitutes digital data that is used to represent a number or code for a character. A bus 3110 includes many parallel conductors of information so that information is transferred quickly among devices coupled to the bus 3110. One or more processors 3102 for processing information are coupled with the bus 3110. A processor 3102 performs a set of operations on information. The set of operations include bringing information in from the bus 3110 and placing information on the bus 3110. The set of operations also typically include comparing two or more units of information, shifting positions of units of information, and combining two or more units of information, such as by addition or multiplication. A sequence of operations to be executed by the processor 3102 constitutes computer instructions.

Computer system 3100 also includes a memory 3104 coupled to bus 3110. The memory 3104, such as a random access memory (RAM) or other dynamic storage device, stores information including computer instructions. Dynamic memory allows information stored therein to be changed by the computer system 3100. RAM allows a unit of information stored at a location called a memory address to be stored and retrieved independently of information at neighboring addresses. The memory 3104 is also used by the processor 3102 to store temporary values during execution of computer instructions. The computer system 3100 also includes a read only memory (ROM) 3106 or other static storage device coupled to the bus 3110 for storing static information, including instructions, that is not changed by the computer system 3100. Also coupled to bus 3110 is a non-volatile (persistent) storage device 3108, such as a magnetic disk or optical disk, for storing information, including instructions, that persists even when the computer system 3100 is turned off or otherwise loses power.

Information, including instructions, is provided to the bus 3110 for use by the processor from an external input device 3112, such as a keyboard containing alphanumeric keys operated by a human user, or a sensor. A sensor detects conditions in its vicinity and transforms those detections into signals compatible with the signals used to represent information in computer system 3100. Other external devices coupled to bus 3110, used primarily for interacting with humans, include a display device 3114, such as a cathode ray tube (CRT) or a liquid crystal display (LCD), for presenting images, and a pointing device 3116, such as a mouse or a trackball or cursor direction keys, for controlling a position of a small cursor image presented on the display 3114 and issuing commands associated with graphical elements presented on the display 3114.

In the illustrated embodiment, special purpose hardware, such as an application specific integrated circuit (IC) 3120, is coupled to bus 3110. The special purpose hardware is configured to perform operations not performed by processor 3102 quickly enough for special purposes. Examples of application specific ICs include graphics accelerator cards for generating images for display 3114, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 3100 also includes one or more instances of a communications interface 3170 coupled to bus 3110. Communication interface 3170 provides a two-way communication coupling to a variety of external devices that operate with their own processors, such as printers, scanners and external disks. In general, the coupling is with a network link 3178 that is connected to a local network 3180 to which a variety of external devices with their own processors are connected. For example, communication interface 3170 may be a parallel port or a serial port or a universal serial bus (USB) port on a personal computer. In some embodiments, communications interface 3170 is an integrated services digital network (ISDN) card or a digital subscriber line (DSL) card or a telephone modem that provides an information communication connection to a corresponding type of telephone line. In some embodiments, a communication interface 3170 is a cable modem that converts signals on bus 3110 into signals for a communication connection over a coaxial cable or into optical signals for a communication connection over a fiber optic cable.

As another example, communications interface 3170 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 3170 sends and receives electrical, acoustic or electromagnetic signals, including infrared and optical signals that carry information streams, such as digital data.

The term computer-readable medium is used herein to refer to any medium that participates in providing information to processor 3102, including instructions for execution. Such a medium may take many forms, including, but not limited to, non-volatile media, volatile media and transmission media. Non-volatile media include, for example, optical or magnetic disks, such as storage device 3108. Volatile media include, for example, dynamic memory 3104. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 3102, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 3102, except for carrier waves and other signals.

Logic encoded in one or more tangible media includes one or both of processor instructions on a computer-readable storage media and special purpose hardware, such as ASIC 313120.

Network link 3178 typically provides information communication through one or more networks to other devices that use or process the information. For example, network link 3178 may provide a connection through local network 3180 to a host computer 3182 or to equipment 3184 operated by an Internet Service Provider (ISP). ISP equipment 3184 in turn provides data communication services through the public, world-wide packet-switching communication network of networks now commonly referred to as the Internet 3190. A computer called a server 3192 connected to the Internet provides a service in response to information received over the Internet. For example, server 3192 provides information representing video data for presentation at display 3114.

The invention is related to the use of computer system 3100 for implementing the techniques described herein. According to one embodiment of the invention, those techniques are performed by computer system 3100 in response to processor 3102 executing one or more sequences of one or more instructions contained in memory 3104. Such instructions, also called software and program code, may be read into memory 3104 from another computer-readable medium such as storage device 3108. Execution of the sequences of instructions contained in memory 3104 causes processor 3102 to perform the method steps described herein. In alternative embodiments, hardware, such as application specific integrated circuit 3120, may be used in place of or in combination with software to implement the invention. Thus, embodiments of the invention are not limited to any specific combination of hardware and software.

The signals transmitted over network link 3178 and other networks through communications interface 3170, carry information to and from computer system 3100. Computer system 3100 can send and receive information, including program code, through the networks 3180, 3190 among others, through network link 3178 and communications interface 3170. In an example using the Internet 3190, a server 3192 transmits program code for a particular application, requested by a message sent from computer 3100, through Internet 3190, ISP equipment 3184, local network 3180 and communications interface 3170. The received code may be executed by processor 3102 as it is received or may be stored in storage device 3108 or other non-volatile storage for later execution, or both. In this manner, computer system 3100 may obtain application program code in the form of a signal on a carrier wave.

Various forms of computer readable media may be involved in carrying one or more sequence of instructions or data or both to processor 3102 for execution. For example, instructions and data may initially be carried on a magnetic disk of a remote computer such as host 3182. The remote computer loads the instructions and data into its dynamic memory and sends the instructions and data over a telephone line using a modem. A modem local to the computer system 3100 receives the instructions and data on a telephone line and uses an infra-red transmitter to convert the instructions and data to a signal on an infra-red a carrier wave serving as the network link 3178. An infrared detector serving as communications interface 3170 receives the instructions and data carried in the infrared signal and places information representing the instructions and data onto bus 3110. Bus 3110 carries the information to memory 3104 from which processor 3102 retrieves and executes the instructions using some of the data sent with the instructions. The instructions and data received in memory 3104 may optionally be stored on storage device 3108, either before or after execution by the processor 3102.

FIG. 15 illustrates a chip set 3200 upon which an embodiment of the invention may be implemented. Chip set 3200 is programmed to perform one or more steps of a method described herein and includes, for instance, the processor and memory components described with respect to FIG. 3131 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 3200, or a portion thereof, constitutes a means for performing one or more steps of a method described herein.

In one embodiment, the chip set 3200 includes a communication mechanism such as a bus 3201 for passing information among the components of the chip set 3200. A processor 3203 has connectivity to the bus 3201 to execute instructions and process information stored in, for example, a memory 3205. The processor 3203 may include one or more processing cores with each core configured to perform independently. A multi-core processor enables multiprocessing within a single physical package. Examples of a multi-core processor include two, four, eight, or greater numbers of processing cores. Alternatively, or in addition, the processor 3203 may include one or more microprocessors configured in tandem via the bus 3201 to enable independent execution of instructions, pipelining, and multithreading. The processor 3203 may also be accompanied with one or more specialized components to perform certain processing functions and tasks such as one or more digital signal processors (DSP) 3207, or one or more application-specific integrated circuits (ASIC) 3209. A DSP 3207 typically is configured to process real-world signals (e.g., sound) in real time independently of the processor 3203. Similarly, an ASIC 3209 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 3203 and accompanying components have connectivity to the memory 3205 via the bus 3201. The memory 3205 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 3205 also stores the data associated with or generated by the execution of one or more steps of the methods described herein.

FIG. 16 is a diagram of exemplary components of a mobile terminal 3300 (e.g., cell phone handset) for communications, which is capable of operating in the system, according to one embodiment. In some embodiments, mobile terminal 3301, or a portion thereof, constitutes a means for performing one or more steps described herein. Generally, a radio receiver is often defined in terms of front-end and back-end characteristics. The front-end of the receiver encompasses all of the Radio Frequency (RF) circuitry whereas the back-end encompasses all of the base-band processing circuitry.

As used in this application, the term “circuitry” refers to both: (1) hardware-only implementations (such as implementations in only analog and/or digital circuitry), and (2) to combinations of circuitry and software (and/or firmware) (such as, if applicable to the particular context, to a combination of processor(s), including digital signal processor(s), software, and memory(ies) that work together to cause an apparatus, such as a mobile phone or server, to perform various functions). This definition of “circuitry” applies to all uses of this term in this application, including in any claims. As a further example, as used in this application and if applicable to the particular context, the term “circuitry” would also cover an implementation of merely a processor (or multiple processors) and its (or their) accompanying software/or firmware. The term “circuitry” would also cover if applicable to the particular context, for example, a baseband integrated circuit or applications processor integrated circuit in a mobile phone or a similar integrated circuit in a cellular network device or other network devices.

Pertinent internal components of the telephone include a Main Control Unit (MCU) 3303, a Digital Signal Processor (DSP) 3305, and a receiver/transmitter unit including a microphone gain control unit and a speaker gain control unit. A main display unit 3307 provides a display to the user in support of various applications and mobile terminal functions that perform or support the steps as described herein. The display 3307 includes display circuitry configured to display at least a portion of a user interface of the mobile terminal (e.g., mobile telephone). Additionally, the display 3307 and display circuitry are configured to facilitate user control of at least some functions of the mobile terminal. An audio function circuitry 3309 includes a microphone 3311 and microphone amplifier that amplifies the speech signal output from the microphone 3311. The amplified speech signal output from the microphone 3311 is fed to a coder/decoder (CODEC) 3313.

A radio section 3315 amplifies power and converts frequency in order to communicate with a base station, which is included in a mobile communication system, via antenna 3317. The power amplifier (PA) 3319 and the transmitter/modulation circuitry are operationally responsive to the MCU 3303, with an output from the PA 3319 coupled to the duplexer 3321 or circulator or antenna switch, as known in the art. The PA 3319 also couples to a battery interface and power control unit 3320.

In use, a user of mobile terminal 3301 speaks into the microphone 3311 and his or her voice along with any detected background noise is converted into an analog voltage. The analog voltage is then converted into a digital signal through the Analog to Digital Converter (ADC) 3323. The control unit 3303 routes the digital signal into the DSP 3305 for processing therein, such as speech encoding, channel encoding, encrypting, and interleaving. In one embodiment, the processed voice signals are encoded, by units not separately shown, using a cellular transmission protocol such as enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wideband code division multiple access (WCDMA), wireless fidelity (WiFi), satellite, and the like, or any combination thereof.

The encoded signals are then routed to an equalizer 3325 for compensation of any frequency-dependent impairments that occur during transmission though the air such as phase and amplitude distortion. After equalizing the bit stream, the modulator 3327 combines the signal with a RF signal generated in the RF interface 3329. The modulator 3327 generates a sine wave by way of frequency or phase modulation. In order to prepare the signal for transmission, an up-converter 3331 combines the sine wave output from the modulator 3327 with another sine wave generated by a synthesizer 3333 to achieve the desired frequency of transmission. The signal is then sent through a PA 3319 to increase the signal to an appropriate power level. In practical systems, the PA 3319 acts as a variable gain amplifier whose gain is controlled by the DSP 3305 from information received from a network base station. The signal is then filtered within the duplexer 3321 and optionally sent to an antenna coupler 3335 to match impedances to provide maximum power transfer. Finally, the signal is transmitted via antenna 3317 to a local base station. An automatic gain control (AGC) can be supplied to control the gain of the final stages of the receiver. The signals may be forwarded from there to a remote telephone which may be another cellular telephone, any other mobile phone or a landline connected to a Public Switched Telephone Network (PSTN), or other telephony networks.

Voice signals transmitted to the mobile terminal 3301 are received via antenna 3317 and immediately amplified by a low noise amplifier (LNA) 3337. A down-converter 3339 lowers the carrier frequency while the demodulator 3341 strips away the RF leaving only a digital bit stream. The signal then goes through the equalizer 3325 and is processed by the DSP 3305. A Digital to Analog Converter (DAC) 3343 converts the signal and the resulting output is transmitted to the user through the speaker 3345, all under control of a Main Control Unit (MCU) 3303 which can be implemented as a Central Processing Unit (CPU) (not shown).

The MCU 3303 receives various signals including input signals from the keyboard 3347. The keyboard 3347 and/or the MCU 3303 in combination with other user input components (e.g., the microphone 3311) comprise a user interface circuitry for managing user input. The MCU 3303 runs a user interface software to facilitate user control of at least some functions of the mobile terminal 3301 as described herein. The MCU 3303 also delivers a display command and a switch command to the display 3307 and to the speech output switching controller, respectively. Further, the MCU 3303 exchanges information with the DSP 3305 and can access an optionally incorporated SIM card 3349 and a memory 3351. In addition, the MCU 3303 executes various control functions required of the terminal. The DSP 3305 may, depending upon the implementation, perform any of a variety of conventional digital processing functions on the voice signals. Additionally, DSP 3305 determines the background noise level of the local environment from the signals detected by microphone 3311 and sets the gain of microphone 3311 to a level selected to compensate for the natural tendency of the user of the mobile terminal 3301.

The CODEC 3313 includes the ADC 3323 and DAC 3343. The memory 3351 stores various data including call incoming tone data and is capable of storing other data including music data received via, e.g., the global Internet. The software module could reside in RAM memory, flash memory, registers, or any other form of writable storage medium known in the art. The memory device 3351 may be, but not limited to, a single memory, CD, DVD, ROM, RAM, EEPROM, optical storage, magnetic disk storage, flash memory storage, or any other non-volatile storage medium capable of storing digital data.

An optionally incorporated SIM card 3349 carries, for instance, important information, such as the cellular phone number, the carrier supplying service, subscription details, and security information. The SIM card 3349 serves primarily to identify the mobile terminal 3301 on a radio network. The card 3349 also contains a memory for storing a personal telephone number registry, text messages, and user specific mobile terminal settings.

In some embodiments, the mobile terminal 3301 includes a digital camera comprising an array of optical detectors, such as charge coupled device (CCD) array 3365. The output of the array is image data that is transferred to the MCU for further processing or storage in the memory 3351 or both. In the illustrated embodiment, the light impinges on the optical array through a lens 3363, such as a pin-hole lens or a material lens made of an optical grade glass or plastic material. In the illustrated embodiment, the mobile terminal 3301 includes a light source 3361, such as a LED to illuminate a subject for capture by the optical array, e.g., CCD 3365. The light source is powered by the battery interface and power control module 3320 and controlled by the MCU 3303 based on instructions stored or loaded into the MCU 3303.

### Example 8: Intensity-Independent Fluorescence Measurements

When the z-axis spatial distributions of the fluorophores are known the labeled lenses can be tested using a time-resolved spectrofluorometer. The excitation in these embodiments is from a pulsed light source and collection of the emission is accomplished using an optical fiber. This same configuration can be used for the ex vivo and in vivo experiments with rabbits. The dominant emission is expected to be from the labeled lenses with different emission spectra, so confocal optics will not be needed. A simple instrument can be affordable to laboratories and in clinical practice. Fiber optic microscopes and endoscopes have been developed using single-mode and multi-mode fibers, and GRIN lenses. Some of these instruments are hand-held for use during medical examinations. Presently available electronics and imaging electronics allow hand-held devices or cell phone-size devices for measurements of the ISF-labeled lenses.

It is difficult to use absolute intensities for fluorescence sensing. The intensity values can vary greatly depending on the specific contact lens cornea sample and on the precise optical geometry. To circumvent this problem, the ISF-L probes is designed to provide ion concentration information based on either wavelength-ratiometric or lifetime-based sensing. These measurements are independent of the total intensity. Intensity measurements alone can be changed due to numerous factors, such as blinking or patient movement. If the probe displays a spectral shift in response to ions, the intensity ratios from two different emission wavelengths remain the same unless there is significant background emission. If the probe displays a change in lifetime, this also is independent of intensity. In this laboratory, both the time-domain (TD) and the frequency-domain (FD), also called phase-modulation method can be used. The FD measurements can be performed in the presence of significant room light which provides only a constant background signal that does not affect the phase angle measurements.

### Example 9: Example Analyte Embodiments

Recent developments in silicone hydrogel contact lens (SiHG-CL) and fluorescent probes provide an opportunity to perform measurements of electrolyte concentrations with specially labeled (probe composition-infused) contact lenses for detection of dry eye disease. One of these developments, as mentioned earlier in this disclosure, is the existence of hydrogel channels or nanochannels (silicone interstices) within the silicone hydrogel allowing the flow of aqueous solutions through the lens. Certain of the embodiments disclosed below include probe compositions that may be anchored within the silicone interstices yet still have a portion exposed to aqueous flow through these nanochannels useful for measuring one or more analytes. Note that one of the analytes 192 which may be measured is glucose, and these embodiments are discussed more fully herein.

The two main forms of dry eye disease are aqueous deficient dry eye (ADDE) and Evaporative dry eye (EDE). ADDE and EDE are highly correlated with an electrolyte imbalance in tears. High electrolyte concentrations is regarded as the most accurate and objective biomarker for DED. For this reason, probe compositions 150 have been developed for detecting various electrolyte concentrations for use in a SiHG-CL.

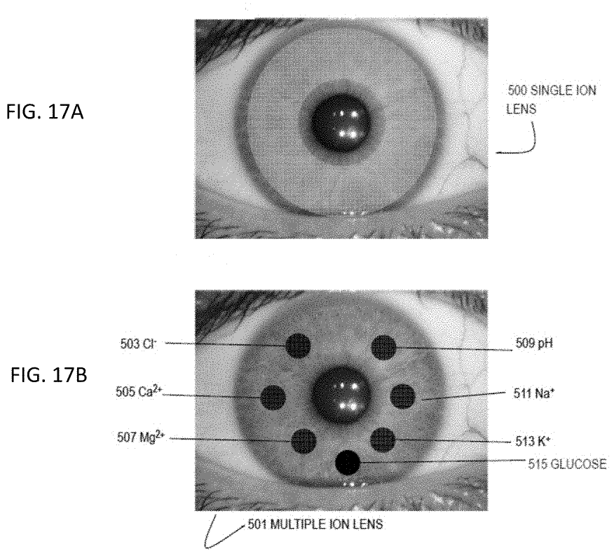

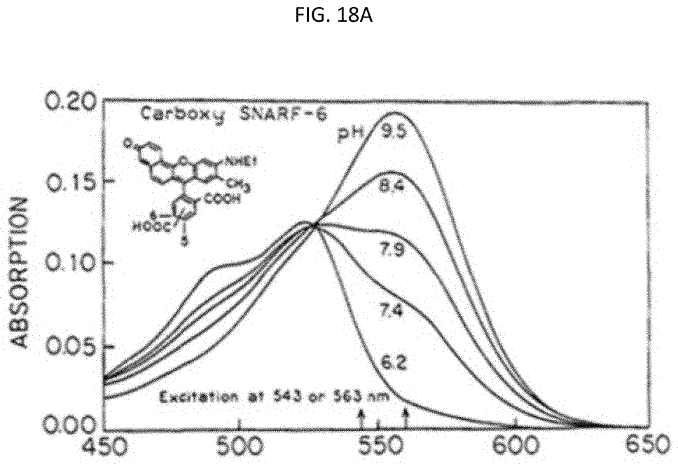

FIG. 17A and FIG. 17B are images that illustrate a contact lens that can be crafted using materials containing probe compositions to detect one or more various analytes such as the ones shown, including glucose, according to various embodiments. A multiple ion lens is one of the disclosed embodiments herein, and it uses a material incorporating various probe compositions disclosed herein such that the concentrations of one or more of the following electrolytes can be detected: pH (concentration of H⁺) using probe composition 509, Na⁺ using probe composition 511, K⁺ using probe composition 513, Ca²⁺ using probe composition 505, Mg²⁺ using probe composition 507, Cl⁻ using probe composition 503, and glucose using probe composition 515. A lens with the ability to detect one or more of these electrolytes may be constructed using a material having embedded therein probe compositions 150 designed to detect the concentration of these electrolytes.

### Example 10: SNARF Probe Compositions Useful for Measuring pH

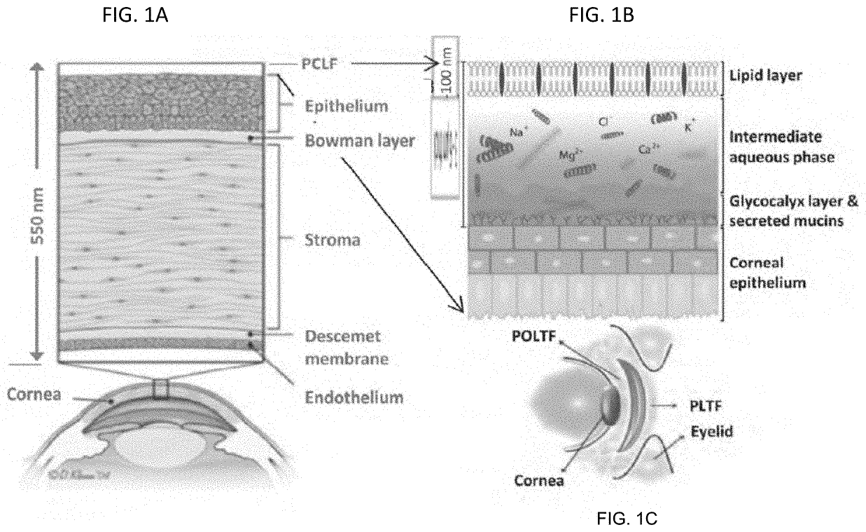

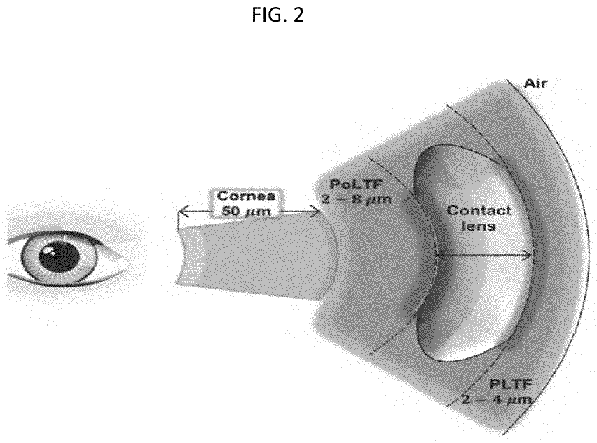

As mentioned in the discussion of FIG. 1C, embodiments of probe compositions 150 disclosed for measuring pH levels in tears include at least a hydrophilic portion 152, hydrophobic portion 151, an analyte binding portion 153.

Embodiments of probes for use in SiHG-CLs can include known pH probes of the seminaphto-fluorescein (SNAFL) and seminaphtorhodafluors (SNARF) series. SNARF and SNAFL probes display changes in their absorption and emission spectra and also display changes in lifetime on pH-induced ionization. Embodiments are disclosed of probe compositions having hydrophobic alkyl side chains attached to or in chemical communication with SNARF-based probes capable of binding to the silicone rich region of the lenses. The SNARF family probes are themselves useful components of these probe compositions containing said side chains.

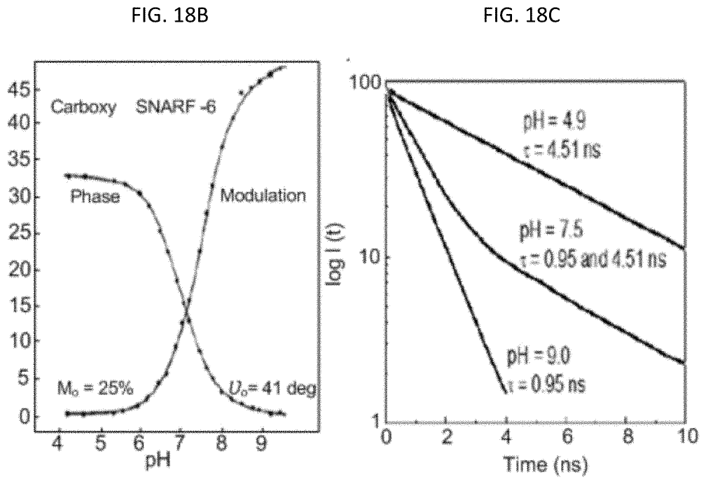

FIG. 18A is graph that illustrates an example carboxy SNARF-6 absorption spectra and effect of pH on the spectra, according to an embodiment. FIG. 18B and FIG. 18C are graphs that illustrate example time dependent decays recovered from full frequency-domain measurements and analysis at a pH where the total steady state intensities are close to equal, according to an embodiment. FIG. 18A through FIG. 18C simply demonstrate that a SNARF family of probe, known commercially as carboxy-SNARF-6, is a useful component of a probe composition embodiment. As can be seen in FIG. 18A, the absorption spectral peak amplitude or wavelength varies widely with varying levels of pH (traces shown for pH values of 9.5, 8.4, 7.9, 7.4 and 6.2, respectively). For this reason, probe compositions synthesized with this component are useful for determining pH using excitation-ratiometric or emission-ratiometric measurements.

FIG. 18B shows a there is a pH-dependent change in average lifetime, which was recovered from full frequency-domain using a single modulation frequency. This change in phase angle shows that the mean decay time changes from 4.51 nanoseconds (ns) at low pH to 0.95 ns at high pH.

For this reason, these differences are exploited for use in correlating these changes to a given pH level in tears. The addition of a hydrophobic carbon side chain to these probes results in a probe composition 150 embodiment useful for Si-HG contact lenses (SiHG-CL).

### Example 11: Alternate pH Detecting Probe Compositions

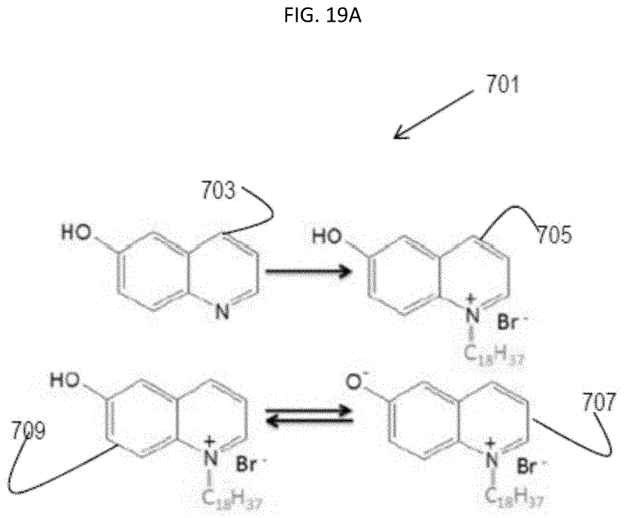

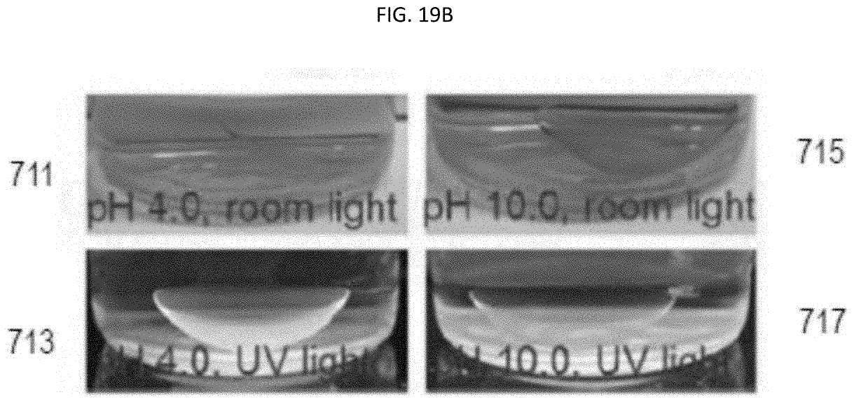

In addition to modified SNARF probe compositions mentioned above, quinolinium based (e.g., hydroxyquinoline) probe compositions may be used for detecting pH. FIG. 19A is a chemical diagram that illustrates an example synthesis scheme for a probe composition for useful to measure and detect pH in a contact lens, according to an embodiment. FIG. 19B is a set of images that illustrates how an example Quin C-18 probe fluoresces at various pH levels when exposed to UV light, according to an embodiment. It was discovered that a 8-18 alkyl or allyl side chain is useful for binding the probe composition in the particular silicone interstices of the SiHG contact lens. In the embodiment of FIGS. 19A and 19B, an 18-carbon side chain was used.

FIG. 19A shows a synthesis scheme 701 for embodiments of probe compositions used to measure and detect pH (concentration of hydrogen ion, H⁺) in a contact lens. Hydroxyquinoline 703 was reacted with 1-bromo-octadecane to form probe composition 150 forms 705, 707, 709. As used herein, 705, 707, 709 are collectively referred to as “6OH—N-C18H37-QBr” or HQ-C18.

Structures 705, 707, and 709 differ based on the level of ionization of the oxygen in the hydroxyl group. Depending on the pH, the hydroxyl group will be in the form OH⁻ (705), O⁻ (707), or OH (709). FIG. 19B shows how the probe 150 fluoresces differently at various pH levels when exposed to UV light. Image 711 indicates a clear lens in ambient light at a pH of 4.0. Image 715 indicates a yellow tint in ambient light at a pH of 10.0. Image 717 indicates a yellow glow in UV light at a pH of 10.0. Image 713 indicates a blue glow in UV light at a pH of 4.0.

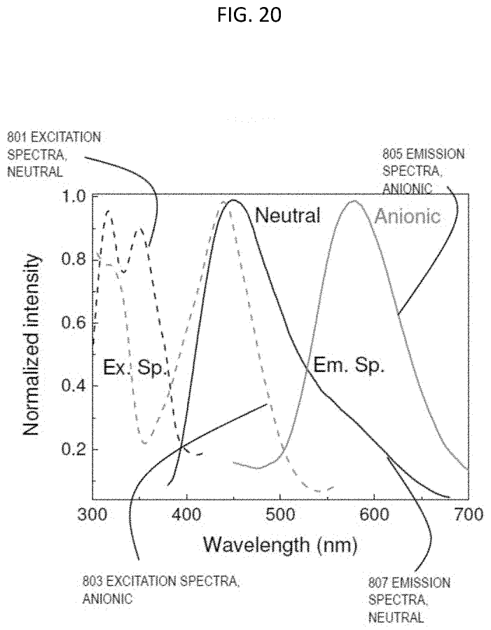

The 6OH—N-C18H37-QBr probe composition has been shown to be useful for detecting pH changes in a SiHG-CL. This probe composition 150 displays change in both the absorption and emission spectra. Because both emission and absorption spectra change, the pH can be measure using either the excitation or emission intensity ratios. FIG. 20 is a graph that illustrates an example excitation spectra (left) and emission spectra (right) of 6-OH—N-C18H37-QBr in Biofinity™ contact lens in water, according to an embodiment. Because of the differences in the spectra at various pH levels, the probe composition 150 is suitable for the detection of pH in a SiHG-CL because it not only responds to changes in pH, but due to its hydrophobic side chain, it will not wash out of the contact lens. The probe composition bound strongly to a SiHG-CL and could not be washed out by repeated rinsing.

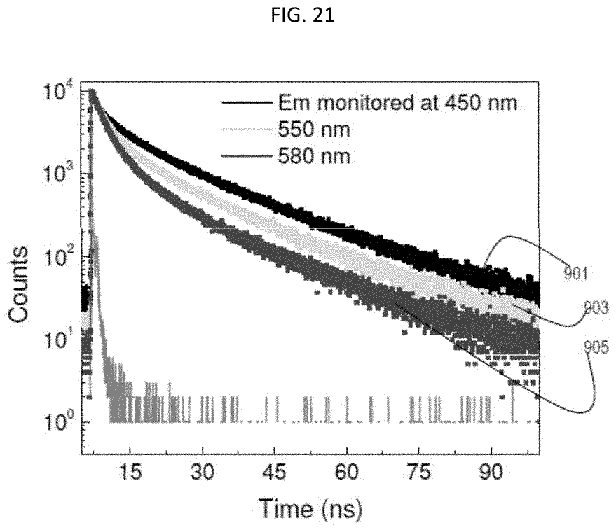

Not only does the embodiment probe composition, 6OH—N-C18H37-QBr, display changes in spectral shifts at varying pH levels, but there is also a significant effect of pH on lifetime of the fluorescence intensity. FIG. 21 is a graph that illustrates example intensity decay of 6OH—N-C18H37-QBr in a Biofinity™ contact lens in water, according to an embodiment. Point clusters 901, 903, and 905 represent the emission monitored at 450 nm, 550 nm, and 580 nm, respectively.

An alternate structure which may be used as a pH probe is 6OH—N-Allyl-QBr or HQ-C3, below:

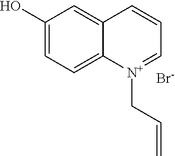

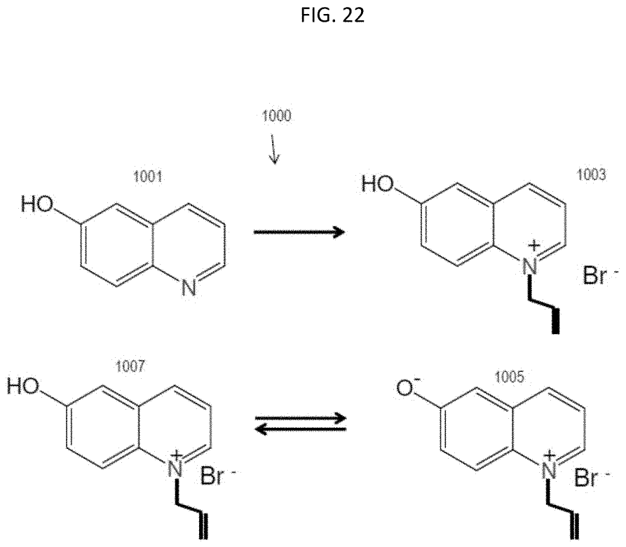

FIG. 22 is a chemical diagram that illustrates an example synthesis scheme for 6-OH—N-Allyl-QBr, according to an embodiment. This synthesis scheme is similar to the one shown in FIG. 19. Hydroxyquinoline 1001 is reacted with a three-carbon hydrocarbon to form three forms of 6-OH—N-Allyl-QBr, existing in equilibrium with one another. These are represented as structures 1003, 1005, and 1007, and are collectively known herein as 6-OH—N-Allyl-QBr. As with the 6OH—N-C18H37-QBr, the 6-OH—N-Allyl-QBr probe composition has been shown to be useful for detecting pH changes in a SiHG-CL. This probe composition displays change in both the absorption and emission spectra. Because both emission and absorption spectra change, the pH can be measure using either the excitation or emission intensity ratios.

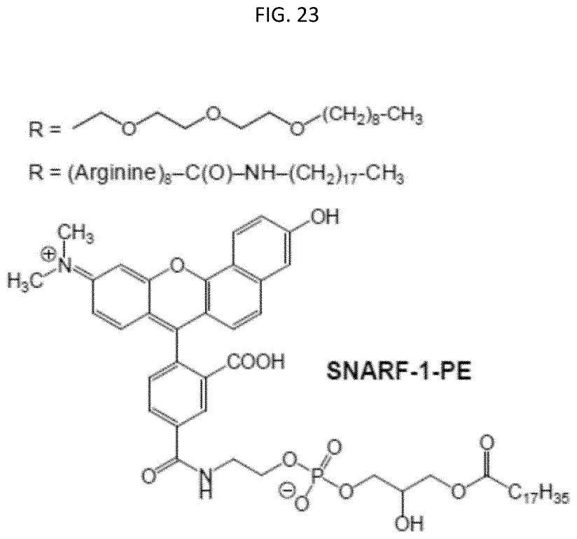

FIG. 23 is a chemical diagram that illustrates example hydrophobic side chains which may be incorporated into a certain pH-detecting probe compositions disclosed, as well as an example pH probe having a hydrophobic side chain, such that the hydrophobic portion binds the probe composition at an interface in SiHG lens, according to an embodiment. The R groups of FIG. 11 are hydrophobic side chains which may be incorporated into a certain pH-detecting probe compositions disclosed, such that the hydrophobic portion binds the probe composition at an interface in SiHG-CL. In this embodiment, the side chains contain separating units of polyethylene glycol or arginine peptide.

The main molecule shown in FIG. 23 is a probe composition consisting of SNARF-1 bound to lyso-phosphosphatidylcholine (lyso-PE). This embodiment of a probe composition is referred to as SNARF-1-PE herein. This structure is formed by coupling a commercially available active ester of SNARF-1 with lyso-PE. Advantages of this embodiment are that it may be more easily and spontaneously bound to the SiHG-CL. Lyso-PE is charged and so the probe composition therefore is unlikely to fully enter the silicone regions of the lenses. This is useful because the probe compositions advantageously straddle the channels such that the analyte binding portions are exposed to the aqueous environment. If the probe composition were wholly soluble in the silicone portions of the SiHG-CL, then it would not be available to bind (and therefore detect) various analytes. Another advantage of the SNARF-1PE probe composition is that the lyso-PE micelles are approximately 4 nm in diameter, which is small enough to enter the nanochannels of various SiHG-CL (which are about 50 nm in width).

### Example 12: Selection and Synthesis of Two Sodium-Sensitive Fluorophores

The epithelial cells in cornea are closely packed with tight junctions and generally not permeable to ions without active transport. The epithelial cells actively transport Na⁺ from the tear film into the stroma. It is likely that any damage to the epithelial layer will result in Na⁺ concentration differences between the PLTF and PolTF. Many sodium-sensitive fluorophores (SSF) are known, but in preferred embodiments, the practitioner has the ability to distinguish the two SSF from the spectral data and the fluorophores have sodium affinity constants in the physiological range for tears. Almost all azacrown ethers which are attached to the fluorophores by the fluorophores for the PLTF and one or two azacrown nitrogen atoms.

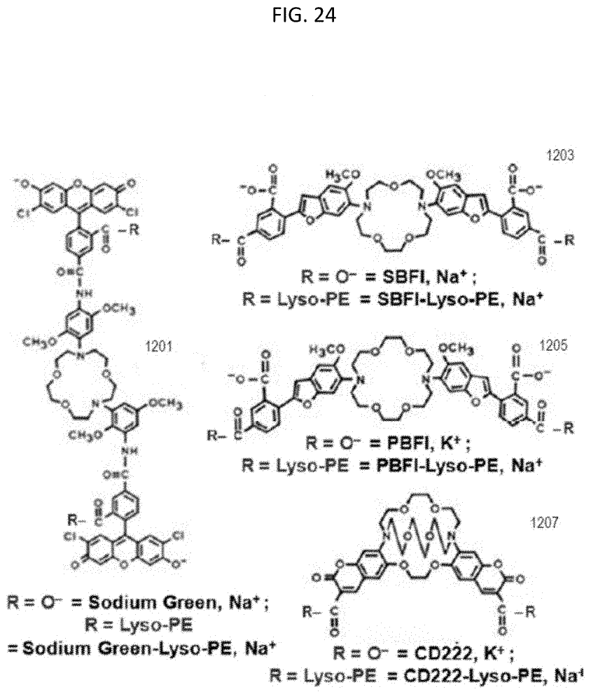

The first reported SSF useful in cells was SBFI (sodium binding benzofuran isophthalate). SBFI requires UV excitation at wavelengths from 320 to 400 nm. These short wavelengths could result in high autofluorescence from the eyes and could result in UV photodamage. Therefore, SBFI is not suitable for use in the invention. CoroNa™ Green and CoroNa™ Red are preferred SSF (see FIG. 24). Both of these probes can be excited with visible wavelengths near 450 nm. The emission spectra are widely separated with peaks at 520 and 580 nm, and the probes can be selectively observed at these wavelengths. If there is spectral overlap, the multi-exponential decays due to spectral overlap can be resolved. An important property of these SSF are the binding constants which are reported to be 82 mM for CoroNa™ Green and 200 mM for CoroNa™ Red. These binding constants are ideal for sodium sensing in tears. The probes in FIG. 24 cannot be used directly but are covalently linked to polylysine (SG-PL) for polylysine for contact lens surface localization. From starting with the water-soluble version of the probes, without the acetoxymethyl ester (AM) group which is used to make the probes permeable to cell membranes. If the non-AM probes are not available, the esters can be hydrolyzed to obtain free carboxyl groups for conjugation with polylysine.

One of the first and most widely used SSF for use with visible wavelengths is Sodium Green which contains two fluorescein-like groups bound to an azacrown ether are similar to non-toxic fluorescein except for the two additional non-reactive chlorine atoms. SG was designed for measurements of Na concentrations inside cells, 4-20 mM, which would result in complete saturation of SG at the 120 mM Na concentration in tears. We reasoned that the strong Na⁺ affinity that may be due in part to the two free carboxyl groups, and conjugation of these groups with polylysine could decrease the Na⁺ affinity.

### Example 13: Example Na⁺- and K⁺-Detecting Probe Compositions

There is speculation that K⁺ has specific effects in DED because, in contrast to other electrolytes, K⁺ has an approximate 5-fold higher concentration in tears than in blood serum. The Na⁺ concentration can affect the severity of the DED condition. For this reason, probe composition embodiments can detect these analytes. As with earlier probe compositions, these embodiments are tailored for suitability in a contact lens or a similar material.

Fluorescent probes for Na⁺ and K⁺ are based on azacrown ethers attached to fluorophores using the nitrogen atoms. FIG. 24 illustrates example chemical structures of an Na⁺ probe and ultraviolet (UV) analogues for use with UV light (SBFI), according to various embodiments. Structure 1201 is a visible wavelength Na⁺ probe composition based on a Sodium Green base, but where the R groups may be lyso-PE. As with the above, the purpose of the lyso-PE would be to keep the probe composition partially exposed to the aqueous environment, and not wholly submerged within the hydrophobic portions of the contact lens. Structures 1203 and 1205 are yet other embodiments of probe compositions and comprise a sodium-binding benzofuran isophthalate (SBFI) molecule (for detecting Na⁺) and a potassium-binding benzofuran isophthalate (PBFI)(for detecting K⁺), respectively, modified with lyso-PE at the respective R groups. Another embodiment of a probe composition for detecting K⁺ consists of the commercially available coumarin-based probe known as CD222 modified with a lyso-PE side chains in the “R” position (structure 1207) as shown in FIG. 24.

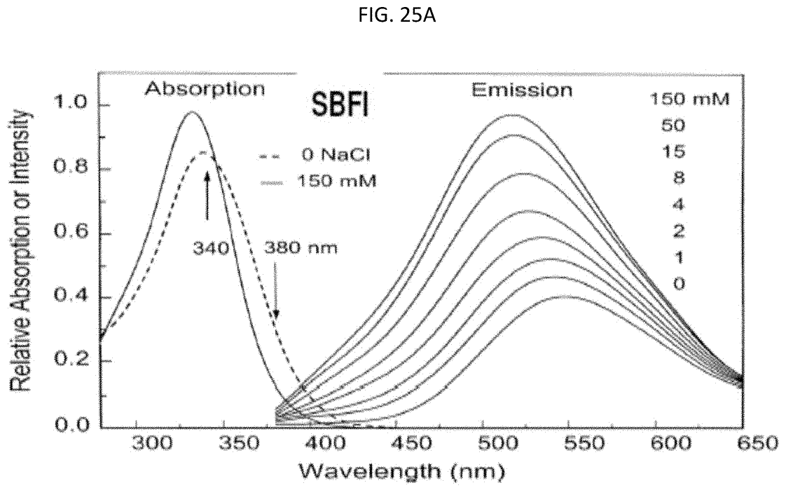

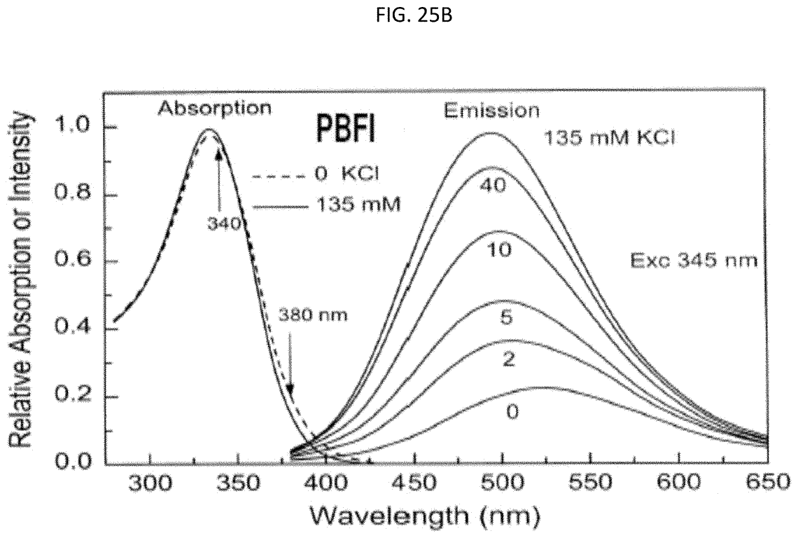

Correspondingly, FIG. 25A and FIG. 25B show that the emission spectra of SFBI and PBFI, respectively. FIG. 25A is a graph that illustrates example Na⁺ and K⁺ dependent emission spectra of SBFI, according to various embodiments. FIG. 25B is a graph that illustrates example Na⁺ and K⁺ dependent emission spectra of PBFI, according to an embodiment. As can be seen, in the figure, the emission spectra vary based on varying levels of concentration, which is indicated in the graph in various (mM) levels. As such, these probe compositions would be useful for correlating spectra with concentration levels.

### Example 14: Sodium Probe Binding

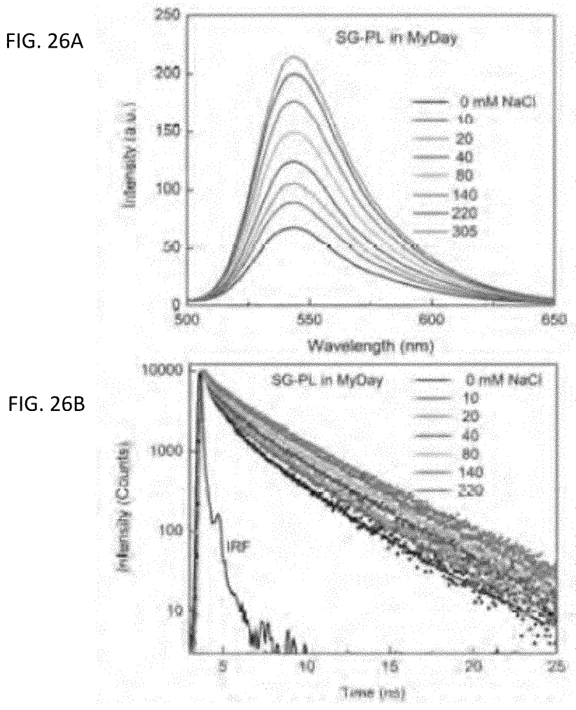

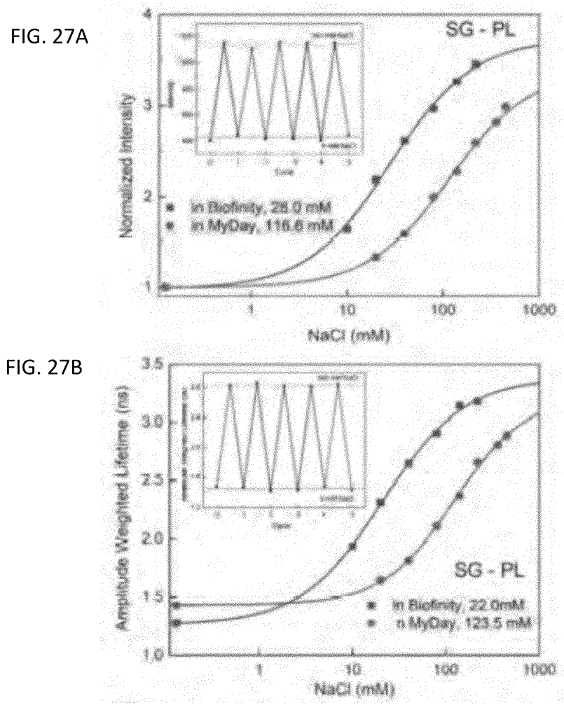

Biofinity™ and MyDay™ contact lenses were used for initial work to test whether SG-PL would bind to the MyDay™ lenses with a much lower silicone content. In both lenses, SG-PL displayed an appropriate 3-fold change in fluorescein intensity and lifetime (see FIG. 26), and the Na⁺ affinity shifted to bring SG-PL close to the tear sodium concentration (see FIG. 27), but it would be advantageous to have a Na⁺ affinity be about 2-fold lower for greater sensitivity changes in Na⁺ concentration. The methoxy groups on the phenyl rings contribute to the strong Na⁺ affinity of SG, and removal of the methoxy closest to the azacrown ether can decrease Na⁺ affinity as seen for other azacrown structures. Both the intensity and lifetime changes were completely reversible (see FIG. 27, inserts). Tears contain a large number of different proteins, the most dominant being lysozyme and human serum albumin (HSA). The sodium responses were not affected by these proteins.

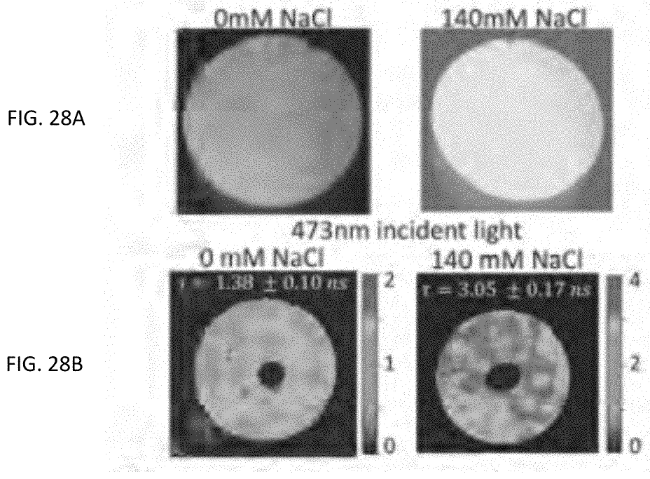

Intensity and FLIM images of Su-PL are shown in FIG. 28. The labeled contact lenses can provide images of the entire lens to detect localized changes in Na⁺ concentrations. The top panels show the uniform labeling with SG-PL. The intensity is increased about 3-fold in the presence of Na⁺ but are difficult to quantify in a photograph with auto-scaling. The lower panels are lifetime images which show a change from 1.38 ns to 3.05 ns in the presence of sodium.

### Example 15: Example Mg²⁺- and Ca²⁺-Detecting Probe Compositions

It is not yet known for certain the role of Mg²⁺ and Ca²⁺ in DED because means for measuring these ions has not been previously available. However, it has been suggested increased Ca²⁺ concentrations could indicate some form of ocular defect because calcium levels in tears is typically 5-fold lower in tears than blood. Thus, a method for detecting Mg²⁺ and Ca²⁺ ions in tears can be useful for research purpose and potentially for diagnostic purposes as well. Embodiments of probe compositions have been developed that are useful for detecting concentrations of these ions. As with the previous embodiments, the probe compositions are developed so that they traverse the silicone-hydrogel interface in a SiHG-CL with a hydrophobic portion anchored in the silicone interstices and the analyte binding portion in the hydrogel nanochannels to detect analytes in the aqueous flow.

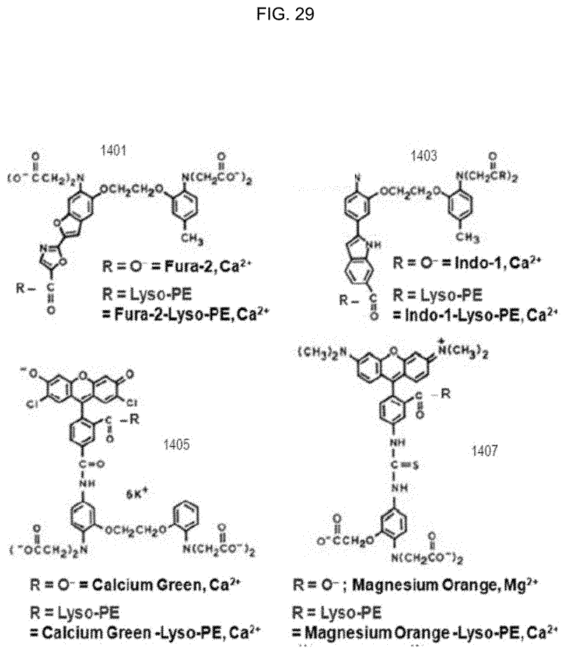

FIG. 29 is a set of chemical diagrams that illustrate example structures of calcium and magnesium ion-detecting probe compositions and proposed lyso-PE derivatives for binding the probe composition into a contact lens, according to various embodiments. FIG. 29 shows four structures 1401, 1403, 1405, and 1407 for detecting these ions. Structures 1401, 1403, and 1405 detect calcium levels and structure 1407 detects Mg²⁺. As with the probe composition embodiments above, lyso-PE is added to various probes in the location corresponding to the R group in FIG. 29. It should be noted that structure 1401 may also be modified by adding an 8-18 alkyl side chain in place of the lyso-PE.

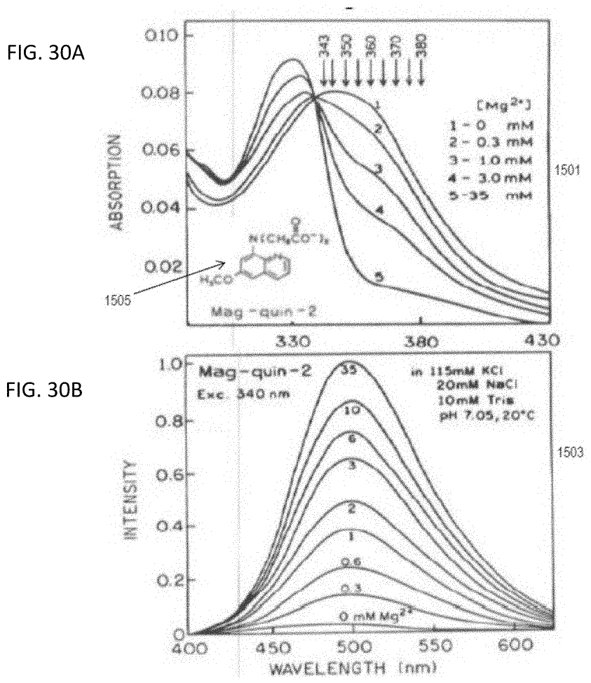

FIG. 30A and FIG. 30B are graphs that illustrate example absorption and emission response, respectively, to magnesium as well as a structure for a magnesium-detecting probe composition, according to an embodiment. FIG. 30A includes an inset that shows another embodiment of a probe Mag-quin-2 1505 for detecting Mg²⁺. This probe can be modified by adding a hydrophobic side chain (such as an alkyl group having between 8-18 carbons) or lyso-PE group so that it is capable of binding at a silicone-water interface. The absorption spectra 1501 and emission spectra 1503 show that a modified version of Mag-quin-2 can be used as an excitation wavelength ratiometric probe for the same reason as other probes compositions disclosed. Another advantage is that the binding constant can be changed from 0.2 mM to 20 mM by changing the excitation wavelength from 340 to 365 nm. This effect is valuable in making a magnesium sensitive contact lens. If the binding at the silicone-hydrogel interface changes the magnesium affinity outside the physiological range, the apparent binding constant can be shifted to the most magnesium sensitive range by using LEDs with different wavelengths.

### Example 16: Example Cl⁻-Detecting Probe Compositions

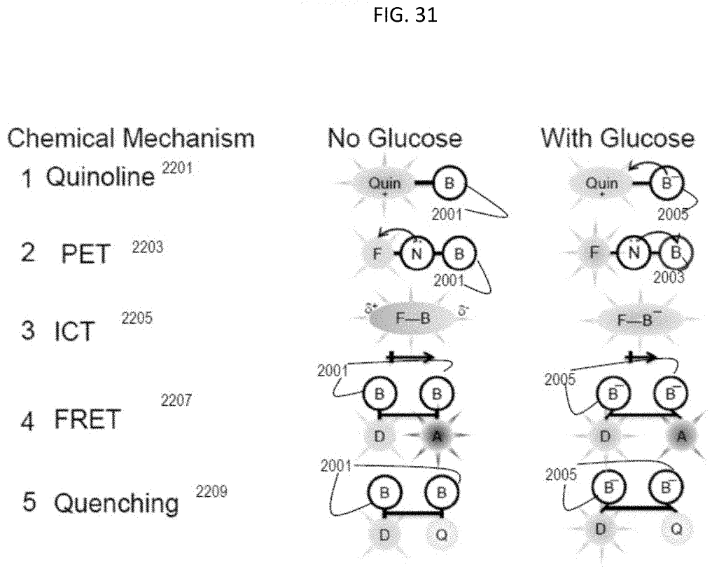

It is important to be able to determine chloride ion concentrations in tears as these concentrations are needed to calculate the total osmolarity of a patient's tears. Detection of the chloride ion by a typical probe occurs by the process of collisional quenching. Collisional quenching is discussed in reference to FIG. 31, but essentially means a probe in the excited state returns immediately to the ground state without emitting a photon, upon contact with chloride ion due to diffusion. The probe is not destroyed by the quenching process and remains available for further excitation.

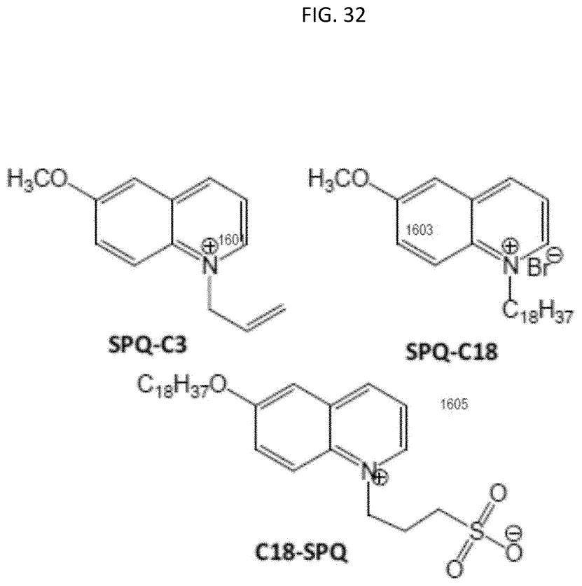

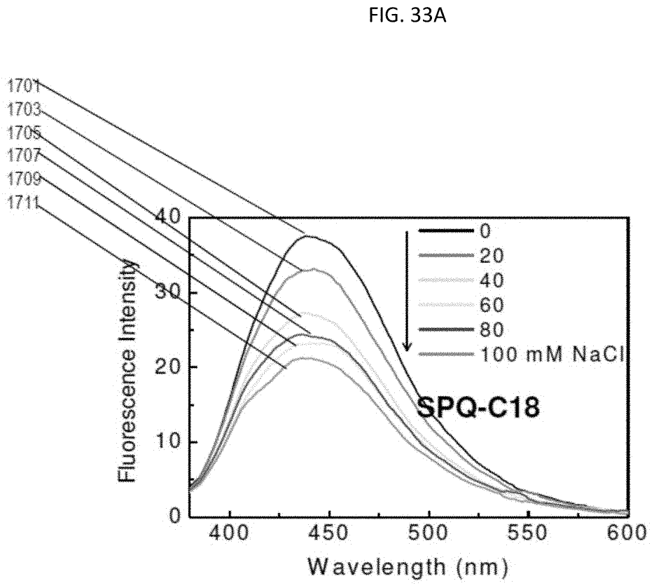

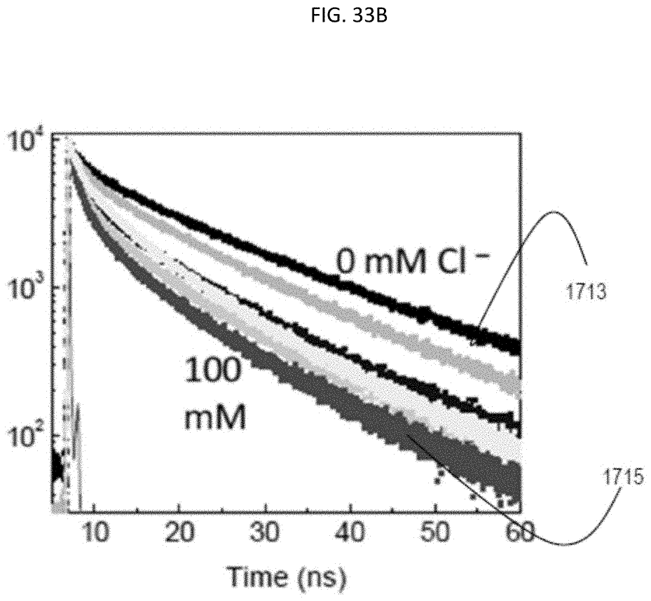

Previously known reported chloride probes would not work as components in probe compositions for use in a SiHG-CL. FIGS. 32 and 33 demonstrate various chloride probes and their various properties. FIG. 32 illustrates example probe composition structures for detecting chloride ion, according to an embodiment. In FIG. 32, probe SPQ-3 is too sensitive to chloride ions because it is almost completely quenched at concentrations that are lower than the typical eye concentration of chloride ion. This could be a problem because it would not be possible to get an accurate read of actual chloride ion concentrations in tears in this situation. To develop a chloride ion probe that would be less sensitive to quenching, it was recognized that the addition of a carbon side chain (preferably 8-18 carbons in length), as in probe composition embodiment 1603 (SPQ-C18) would reduce the quenching sensitivity of the probe to chloride ions.

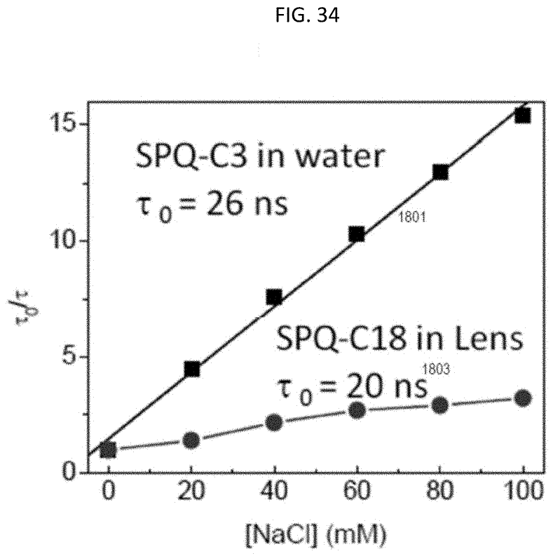

When tested, SPQ-C18 1603 exhibited a 7-fold reduction in quenching sensitivity when bound to a SiHG-CL. FIG. 33 illustrates an example emission spectra for SPQ-18 in an Stenfilcon (Aspire™) contact lens, according to an embodiment. As shown in FIG. 33, the fluorescence intensity at various concentrations of chloride ion (from NaCl salt ranging from 0-100 mM) ranged from around 20-40% at 450 nm (see traces 1711 and 1701, respectively). In contrast, the fluorescence intensity SPQ-C3 ranged from around 20-100% at 450 nm for concentrations ranging from 0 to 100 mM (not shown). This is a problem because the normal chloride ion concentration in tears is about 118-138 mM. Therefore, unlike SPQ-C31601, SPQ-C18 1603 is only about 50% quenched at typical chloride ion concentration in tears, leaving remaining ability to detect if there are abnormally elevated chloride ion concentrations in an eye. This reduction in quenching renders the disclosed probe composition suitable for its intended purpose and is also demonstrated by the comparison of lifetime Stern-Volmer plots for SPQ-C3 v. SPQ-C18 in FIG. 34. FIG. 34 is a graph that illustrates an example comparison of lifetime Stern-Volmer traces for SPQ-C3 in water and SPQ-C18 in a Stenfilcon (Aspire™) contact lens, according to an embodiment.

These measurements were taken with the probes in a Stenfilcon A (Aspire™) contact lens, a typical SiHG-CL. FIG. 34 illustrates an example time-dependent decay of SPQ-18 (1603) in the presence of the chloride ion, according to an embodiment. FIG. 34 is included to show that this spectral property of this probe composition embodiment 1603 is also useful for correlation with various concentrations of chloride ions. For instance, 0 mM chloride ion (trace 1713) is distinct from trace 1715 which was taken at 100 mM concentration.

In addition, probe composition embodiment C18-SPQ 1605 has an additional water soluble sulfonic acid group, which allows for more chloride ion sensitivity, since this portion of the probe composition would orient in the aqueous nanochannels where chloride ion would be present.

Recent developments in silicone hydrogel contact lens (SiHG-CL) and fluorescent probes also provide an opportunity to perform measurements of biomarker concentrations with specially labeled (probe composition-infused) contact lenses for detection of keratitis. Keratitis may originates with chemical changes occurring in the tear layer surface between the inner surface of the contact lens and the outer layers of the cornea. There are no standard methods to perform measurements in this region which both has a volume much smaller than the tear volume of an eye and is inaccessible to any type of sample collection without disturbing the sample itself.

The water channels in the nanoporous polymer networks (NPN) of the hydrogel are probably too small for penetration of large proteins such as lysozymes, bacteroferrin and IgA. However, the water channels may be large enough to admit smaller molecules such as defensins, which are typically small peptides with 29 to 42 amino acids and are constrained into folded forms by six conserved cysteine residues. Human beta-defensin-2 [HBD-2] is produced rapidly following stimulation of epithelial cells and increased concentrations of HBD-2 is often the earliest sign of infections, even before the infection is visible to an ophthalmologist. The strong resistance of corneal epithelial to infection is most likely due to HBD-2 which is rapidly produced in response to some bacteria (e.g., P. aeruginosa) and specific lipopolysaccharides. Defensins could be detected by aptamers or peptide aptamers which are much smaller than antibodies and could be incorporated into a SiHG-CL, either in the NPN or on the lens surface.

### Example 17: Linkers for Ion-Sensitive Fluorophores

Binding of ISF to the lenses also can be accomplished using proteins. Both hen egg white lysozymes (HWL) and human serum albumin (HSA) bind to SiHG lenses. In many lenses, the binding is localized to the surface. The extent of surface localization depends on the particular contact lens polymer. Human egg white lysozyme and HAS, which have covalently bound ISF, are suitable for use. Since these same proteins are with all lenses the ion responses may be similar in different contact lens polymers. Any protein or peptide that is non-toxic in use as part of a contact lens can be used.

### Example 18: Example Contact Lens Glucose Detection Embodiments

Recent developments in silicone hydrogel contact lens (SiHG-CL) and fluorescent probes provide an opportunity to perform measurements of glucose concentrations with specially labeled (probe composition-infused) contact lenses for detection of diabetic hyperglycemia for disease control and diabetic hypoglycemia due to overdose of insulin.

As mentioned, Glu-SFs are based on molecules containing boronic acid in which boronic acids binds reversibly to glucose. FIG. 35 is a chemical diagram that illustrates an example generic molecule containing diboronic acid moieties in the sugar-bound and sugar-unbound conformations, according to an embodiment. FIG. 35 shows a generic molecule containing two boronic acid moieties 1901. FIG. 35 is included to show the boronic acid conformation in a sugar-bound state 1903, 1905, 1909 versus a sugar-unbound state 1901. (The term “sugar” in this figure may represent a molecule of glucose).

This generic composition is the probe composition 150 in some embodiments, in which case, the R group 1905 is a combination of the fluorophore portion 154a and/or 154b, together with the spacer portion 156 (if appropriate), the hydrophobic portion 151, and the hydrophilic portion 152. In that case, the boronic acid moieties 1901, 1903 would be the analyte binding portion 153.

In this example, the analyte to be bound is glucose. As is shown in FIG. 35, boronic acid moiety 1901 is in the trigonal (sugar-unbound) form. Boronic acid moiety 1903 is in the tetrahedral conformation when in the sugar-bound form. These structures can change confirmation between structure 1907 (the sugar bound to both boronic acid moieties, or structure 1909 with the addition of another glucose, two molecules of glucose can be bound—one for each boronic acid.

Glucose binding changes the electron donating-accepting (Lewis base-acid) ability of boronic acid which in turn affects the adjacent fluorophore portion 1905. This binding of glucose to the boronic acid moieties of any of the embodiments disclosed herein alters the fluorescence spectra, intensity or lifetimes of nearby fluorophores by different mechanisms. These fluorescence features can be correlated to the glucose levels in a sample of interest.

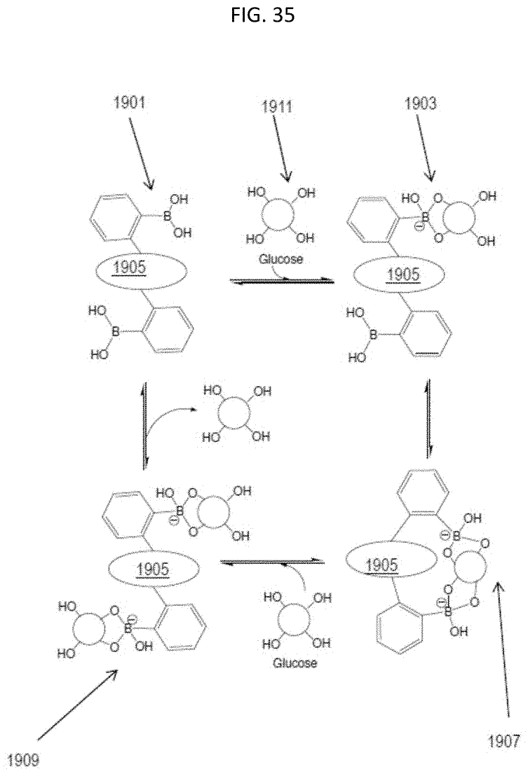

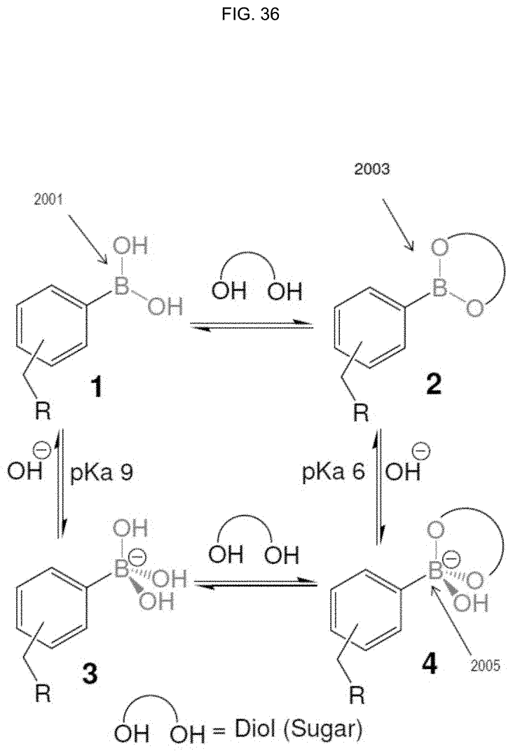

Glu-SFs typically have one or more boronic acid moiety which binds to glucose (although one is shown in FIG. 36 for illustrative purposes, probe compositions have been prepared with more than one boronic acid moiety). When binding occurs, the trigonal geometry of the boron changes to a tetrahedral geometry with more electron density on the boronic acid moiety.

This change in geometry and/or electron density can affect the spectra, intensity or lifetimes of nearby fluorophores by different mechanisms, which are summarized in FIG. 31. FIG. 36 shows a boronic acid structure, but embodiments of probe compositions 150 herein employ a diboronic acid. Depending on the fluorophore portion used in the probe composition, one or more of these properties (spectra, intensity or lifetimes) is affected. For example, collisional quenching processes may result in changes in intensity without significant effects to the emission spectrum. Intensity measurements are useful and simple for relating a concentration of an analyte to the intensity of fluorescence, however, generally speaking, shifts in wavelength of spectral peak are preferable to measuring intensity. While intensity measurements are simple and straightforward, they may be inadequate at times in real world situations due to for example, the turbidly of a sample, non-optical surfaces may be misaligned, dependence on measurement geometry, and generally, intensity-based sensing is not particularly sensitive to small changes in intensity. Finally, lifetime-based sensing measures changes in lifetime of fluorophores in response to analytes, and is advantageous in that fewer probes display spectral shifts, but a wide variety of quenchers and/or molecular interactions result in changes in the lifetimes of fluorophores.

One of the objectives was to design probe compositions which can be used to detect various analyte concentrations, but using these various types of properties and through different mechanisms of electron density affects.

FIG. 31 illustrates various example mechanisms by which changes in geometry of a boronic acid moiety can affect the spectra, intensity, or lifetimes of nearby fluorophores. It shows five different mechanisms by which the various probe compositions disclosed display their given spectral properties to be detected (thereby correlating to a given analyte concentration). These photophysical mechanisms include quenching (e.g., FRET or collisional quenching), photo-induced electron transfer (PET), and intramolecular charge transfer (ICT) which can result in changes in lifetime and spectral shifts due to the different electronic distributions when bound to glucose. In the SiHG-CL, these photophysical processes will occur at the water-silicone interface within the SiHG-CL, and require different amounts of molecular motion.

The various encircled B symbols in FIG. 31 are the various forms of the boronic acid shown in FIG. 36. Also, FIG. 36 shows a boronic acid structure for the purpose of showing the binding of boronic acids to sugars and to show different forms of the various boronic acid moieties can take. However, embodiments of probe compositions 150 herein employ a diboronic acid, for example a diboronic acid shown in FIG. 35. Therefore, reference to FIG. 36 and FIG. 31 together is appropriate. Below is a summary of the various mechanisms through which probe compositions 150 described herein operate.

Turning to the first mechanism represented in FIG. 36 and FIG. 31, quinoline mechanism 2201 occurs with certain quinolinium based probe compositions and simply refers to the electron donor-acceptor interactions that take place in quinolinium based probe compositions disclosed when glucose binds to the boronic acid moiety. The boronic acid in this type of probe composition is in the form 2001 in the unbound form. When bound to glucose, the boronic acid moiety acquires the additional hydroxyl group and takes the form 2005. The electron movement in this mechanism proceeds by a shift in charge density from the bound form of boronic acid moiety 2005 moving to the quinolinium structure. Note that FIG. 31 refers to Mechanism 1 as “Quinoline,” however, this shall be understood to mean the mechanism for “quinolinium” based probe compositions disclosed below.

The second mechanism, shown in FIG. 36 and FIG. 31, 2203 is photoinduced electron transfer (PET). PET is an excited state electron transfer process by which excited electron is transferred from donor to acceptor. In the example 2203, the electron donor is the amino group, represented as the encircled “N.” Upon the binding of glucose, the diagram shows the electrons are taken from the nitrogen and that the boronic acid moiety takes the conformation 2003. Some Glu-SFs display intensity changes due to PET quenching. These Glu-SFs typically contain an aromatic fluorophore with a nearby amino group. Specific examples of probe compositions which work via this mechanism are given following this discussion on mechanisms generally. The amino group transfers an electron to the excited state fluorophore resulting in quenching because the exciplex is usually non-fluorescent. However, quenching is not a necessary consequence of PET. Electron transfer can result in formation of an excited state complex called an exciplex. An exciplex can display strong emission in non-polar solvents. Exciplex emission may occur in some SiHGs, but to a much lesser extent (or not at all) in HG lenses.

Still referring to FIG. 36 and FIG. 31, mechanism 3, intramolecular charge transfer (ICT) 2205 refers to a molecule having intramolecular electron transfer which occurs in a push-pull electronic structure of the excited state, resulting in positive and negative charge separation process in the molecule. ICT-based probe molecules typically include an electron donative group (hydroxyl group, amino group, etc.), and an electron withdrawing group (aldehyde group, benzothiazole, etc.) connected together. In the example shown, the “no Glucose” form of the probe composition has a longer dipole moment than the bound form. As provided in further detail below, a probe composition has been designed which will cause the overall charge distribution of the fluorophore portion to change upon the binding of glucose.

Mechanisms 2207 and 2209 in FIG. 31 refer to FRET and Collisional Quenching mechanisms, respectively. Both are quenching processes. “Quenching” refers to any process which decreases the fluorescence intensity of light emitting molecules. A variety of processes can result in quenching, such as excited state reactions, energy transfer, complex-formation and collisional or dynamic quenching. The chloride ion is a well-known quencher for quinine fluorescence.

Typical fluorescence microscopy techniques rely upon the absorption by a fluorophore of light at one wavelength (excitation), followed by the subsequent emission of secondary fluorescence at a longer wavelength. FRET involves a donor fluorophore in an excited electronic state, which may transfer its excitation energy to a nearby acceptor. One advantage of FRET mechanism 2207 that is it is independent of immediate environment of the Donor (D) and Acceptor (A) pair. Collisional quenching is similar to FRET and not dependent on the local chemical environment. In contrast to FRET, collisional quenching requires direct molecular contact between the fluorophore and quencher. Because of the short interaction distances, collisional quenching usually requires some diffusive motions to bring the molecules into contact.

FRET is due to a through-space interaction which occurs over distances from 1 to 10 nm. Molecular contact between the donor and acceptor is not necessary. FRET also depends on the absorption and emission spectra of the donor and acceptor. Mechanism 2209 shows that collisional quenching requires molecular contact of the probe with the quencher, at distances less than 0.5 nm. FRET requires an acceptor which absorbs at the donor emission wavelength. A quencher does not need to have absorption which overlaps the probe, and typically does not have such absorption.

### Example 19: Development of Polarity-Sensitive Probes

One issue in the development of the disclosed probe compositions is that they are designed to most frequently reside in the water-silicone boundary. If the probe composition were too soluble in water, it would wash out of the lens and not work for extended analyte monitoring. At the same time, the probe composition should not have so much nonpolar character that it was highly soluble in the silicone-based interstices of the contact lens material. Unless the probe composition resides at the water-silicone boundary, it would not both come into contact with the aqueous analytes and persist in place as the aqueous solution flows by.

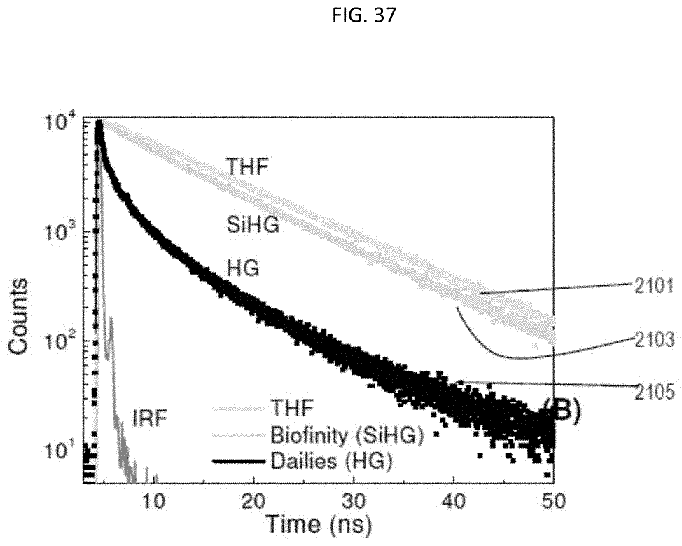

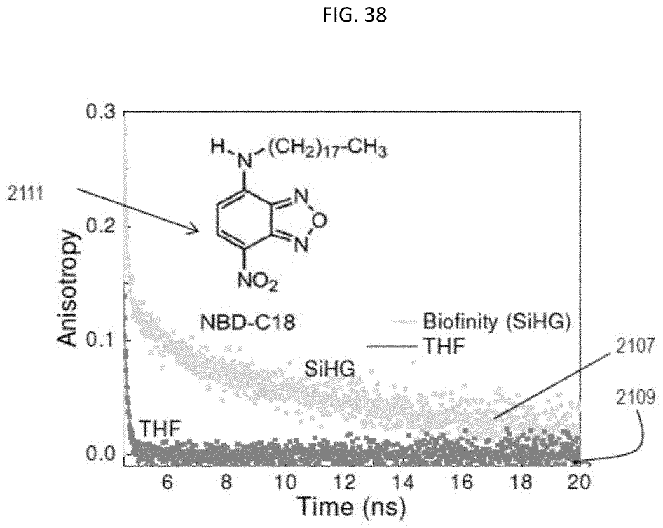

FIG. 37 and FIG. 38 are graphs that illustrate example intensity decays and anisotropy decays in THF and Biofinity™ lens, respectively, according to an embodiment. In this embodiment, Biofinity™ SiHG lenses are used with a small water-soluble probe nitrobenz-oxa-diazole (NBD) linked to a C18 chain 2111. It is known that if fluorophores bind to the silicone-rich regions this will be detected by their long correlation times. In the Biofinity™ SiHG (2103) lens, this probe displayed a single long decay time consistent with a non-polar environment (nonpolar environment shown using THF 2101). In the non-silicone HG lens, the emission intensities were very weak (trace 2105) and the decays were strongly non-exponential which is believed due to weak and/or heterogeneous binding to the HG lenses. The anisotropy decay in the SiHG lens shown in FIG. 38 showed a correlation time near 6 ns, which is comparable to those found for fluorophores in cell membranes.

Consistent with the above data, it was also confirmed that NBD-C18 2111 was completely bound and would not wash out. This was shown by the absence of signal outside the lens indicating no detectable fluorophore present in the surrounding solution. For this reason, a single C18 side chain is adequate for complete binding of a Glu-SF to a SiHG lens.

### Example 20: Quinolinium Glu-SFs

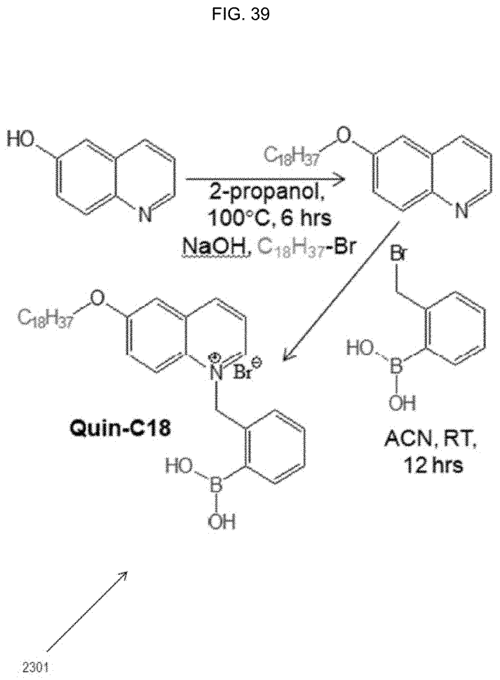

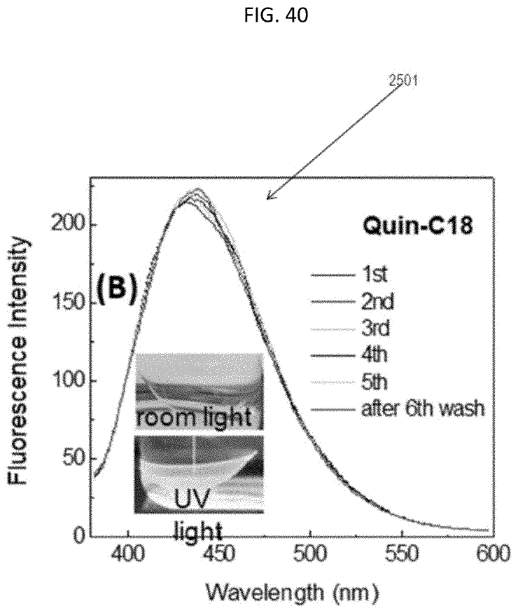

A standard quinolinium probe displays a useful spectra change in response to excitation with light, however it would not work for the intended purpose because it is too easily washed out of a contact lens. FIG. 39 illustrates an example reaction scheme for the preparation of probe composition Quin C-18 2301, according to an embodiment. Quin C-18 is hydroxyquinoline which has been modified to include a C18 alkyl side chain. In contrast to Quinoline alone, Quin-C18 could not be washed out of a SiHG lens even after repeated washings. FIG. 25 is a graph with an image inset that illustrates example persistence of fluorescent probe in SiHG contact lens after washing by showing the emission spectra of Quin-C-18 after repeated washing, and a photograph of the lens in room light and with UV incident light, according to an embodiment. The repeated washings are shown in FIG. 40, which shows that the fluorescence intensity remained relatively unchanged even after 6 washes. The blue emission from Quin-C18 in the lens can be easily seen in room light with a UV hand lamp as the cloudy lens in the lower insert image of FIG. 40. Binding of Quin-C18 also did not change the visual appearance of the lens.

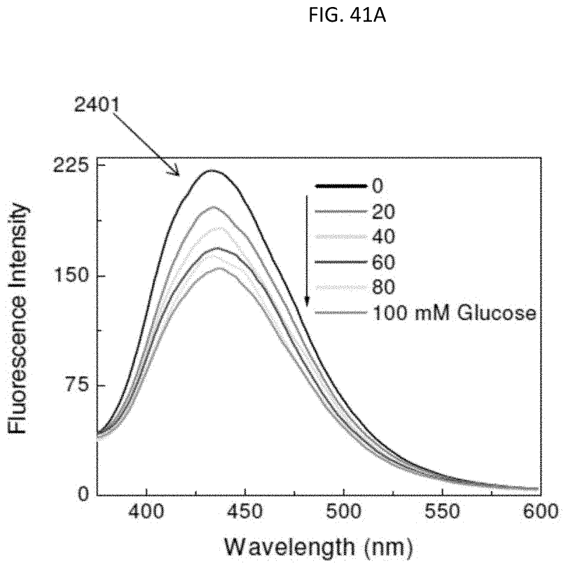

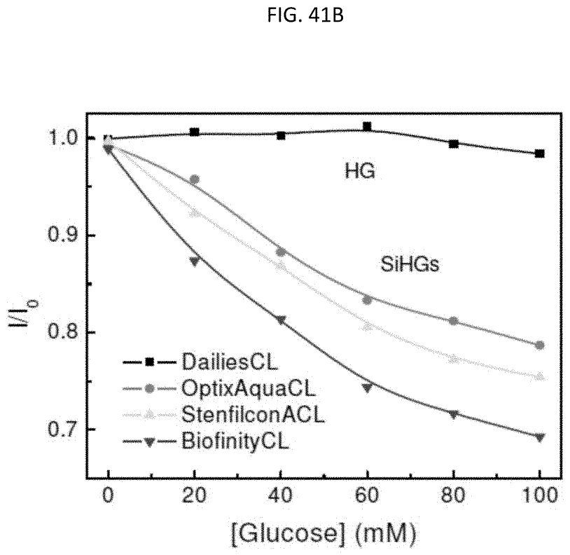

FIG. 41A is graph that illustrates an example glucose-dependent emission spectra of Quin-C18 within a Biofinity™ SiHG lens, according to an embodiment. FIG. 41B is a graph that illustrates example normalized intensities in a Dailies (HG) lens and in three SiHG, Stenfilcon A (Aspire™ 1 day) and Optix-Aqua™ lenses, according to an embodiment. The Quin-C18 was tested in four types of CL polymers, 3 SiHG lenses, and 1 standard HG. Addition of glucose resulted in a decreased intensity for Quin-C18 in the 3 SiHG lenses, but the weak emission intensity was unchanged in the standard HG lens (FIG. 41B). This result proves that Glu-SFs can be designed to bind to the water-silicone interface and remain functional to glucose.

### Example 21: Photo-Induced Electron Transfer Glu-SFs

A known probe for detecting and binding glucose is called ANDBA, shown below.

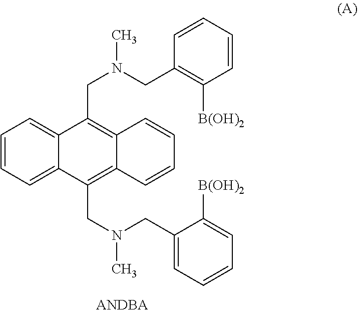 [SMILES: CN(Cc1ccccc1B(O)O)Cc1c2ccccc2c(CN(C)Cc2ccccc2B(O)O)c2ccccc12]

This probe has a strong response to glucose concentrations, but it was determined that these ANDBA probes did not respond to glucose in the HG or SiHG lenses.

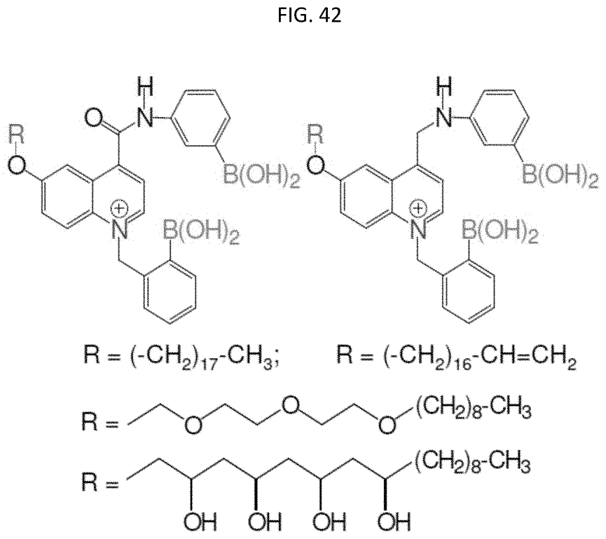

To remedy this, ANDBA-like probe compositions are synthesized. The differences include that these ANDBA-like probe compositions include alkyl side chains which tend to orient the diboronic acid moiety towards the aqueous phase. These probe composition embodiments are shown (FIG. 42). This figure is a chemical diagram that illustrates example diboronic acid Glu-SFs structures using a quinolinium nucleus for binding at an interfaces in SiHG lenses, according to an embodiment. These side chains contain spacer portions of polyethylene glycol or hydroxyl groups.

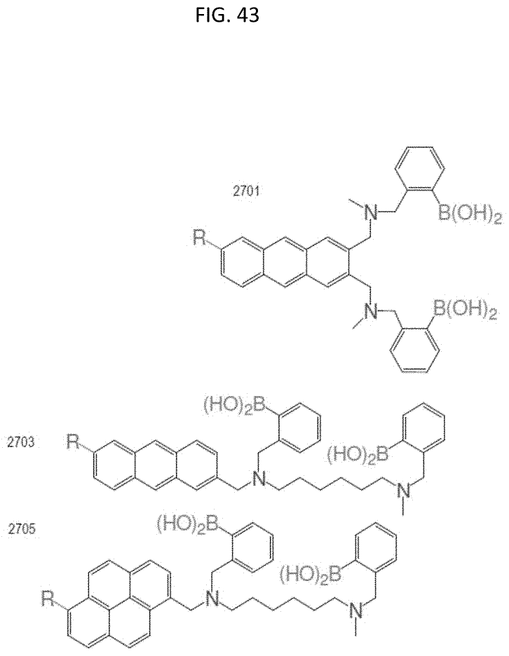

These embodiment probe compositions may also include further groups, such as polar or charged side chains. These side chains would also be added to ANDBA or the other PET probes of FIG. 42. Hydrophobic side chains with hydrophilic regions close to the anthracene are designed to expose the diboronic acid region more fully to the aqueous phase. The glucose response of ANDBA may have been restricted because the boronic acid groups are too far apart. FIG. 43 is a chemical diagram that illustrates example diboronic acid PET Glu-SFs for binding at an interfaces in SiHG lenses, in which the lower two structures are to displace the diBA more into the water phase, according to an embodiment. In this embodiment, the structure is modified so that the side chains are closer on the anthracene ring, depicted as structure 2701.

Alternatively, the phenyl rings could contain polar or charged side chains to keep this region in the water phase. Positively charged side chains are likely to increase the glucose affinity because of the negative charges on the boronated groups when bound to glucose. The affinity for glucose can also be increased by electron withdrawing groups on the phenyl rings.

Additional structures for PET base probe compositions 2703 and 2705 are shown in FIG. 43. These probe compositions contain a six carbon chain linker but the fluorophores are still close to nitrogen to allow PET to occur. The C6 linker is known to provide the optimal distance between the boronic acid groups for high glucose specificity and affinity.

ANDBA did not display exciplex emission, which prevents its use as a wavelength-ratiometric probe. If exciplex emission is needed, or if PET quenching does not occur in the SiHG, the structure can be altered to contain moieties which increase the tendency for PET. For example, the rates of electron transfer can be increased by the addition of halogens (Cl or Br) to the electron-accepting fluorophore or by using carbazole as an electron donor. Numerous alternative structures such as fluorenes can be used to develop Glu-SFs for use in SiHG lenses.

In contrast to the Quin-based probes, the response of PET probes probably requires more flexing to move the phenyl side chains towards a central fluorophore. The desirable amount of probe motion is probably larger for the structures with a C6 linker (2703, 2705) than for the structure 2701. This suggests the probe response depends on the microviscosity at the water-silicone interface. If this interface is too viscous for the 7 ns decay time of anthracene, then a pyrene nucleus can be used which has a decay time near 200 ns and thus provides more time for the exciplex to form.

FIG. 43 shows diboronic acid PET Glu-SFs for binding at interfaces in SiHG lenses. The lower two structures are introduced to displace the diBA more into the water phase. R is a long alkyl chain which may be the R chains shown in FIG. 42 or similar.

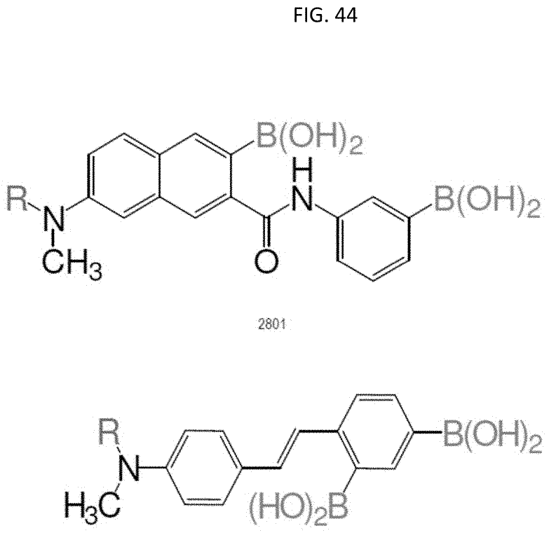

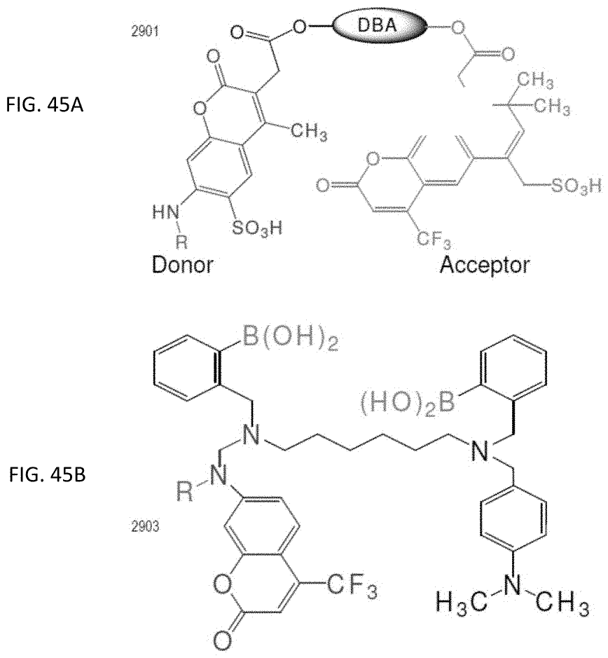

FIG. 44 is a set of chemical diagrams that illustrate diboronic acid ICT Glu-SFs for binding at interfaces in SiHG lenses, according to an embodiment. R is a long alkyl chain which may be the R chains shown in FIG. 42 or similar. FIG. 45A shows Glu-SF structures using a diboronic acid on a C6 linker with FRET mechanism. R is a long alkyl chain which may be the R chains shown in FIG. 26 or similar.

### Example 22: Probe Composition Embodiments

A number of Glu-SFs undergo intramolecular charge transfer (ICT) upon excitation. The extent of charge transfer depended upon glucose binding to the boronic acid, which changes its electron affinity. These probes were designed based on the known properties of diphenylpolyenes and the electronegativity of groups at each end of the molecule. These probes displayed large spectral peak wavelength shifts when bound to glucose in buffers. However, no such spectral shift was observed when the ICT probes were put into a conventional HG (not shown). For use in an SiHG-CL, these compounds are synthesized to include a hydrophobic side chain for use in an SiHG-CL. FIG. 44 shows diboronic acid ICT Glu-SFs for binding at interfaces in SiHG lenses which may be synthesized for this use. Glucose-dependent spectral shifts will occur because the overall charge of the probe changes upon binding glucose, so its position relative to the interface will change. If the emission spectrum is comparable to the spectrum in water, a more hydrophobic moiety on the nitrogen is added in some embodiments to shift its location towards the interface. Molecular flexing of the ICT probes is not needed for a glucose response. The immediate environment around the probe reorients in response to the new charge distribution of the probe.

### Example 23: Quenching Probe Composition Embodiments (FRET and Collisional Quenching Mechanisms)

FIG. 45 is a set of chemical diagrams that illustrate Glu-SF structures using a diboronic acid on a C6 linker with FRET mechanism, according to an embodiment and a Glu-SF structures using a diboronic acid on a C6 linker with a collisional quenching mechanism, according to an embodiment. FRET based Glu-SF is shown in FIG. 45 as structure 2901. The linkers may be six carbons long which is known to be optimal length for glucose selectivity and changes in FRET upon glucose binding have been demonstrated for this linker. The use of an aliphatic linker instead of an aromatic linker may also be employed and will allow the D-A distance to be modified for use in various SiHGs.

In some embodiments, coumarins are used as donors and acceptors because they have two aromatic rings and contain various polar groups, which keeps the probes in the water-rich regions of the SiHGs. A large number of coumarins are available covering a wide range of wavelengths. At least two types of FRET probe compositions are suitable for this purpose in various embodiments. In one embodiment a Glu-SF with a fluorescent acceptor is used as a wavelength ratiometric probe with emission near 460 and 560 nm. In some embodiments, a Glu-SF with a non-fluorescent acceptor is used as a lifetime-based sensor using the donor emission (not shown). The donor lifetimes are a more reliable measurement of FRET than the intensity ratios. FRET is sensitive to background emission and other potential artifacts such as donors without an acceptor; or incomplete separation of the D and A emission spectra. The donor decay time measurement has the advantage of providing the most reliable measurement of the FRET efficiency.

For the FRET based Glu-SF, the donor and acceptor (D and A) are in close proximity so D-A pairs with small Ro values are an advantage. D-A pairs with Ro values, ranging from 6 to 33, using only a single donor are suitable. The Rₒ values can be decreased using acceptors with less spectral overlap. An additional opportunity provided by the use of coumarin probes is their high sensitivity to local polarity. The donor or acceptor emission may change in response to glucose binding due to relocation of the D or A, and thus provide a wavelength-ratiometric response. FIG. 45 at 2903 shows Glu-SF structures using a diboronic acid on a C6 linker with a collisional quenching mechanism. R is a long alkyl chain which may be the R chains shown in FIG. 42, or similar chains.

Alternate pairs which may be used as donors and acceptors and which would be suitable for creating a probe composition based on FRET include the following pairs (Donor sub-portion listed first, followed by the acceptor sub-portion:

Naphthalene paired with Dansyl;

Dansyl paired with fluorescein-5-isothiocyanate (FITC);

Dansyl paired with octadecylrhodamine (ODR);

1-N6-ethenoadenosine (ε-A) paired with NBD;

IAF paired with tetramethylrhodamin (TMR);

Pyrene paired with coumarin;

FITC paired with TMR;

5-(2-((iodocetyl)amino)ethyl)amino)naphthalene-1-sulfonic acid (IAEDANS) paired with FITC;

IAEDANS paired with 5-iodoacetamidofluorescein (IAF);

IAF paired an enzyme immunoassay (EIA);

carboxylfluorescein, succinimidyl ester (CF) paired with Texas Red (TR);

4,4-difluoro-4-bora-3a,4a-diaza-s-indacene (Bodipy) paired with Bodipy;

B-phycoerythrin (BPE) paired with a cyanine dye (Cy);

Terbium paired with Rhodamine;

Europium paired with Cy; and

Europium paired with allophycocyanin (APC).

### Example 24: Fluorescence Measurement System

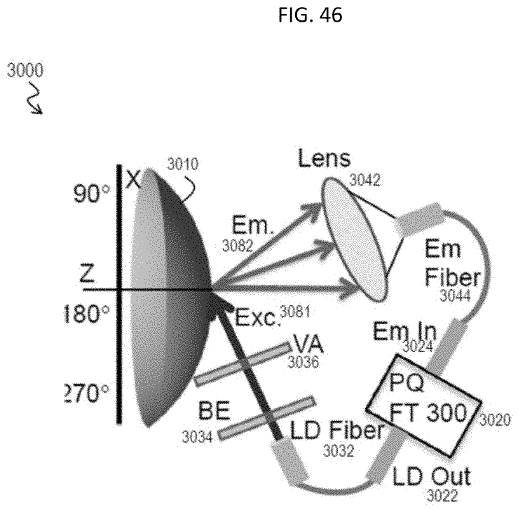

FIG. 46 illustrates an example measurement system 3000, according to an experimental embodiment. The system 3000 includes the probe material contact lens 3010 disposed on the cornea of a subject, with an axis of rotational symmetry defining the z direction, and the positive x direction at 90 degrees counterclockwise from the z axis. Tear fluid from the subject penetrates the contact lens 3010 and any analyte binds to the probe composition at the interface between the hydrogel channels and the silicone interstices.

An optical analyzer 3020, such as PicoQuant instrument (PQ FT 300 from PicoQuant Photonics International of West Springfield, Mass.) includes a pulsed laser diode (LD) that is energized to produce an laser diode (LD) source light that passes through LD output port 322 through optical fiber 3032 and optical couplers, such as beam expander (BE) and variable aperture (VA) 3036 to produce directional excitation light 3081 that impinges on the contact lens 3010. For example, the incident angle is selected to illuminate the contact lens 3010 at an angle to avoid direct incidence into the eye of the subject. The VA 3036 is used to illuminate part of the iris (1-2 mm diameter), the center outer surface of the pupil (1-2 mm), or the entire iris (about 10 mm) to obtain a maximal signal with minimal background.

The fluorescent emitted light, if any, emerges from the contact lens 3010 and is focused by an optical coupler, such as lens 3040, into the emission optical fiber 3044 where it is fed into the optical analyzer 3020 at optical input port 3024. The analyzer 3020 includes an LED source and control circuits, as well as an optical detector, analog to digital converter and Fourier analyzer. For example, the emission is collected with a 1-inch lens positioned about 1 inch from the eye, and focused into an optical fiber. The observation angle is close to perpendicular from the eye or slightly off the z axis. The emission is directed to the PicoQuant™ instrument for measurements of intensities, emission spectra or lifetimes. One can use light reflected or scattered from the eye as the timing reference for the lifetime analysis.

A digital input port (not shown) provides a digital signal from a separate processing system 380, such as a processor in a computer system 3100, chip set 3200 or mobile terminal 3300, or some combination, to control those components. A digital output port (not shown) outputs a digital signal carrying data that indicates the power fed into the LED and the spectrum of the received light. That output signal is received at the separate processing system 380 such as a processor in a computer system 3100, chip set 3200 or mobile terminal 3300, or some combination. At the processing system, the digital signal is used to determine the concentration of analyte in the tear fluid of the subject using a calibration curve appropriate for the probe material and detection method, as described above.

### Example 25: Z-Resolution Determination

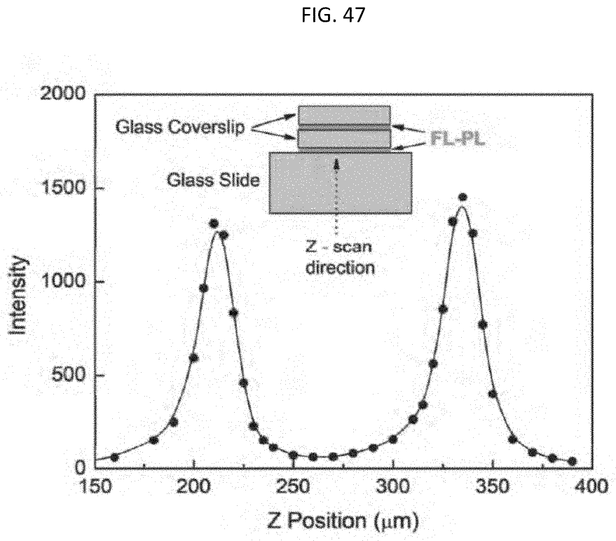

To test the z-resolution of the microscope, a geometric configuration of fluorescent films comparable to that expected from cornea and lenses was obtained using two cover slips in a small petri dish (FIG. 47). The thickness of a 123 μm cover slips are comparable to a contact lens. When two glass surfaces are coated with water, the spontaneous distance between the surfaces is 4 to 5 μm. The two cover slips were placed in an 8 μm solution of FL-PL in buffer pH 7.4. Z-scan intensities through the cover slips were measured using 473 nm excitation, 520 nm emission, using a 20× objective, NA 0.40, and a 50 μm pinhole. Two sharp intensity peaks were observed 130 μm apart, consistent with the expected z-axis FL-PC distribution for the 123 μm thick cover slip (see FIG. 47). The width of the peaks show that the z-axis resolution was near 21 μm, which is only 2-fold larger than the expected diffraction limit of 10.2 μm (see FIG. 3). The z-axis resolution can be increased using a smaller pinhole. In certain cases it is expected that 40× (NA=0.65) and 100× (NA=0.90) objectives are preferable, with z-resolutions of 3.91 and 2.04 μm, respectively. Using different optics the z-resolution should be increased by a factor of four to about 6 μm, if needed. The z-resolution using any of these objectives is more than adequate to resolve emission from each surface of a contact lens.

### Example 26: Posterior Surface Labeling

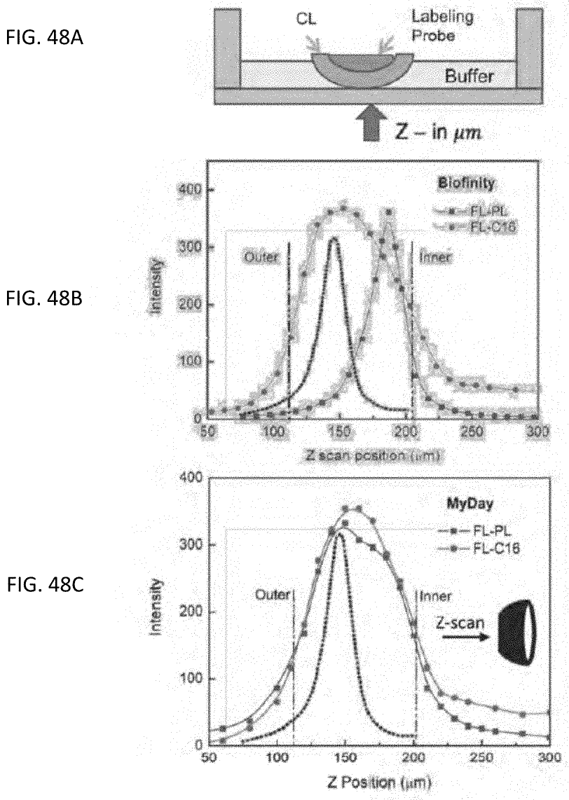

A Biofinity™ contact lens was labeled on the posterior surface by addition of 100 μl of 8 μM FL-PL. After 15 minutes, the lens was removed and washed with buffer. After rinsing, only one sharp peak of emission was observed on the back lens surface. See FIG. 48. This peak stayed sharp and remained on the back surface for at least 5 days. When the lens was completely immersed in a FL-PL solution, both sides were labeled and two peaks were observed (not shown). The z-axis intensity distribution has the same width as seen for the cover slip, showing that the width is a property of the LSFCM and the actual spatial distribution of FL-PL may be more localized at the surface.

Similar experiments were performed with the hydrophobic probe Fl-C 16, which is fluorescein covalently linked to a C18 alkyl chain. In this case a wide z-axis distribution was observed immediately after labeling and rinsing (FIG. 48). These results indicate a single C16 chain is not adequate to localize a fluorophore in a Biofinity™ lens. In other embodiments, with ISF-L longer and more hydrophobic linkers can be used, such as C20, two alkyl chains, cholesterol analogues or a phytol side chain which is a 12-carbon chain with four methyl groups. With more hydrophobic side chains, low water solubility may prevent the ISF with a linker (ISF-L) may prevent the ISF-L from reaching the contact lens during labeling. If this occurs, lyso-phosphatidylcholine (LPC) can be used as a carrier. Lyso-phospholipids are found in tears and used to increase the wettability of SiHG lenses.

The FL-PL was found to be uniformly distributed along the z-axis, which is through the contact lens. In this case, hydrophobic binding was not useful for surface labeling. The wide distribution of FL-C18 does not mean hydrophobic linkers cannot be used, because in other types of lenses several fluorophores with hydrophobic linkers were found to remain at the CL surface.

No ISF-L washout has been observed from the lenses over periods of weeks. If washout does occur, covalent linkage can be used to attach the ISF. This approach can be applied using probes which have a free amino group or which are linked to PL. Many contact lens polymers have net negative charges due to the presence of carboxyl groups from methylacrylic acid. The negative charge of lens polymers is demonstrated by the rapid absorption of lysozyme, which is one of the few proteins in tears with a net positive charge. Covalent binding can be performed after the ISF-L is bound to the lenses. The lens and ISF-L can then be covalently linked with N-(3-dimethylaminopropyl)-N′-ethylcarbodiimide hydrochloride (EOC) and N-hydroxysuccinimide (NHS). After the reaction, the lenses can be washed and base hydrolysis used to eliminate the remaining activated carboxyl groups.

A similar procedure can be used with hydrophobic side chains. In this case the side chains will contain amino groups which are located close to the ISF-L. These ISF-L can bind due to hydrophobic interactions and then react covalently with activated carboxyl groups. The conditions and coupling reagents can be changed by a person of ordinary skill for efficient coupling. The covalently labeled lenses also can be examined by LSCFM and the reaction conditions adjusted, if necessary.

### Example 27: Covalent Labeling

The carboxyl groups on the MyDay™ lenses were activated using EDC/NHS (1-ethyl-3-(3-dimethylaminopropyl)-carbodiimide/N-hydroxy succimide) chemistry. After activation, the amine-containing fluorophore fluorescein-cadavarine (Fl-CD) was added to the EDC/NHS activated lens and to a control lens not treated with EDC/NHS. The lenses were washed extensively for 5 days after which the activated lenses displayed a high fluorescein emission and the untreated lens was almost non-fluorescent (not shown). This procedure can be used to combine the surface localization of polylysine with covalent linkage to the ISF. Since Fl-PL binds to the surface, the carboxyl groups can be activated with EDC/NHS before or after FL-PL binds to the lenses.

### Example 28: Pairs of Ion-Selective Fluorophores for Use in Contact Lenses

Certain pairs of fluorophores suitable for measurement of ion concentrations in the PLTF and PoLTF. Suitable fluorophores have several characteristics and are not directly available from commercial vendors. The first characteristic is that the pair of fluorophores are sensitive to the same ion in two separate locations. Pairs of fluorophores for the same ion are generally not described. Suitable ISF also have ion binding affinities within the physiological range of electrolytes in tears (see Table 1). Additionally, the ISF binds tightly to the contact lens without washout from tear replacement for hours or days of use. The binding is typically provided by a ligand (L) which has high affinity to the lenses. Of course, the ISF-L is useful with wavelengths in the visible to NIR range, is photostable, and is non-toxic in the amounts which potentially can be released from the lens. Finally, the fluorophores display a change in lifetime upon changes in ion concentrations, or contain a non-ion sensitive fluorophore for wavelength-ratiometric measurements.

Synthesis and development of ISF-L probes which satisfy these requirements is in its infancy. It is already known that the binding affinities of ISF can be different for the fluorophore in buffer compared to the unique environment at the interface region of lenses. This effect may be different in different contact lenses, and a single ISF-L may not be useful in all lenses. Some potential ISF pairs for each of the ions are found in Table 2.

### Example 29: pH Measurements in the PLTF and PoLTFs

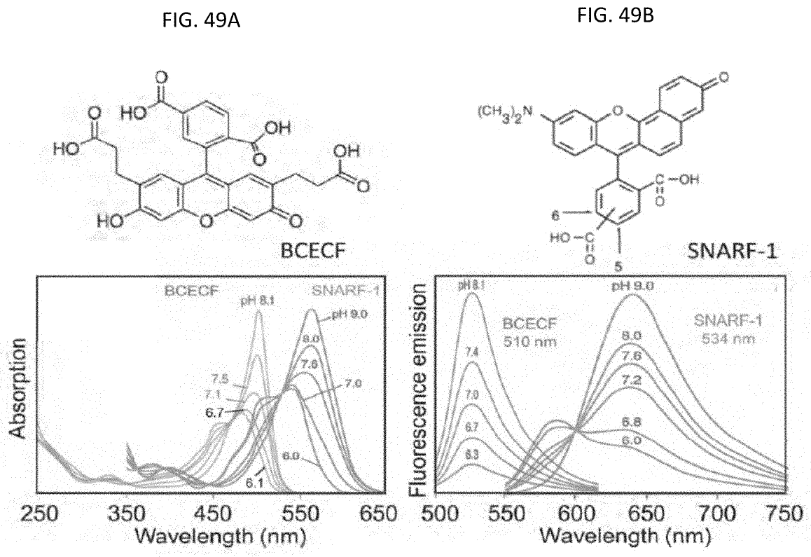

One pair of probes for pH measurements is a fluorescein derivative BCECF and a rhodamine derivative SNARF-I (see FIG. 7). This pair is described here in more detail to illustrate the needed spectral and chemical properties. The probes have pKa values close to the range of pH values found in tears (Table 1), these are 7.4 for BCECF and 7.5 for SNARF-1 (FIG. 49). BCECF has been studied extensively for sensing in tissue cultures. Both probes can be excited in the same wavelength range from 460 nm to 530 nm. The emission spectra are widely separated. BCECF can be selectively observed at 525 nm and SNARF-I at 650 nm. This pair of probes have favorable features for sensing in lenses. BCECF can be used as a wavelength ratiometric probe using different excitation wavelengths to provide two measurements of the pH. The intensity of BCECF increases about 8-fold from low to high pH and the lifetime changes from 3.0 to 3.9. This lifetime change is suitable for lifetime-based sensing (LBS). SNARF-1 has the same advantages as BCBEF. The intensity changes about 5-fold and the mean lifetime at 650 increases with pH from 0.94 ns at low pH to 1.45 at high pH. SNARF-6 bas a chemical structure very similar to SNARF-1, and displays a larger lifetime change from 0.94 to 4.51 ns from low to high pH. Because the emission spectrum shifts with pH, SNARF-1 also can provide wavelength-ratiometric measurements of pH.

Many pH probes are commercially available, typically in both the water soluble (WS) and cell permeant (AM) forms, from companies such as Invitrogen-Molecular™ Probes and AAT Bioquest™. But most pH SNARF-1 probes are not available with reactive side chains for covalent binding to linkers. For applications suitable for use with preferred embodiments of the invention, the most important property is a pH-dependent spectral shift or change in lifetime, to allow wavelength-ratiometric or lifetime-based sensing. The initial choices for a suitable pH probe are fluorescein derivative BCECF and rhodamine derivative SNARF-1. The probes have pKa values close to the range of pH values found in tears (7.4 for BCECF and 7.5 for SNARF-1. BCECF has been studied extensively for sensing in tissue cultures. Both probes can be excited in the same wavelength range from 460 nm to 530 nm. The emission spectra are widely separated. BCECF can be selectively observed at 525 nm and SNARF-1 at 650 nm. This pair of probes have favorable features for sensing in lenses.

BCECF can be used as a wavelength-ratiometric probe using different excitation wavelengths. The intensity of BCECF increases about 8-fold from low to high pH and the lifetime changes from 3.0 to 3.9. This lifetime change is suitable for lifetime-based sensing (LBS). When both probes are fluorescent ion concentration transients could be detected by different measurements at two wavelengths, as we have done with frequency and time-domain measurements. SNARF-1 and SNARF-6, which differs from SNARF-6 by one methyl group, have the same advantages as BCBEF. The intensity changes about 5-fold and the mean lifetime at 650 nm increases with pH from 0.94 ns at low pH to 1.45 at high pH. SNARF-6 displays a larger lifetime change of 0.95 to 4.51 ns. The large pH dependent shift in the emission spectrum allows SNARF-1 and SNARF-6 display different apparent pKa depending on the observation wavelength, from 6.6 at 640 nm to 8.3 at 580 nm. This change occurs because the lifetime is an intensity weighted value. The actual pKa does not change. The ability to choose the chosen apparent pKa can be used to keep the probe response in the optimal physiological range. The SNARF dyes are derivatives of rhodamine and are not expected to be toxic to eyes.

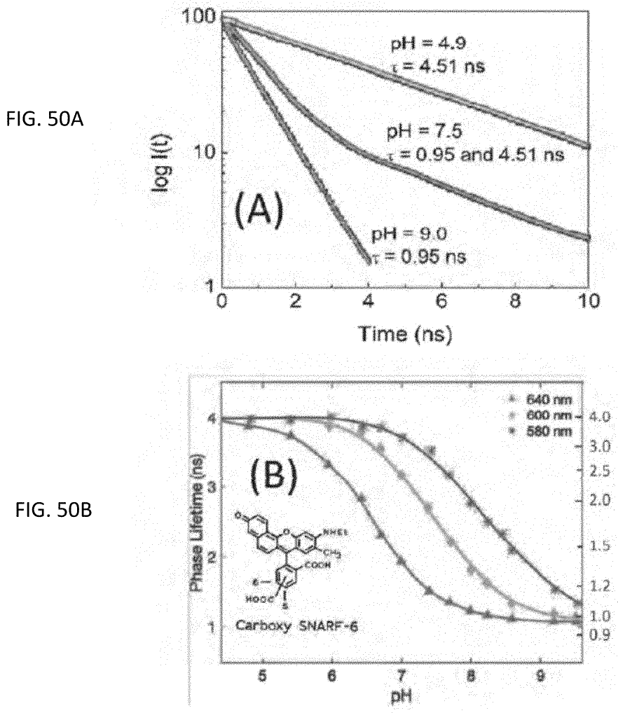

As mentioned above, the local environment at the contact lens surface can affect the dissociation constant for proton or metallic ligands. The use of SNARF-1 or SNARF-6 provides an approach to adjusting the ion-sensitive range to the physiological range. This change is possible by selection of the observed emission wavelength. Because both forms of the SNARF probes (the low and high pH forms) are fluorescent, the intensity decays can be multi-exponential at intermediate pH values (see FIG. 50A). The observed fractional emission from each form results in a different intensity decay at each wavelength. As a result, the apparent pKa can be selected by changing the observed emission wavelengths (see FIG. 50B). The intensity decay displays two decay times 4.9 ns and 0.95 ns, assigned to the low and high pH form, respectively.

The relative contribution to the intensity decay depends on the observation wavelength. The apparent pKa can be changed from near 6.0 at 640 nm to 8.0 at 580 nm (FIG. 50B). This change occurs because the lifetime is an intensity weighted value. The actual pKa does not change. The ability to choose the chosen apparent pKa can be used to keep the probe response in the optimal physiological range. The BCECF and SNARF dyes are derivatives of fluorescein and rhodamine and are not expected to be toxic to eyes. The chemical structure shown in FIG. 7 are not adequate as a ISF-L for pH. The structure requires attachment of a ligand for binding to the lenses, and in the case of BCECF removal of the acetoxymethyl (AM) groups. The use of SNARF-1 will require adding a ligand to one of the free carboxyl groups.

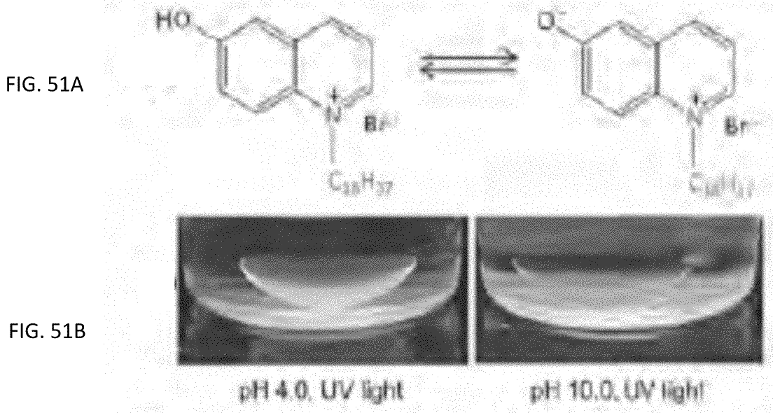

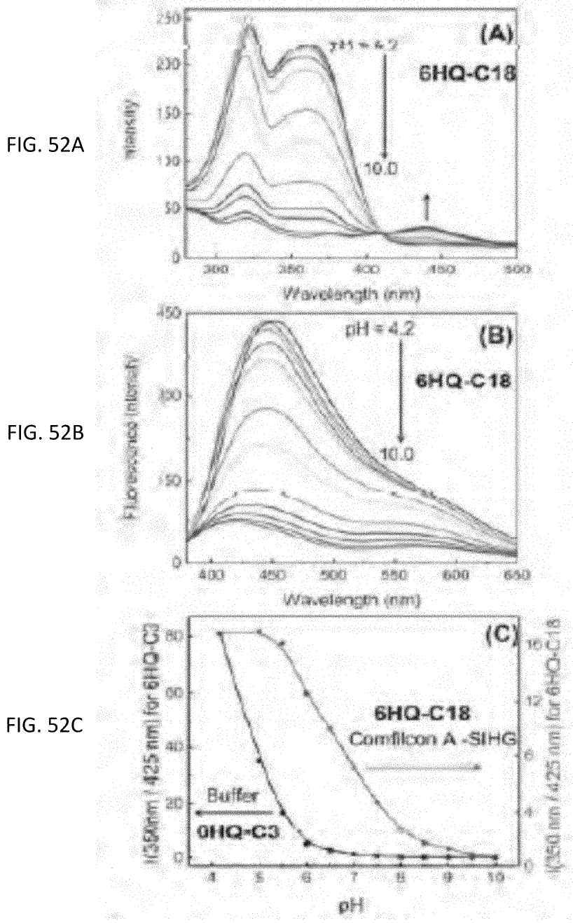

Initial work on a pH-sensitive contact lens was published previously and used a hydrophobic side chains to bind to the non-polar silicone phase in Biofinity™ SiHG contact lens. The pH sensing moiety was 6-hydroxyquinoline. This group was reacted with 1-bromo-octadecane to form 6HQ-C18 (FIG. 51A). This probe bound quickly to the Biofinity™ lenses and displayed bright pH-dependent fluorescence which could be seen in room light (FIG. 51B). The 6HQ-C18 could not be washed out of the lens for weeks after placing into fresh buffers. As the pH increased the emission turned from a bright blue to a less-bright yellow. Absorption and emission spectra were recorded for pH values from 4.2 to 10.0 (FIG. 52). The absorption changed with increasing pH, and the emission intensity decreased using the ratio of 350 nm absorption and the emission intensity at 450 nm. This ratio deceased with increasing pH. Comparison of the lens labeled with 6HQ-C18 with water soluble 6HQ-C3 in buffer revealed the pKa of 6HQ-C18 was increased by at least 3 pH units upon binding to the lens, and the higher pH response was in the physiological pH range. This result illustrates the known ISF binding constants in buffer can be changed by interaction with a contact lens. Thus, the person of skill can synthesize similar but new structures to produce probe compositions with sensitivity in the physiological range, if needed.

### Example 30: Sodium and Potassium Sensing

Measurements of Na⁺ or K⁺ concentration by fluorescence are more difficult than pH. In the case of pH sensing, a weak covalent bond is broken when dissociation of a proton occurs. This dissociation can change the hybridization of electron orbitals in the fluorophore, and thereby change its absorption and emission spectra. Only ionic bonds and non-covalent bonds are formed by Na⁺ and K⁺. As a result, the fluorophore needs to respond to the near field around the ion, typically by ion binding to a chelator which in turn affects the fluorophore.

Many sodium-sensitive fluorophores (SSF) are known in the art. The features beneficial in an SSF for use in a contact lens include the ability to distinguish the two SSF from the spectral data and to have sodium affinity constants in the physiological range for tears. Almost all SSF are based on azacrown ethers which are attached to the fluorophores by the one or two azacrowu nitrogen atoms. The first reported SSF useful in cells was sodium binding benzofuran isophthalate (SBFI). SBFI required UV excitation wavelengths from 320 to 380 nm. These short wavelengths could result in high autofluorescence from the eyes and could result in UV photodamage.

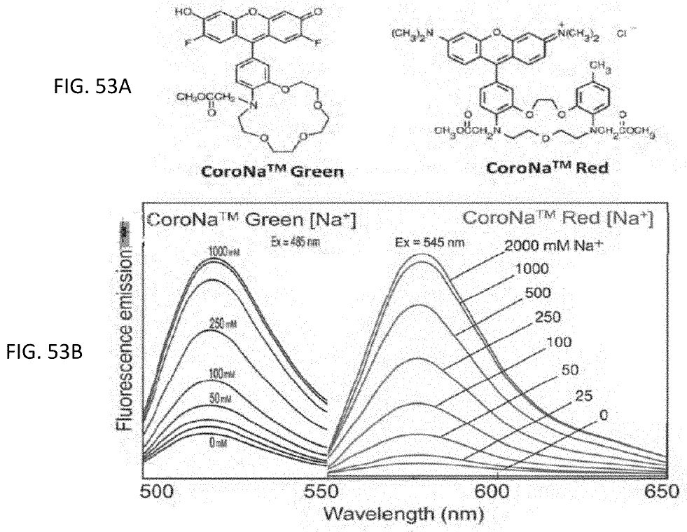

CoroNa Green and CoroNa Red were selected for study here. Both probes can be excited with visible wavelengths near 450 nm. See FIG. 53. The emission spectra are widely separated with peaks at 520 and 580 nm, and the probes can be selectively observed at these wavelengths. An important property of these SSF are the binding constants, which are reported to be 82 mM for CoroNa Green and 200 mM for CoroNa Red, and which are ideal for sodium sensing in tears. CoroNa Green can be used for LBS because multi-exponential decay in the absence of Na⁺ which is about 2-fold faster than the mono-exponential decay when bound to Na⁺. Lifetime data was not found for CoroNa Red but from the intensity increase when bound to Na⁺ it is likely that the lifetime also increases with Na⁺ binding. This pair of probes is probably useful for Na⁺ measurements in the PLTF and PoLTF. The probes cannot be used directly but require covalent linkage to polylysine for contact lens surface localization. This can be accomplished by starting with the water-soluble version of the probes, without the acetoxymethyl ester (AM) group, which is used to make the probes permeable to the cell membrane. If only the AM probe is available, the esters can be hydrolyzed to obtain free carboxyl groups for conjugation with poly-lysine or a hydrophobic linker.

### Example 31: Na⁺ Sensitive Contact Lenses

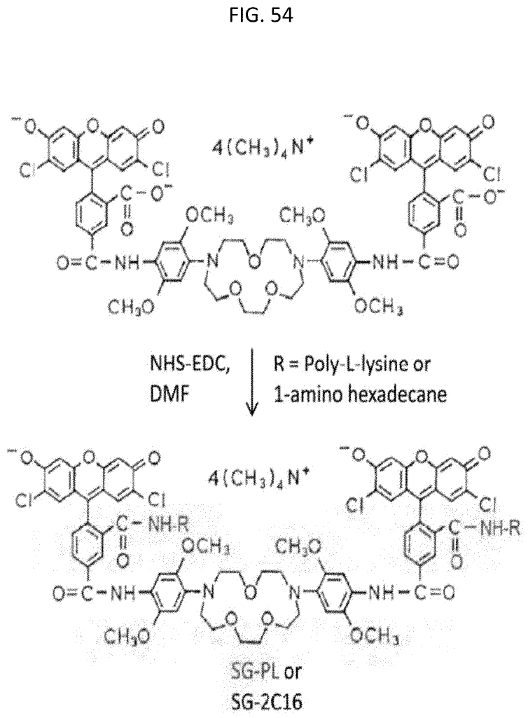

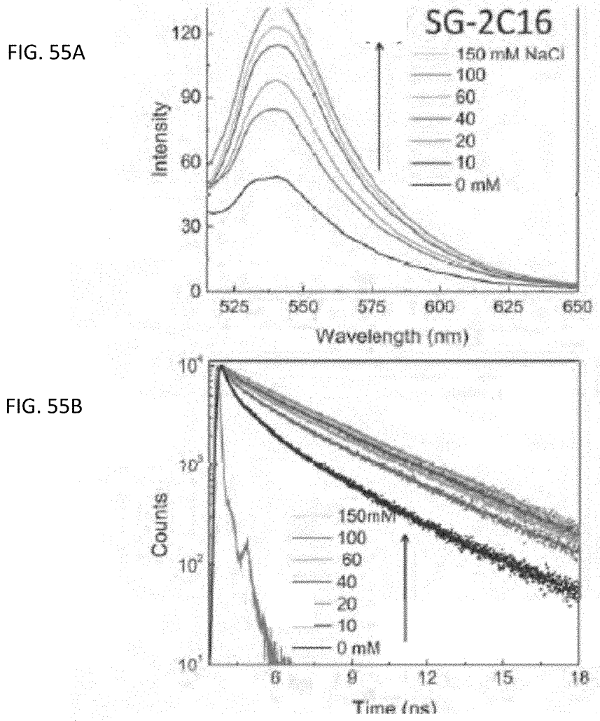

In an Na+-sensitive contact lens based on Sodium Green (SG), the 1SF SG was covalently linked to polylysine (PL) or a C16 chain (see FIG. 54) which rapidly bound to both Biofinity™ lenses and more slowly to MyDay™ lenses. Binding of Na⁺ to the labeled lenses resulted in an approximate 3-fold increase in intensity and lifetime (see FIG. 55A). The SG emission spectrum did not shift upon Na⁺ binding, but the lifetimes could be used to measure the Na⁺ concentration (see FIG. 55B). The experiments with SG demonstrate another possibility which is imaging the entire contact lens by intensity or lifetime. The use of FLIM will allow changes in ion concentration to be measured in different regions of the cornea.

The binding constants of SG-labeled lenses can change dramatically upon binding to a contact lens. SG itself in buffer solution displays a Na⁺ binding constant near 10 mM, so that SG would be fully saturated in tear fluid (132 mM Na⁺). However, addition of the linker and/or binding to the lenses decreased the SG binding constant to near 100 mM and useful in tear fluid.

### Example 32: Potassium Binding Compounds

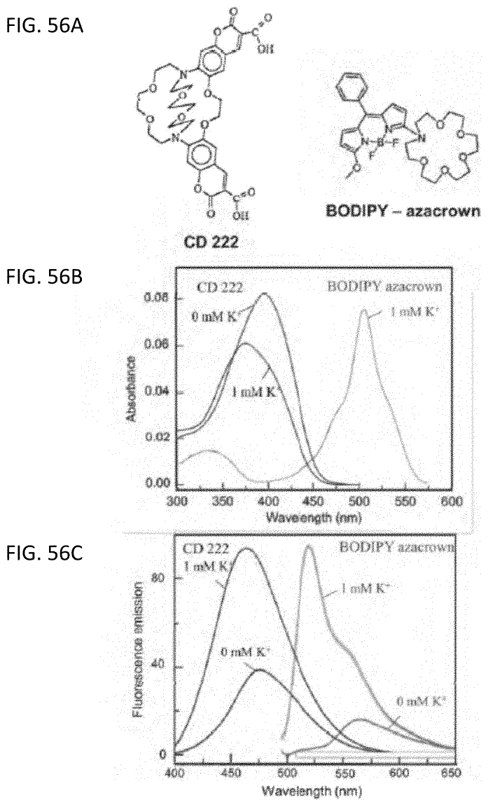

Potassium concentrations ion tears are 4 to 8-fold higher than in blood (see Table 1), which suggests a physiological importance for this ion. Fewer sensors are known for K⁺ than for Na⁺, however. K⁺ sensing is also complicated by interference by Na⁺ because the smaller Na⁺ ion can fit into azacrown compounds designed to bind K⁺. Specificity for K⁺ was increased by using the more rigid azacrown ion CD222 (see FIG. 56). CD222 absorbs light above 400 nm which may be safe in clinical measurements with an emission maximum near 455 nm. A spectrally different K⁺ probe (a BODIPY-azacrown whose emission maximum is about 550 nm, which is 80 nm longer than CD222) also can be used. The binding constants of CD222 and BODIPY-azacrown are less than 1 mM for K+, which is too strong to measure the 5 mM K+ concentration in tears. K+ binding may become weaker when the ISF are modified for use in lenses, as occurred for SG.

### Example 33: Calcium Ion Sensing

Fluorescence sensing of Ca⁺⁺ and Mg⁺⁺ generally is easier than the monovalent cations because the higher charge density results in larger spectral changes. The initial interest in Ca⁺⁺ sensing was focused on intracellular concentration because Ca⁺⁺ is involved in cell signaling through the membrane to result in activation of intracellular pathways. The intracellular concentrations of Ca⁺⁺ typically are near 100 mM and the Ca⁺⁺ probes such as Fura-2 and Quin-2 were designed to have dissociation constants near 200 mM. Because of the high positive charge on Ca⁺⁺, these probes displayed changes in their excitation or emission spectra upon Ca⁺⁺ binding. However, the Ca⁺⁺ concentration in tears is much higher (near 0.8 mM) and about 4000-fold more than the intracellular concentration. Therefore, lower affinity Ca⁺⁺ probes are preferred.

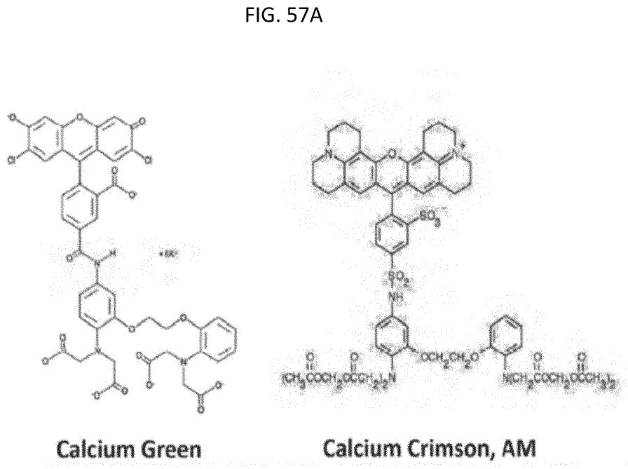

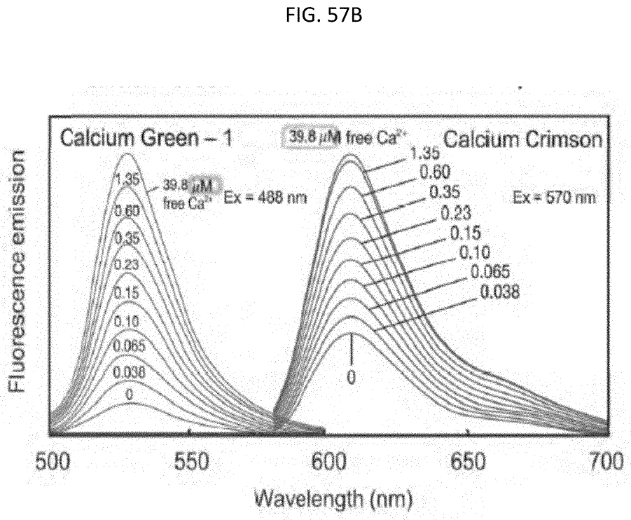

Calcium probes with lower affinity are known. One suitable pair of probes useful in embodiments for the PLTF and PoLTF are Calcium Green and Calcium Crimson (CC). These compounds are based on fluorescein and rhodamine derivatives, respectively, and display widely different emission maxima (530 and 620 nm) so that they can be observed separately. See FIG. 57.

For both of these probes, the emission intensities and quantum yields are increased from 10-fold to 3-fold upon Ca⁺⁺ binding. However, CG and CC do not display changes in their emission spectra, so that intensity-based measurements will not be useful in a clinical application. Fortunately, these probes display significant changes in lifetime which allows the Ca⁺⁺ concentration to be determined by LBS. CG and CC display only modest Stokes' shifts, and their absorption at wavelengths below the long wavelength absorption maxima are typically 10-fold or more lower than the long wavelength maximum. The person of skill can determine whether, for a particular embodiment, it is practical to excite both probes using a single excitation wavelength or if two excitation wavelengths are necessary. Either is contemplated for use in the invention. The lifetime of CG increases from 1.38 at 0.0 mM Ca⁺⁺ to 3.61 at 1.35 mM Ca⁺⁺. For CC, the lifetime change is smaller, from 2.56 to 4.10 ns, which is adequate for LBS in both tear films. The Ca⁺⁺ binding constants from steady state measurements are 0.128 and 0.283 μM for CG and CC, respectively, which is much lower than the Ca⁺⁺ concentration in tears. The binding constant can increase to the 0.8 mM range in tears, as occurred with Sodium Green.

### Example 34: Magnesium Ion Sensing

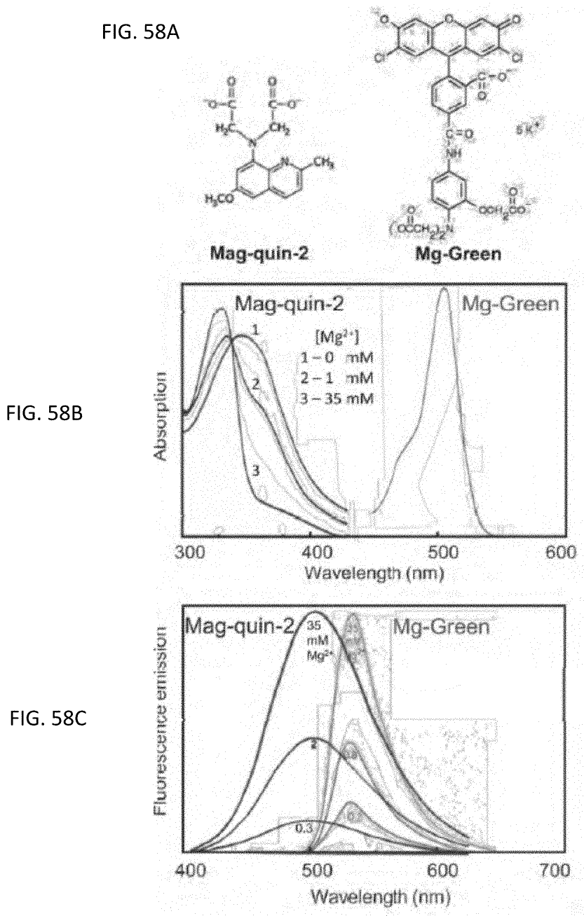

There are fewer probes for Mg⁺⁺ than Ca⁺⁺. One potential pair for the two tear films is Mag-quin-2 and Magnesium Green (MG). One design principle for a suitable Mg⁺⁺ probe is typically to have one less carboxyl group for Mg⁺⁺ probes than for Ca⁺⁺ probe (see FIG. 58). The most practical and commercially available Mg probe contemplated for use in the invention is MG, which contains the same chlorinated fluorescein used in CG. In contrast to the Ca⁺⁺ probe, a longer wavelength Mg⁺⁺ probe is not readily available. The second Mg⁺⁺ probe can be Mag-quin-2, for which the emission maxima overlaps with MG. The signal from Mag-quin-2 can be separately observed for wavelengths from 410 to 490 nm. In contrast to the Ca⁺⁺ probe, the emission spectra overlap and MG cannot be observed without a contribution from Mag-quin-2 (see FIG. 58), but these signals can be separately quantitated by a multi-exponential analysis of the emission from 500 to 600 nm.

Because of correlation between the amplitudes and lifetime, it can be difficult to resolve multi-exponential decays. For the suggested pair of Mg++ probes, this resolution can be improved by the use of Global analysis, which requires one of the parameters to be the same for multiple sets of data. For the two Mg⁺⁺ probes the intensity decays from observation at 450 and 520 nm can be analyzed while the lifetime at 450 nm is held constant, as one of the decay times at 520 nm. A similar approach is to use the 450 run decay time as a fixed parameter during analysis of the 520 nm emission. These considerations indicate the fluorophore pairs with overlapping spectra can still be used to quantify concentrations in the two tear films. The dissociation constant for Mag-Quin-2 and MG, 1.00 and 1.4 mm, respectively, are close to the physiological concentration in tears (see Table 1). The lifetime of MG changes from 1.22 to 3.68 as the calcium concentration is increased from 0 to 35 mM Mg⁺⁺. The intensity decay of Mag-quin-2 is strongly multi-exponential, but the mean decay changes from 2.0 to 8.56 from 0 to 35 mM Mg⁺⁺. Because of large changes in the absorption spectrum due to Mg⁺⁺ using the phase-modulation method can be changed from 0.30 to 40 mM depending on the excitation wavelength.

### Example 35: Chloride Sensing

Injury to the cornea is expected to result in changes in electrolyte concentrations and the largest change may occur for chloride. Higher [Cl⁺] in blood or tears appears to be linked to cystic fibrosis. The selection of two different fluorophores sensitive to chloride is much simpler than the other ions because chloride sensing does not involve binding to the fluorophore. Two different chloride probes are shown in FIG. 59.

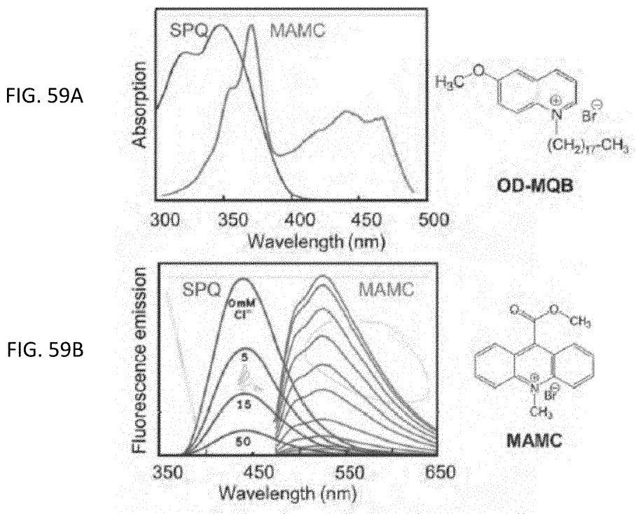

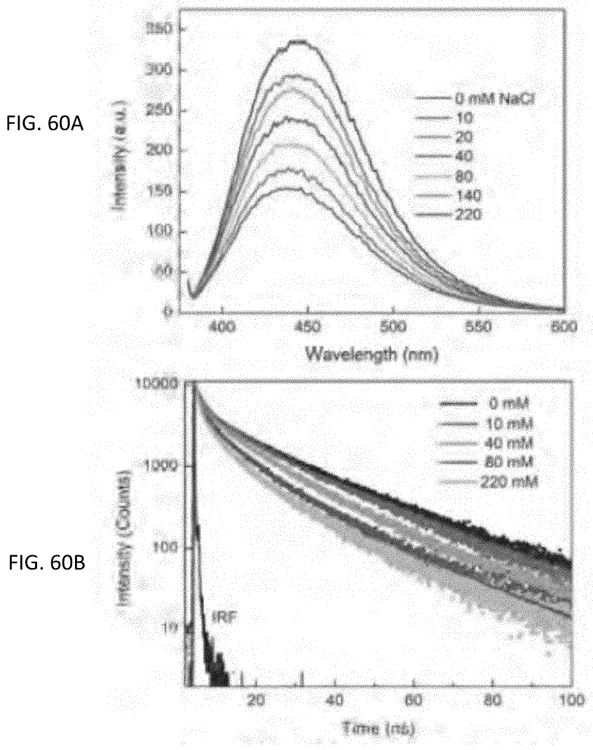

The OD-MQB probe has two rings, and MAMC has three rings, all electron deficient. These probes can be selectively observed at 440 nm for OD-MQB and at 550 nm for MAMC. Both probes can be excited with incident light near 400 nm. In the excited state the probes are quenched (returned to the ground state without emission) upon contact with chloride. OD-MQB was efficiently quenched when hydrophobically bound to MyDay™ sensitive fluorophores (see FIG. 60). The probes are not destroyed by quenching but remain available for further excitation and quenching. The intensities (I) and lifetimes (τ) are analyzed using the Stem-Volmer equation F₀/F=1+K[Cl⁻] or τ₀/τ It=1+K[Cl⁻] where F₀ and τ₀/τ are the intensities and lifetimes in the absence of chloride, and K is the quenching constant and the inverse of the [Cl⁻] needed for 50% quenching. For collisional quenching the value of F₀/F and are typically the same. For these chloride probes, the emission spectra do not shift on quenching. Any difficulties associated with this can be avoided by using lifetime-based sensing, or by using a reference fluorophore and the wavelength-ratiometric method. One example of such a probe is an SPQ analogue covalently linked to a fluorophore which is not sensitive to Cl⁻ quenching. The fluorophore not sensitive to chloride has an emission maximum at 50 nm which can be readily measured separately from the SPQ-like emission at 450 nm. Chloride concentrations in lenses can be determined by intensity or lifetime measurements. A favorable result is very similar quenching in the Biofinity™ and MyDay™ lenses (FIG. 9) which suggests the fluorescent part is completely in the aqueous phase. Hydrophobic binding to MyDay™ lenses is favorable for absorption and emission spectra.

### Example 36: Contact Lenses for Multiple Ionic Species

Research or clinical applications of the inventive methods are contemplated to use measurements of more than a single ionic species. At present, the measurements here have been limited to contact lenses labeled uniformly across the surface, in the x-y plane. For uniformly labeled lenses six ISF-L will be required to determine the ions in tears (see Table 2). If measurements of both the PLTF and PoLTF are required, then 12 spatially distinct ISF-L are required.

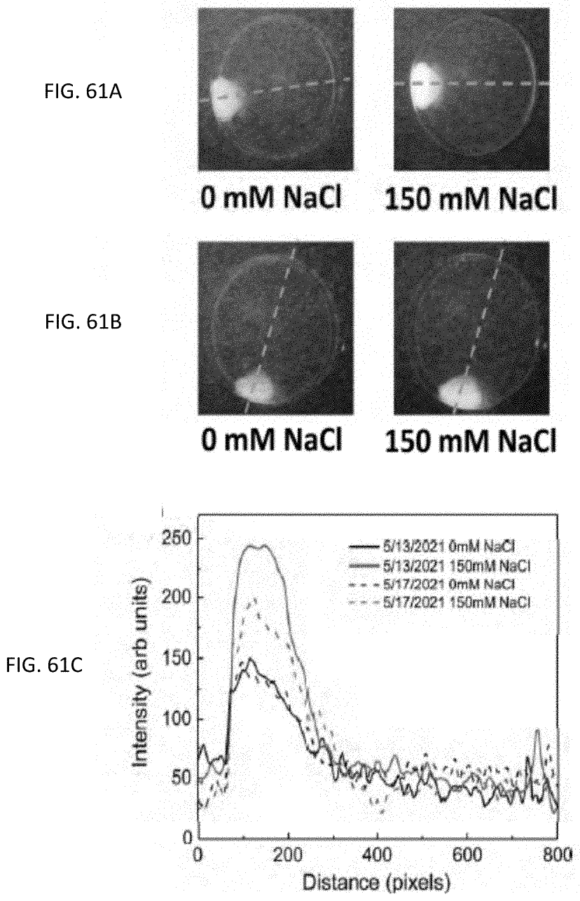

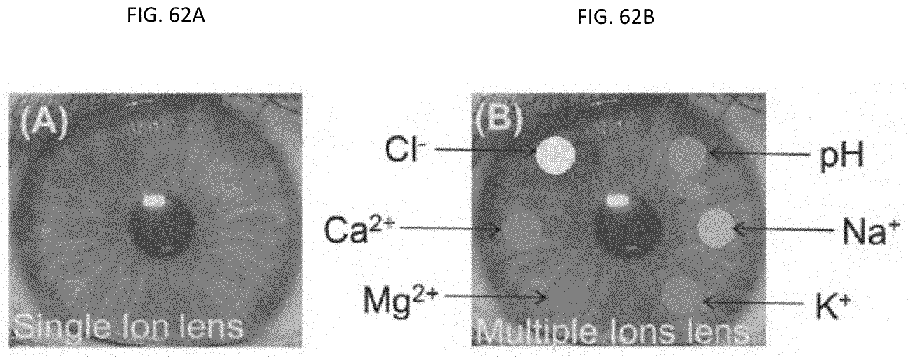

Difficulties that may be involved in embodiments involving the selective detection of 12 different can be solved if the ISF-L are localized at distinct locations on the lens surface. This approach requires that the ISF-L do not migrate by lateral diffusion across the lens surface. The presence or absence of lateral diffusion was tested using a fine needle to spot a small location on the edge of a contact lens. A Biofinity™ lens was spotted with the sodium sensor SG-PL (see FIG. 54) in the absence or presence of 150 mM Na⁺. The fluorescence was photographed immediately after probe binding (FIG. 61A) and after incubation in buffer for 4 days (FIG. 61B). The images were essentially identical. The distribution was quantitated by intensity line tracing along the dashed red lines (FIG. 61C), which showed no increase in spot size. The spot size can be smaller and more controlled with an automated spotter. The lack of lateral diffusion shows that the multiple lens shown in FIG. 62B can be fabricated with ISF-L for the electrolytes in tears.

### Example 37: Toxicity Testing

Poly-lysine (PL) is not expected to be toxic in the contact lenses and can be used as a linker. The amounts of PL bound to the lenses are small, but do not experience washout of probe-poly-lysine conjugates from the Biofinity™ and MyDay™ lenses. PL MyDay™ has been used for over 40 years in cell biology studies. Glass slides are when immersed in a PL solution. Many types of cells rapidly grow on these surfaces. PL serves as an antibacterial agent on instruments such as catheters. Many companies sell sterile solid PL and PL solutions for the purpose of making surfaces biologically friendly with PL molecular weights ranging from 3 KDa to over 300,000 KDa. Dendritic PL and epsilon poly-lysine are also available. PL dendrimers are used for drug delivery using eye drops. Therefore, PL is not expected to be toxic in use for this invention.

The Draize Eye Test (DET) in rabbits is the gold standard for eye toxicity testing. This procedure is painful to the New Zealand White (N2W) rabbits involved in the study. The DET is required is some countries and banned in others for testing of cosmetics and many alternative tests have been proposed. At present, the most used test procedures consist of a layer of corneal cells grown in vitro on a nanoporous substrate so the cells are in contact with culture medium on one side and air on the other side. Two cell lines which are suitable for use: engineered human-derived epidermal keratocytes (i.e., Epi-Derm™ from MatTek™) and cultured human corneal cells (Epi Corneal™ from MatTek™). At first glance, the human corneal cell model seems superior (Epi Corneal™) but the Epi Derm™ test has been more extensively tested against the DET. Either test can be used here to confirm non-toxicity of the lens materials for use in the invention.

The type of test used can be selected easily be the person of ordinary skill, or by consultation with an ophthalmologist. These tests are expensive but the per-chemical cost decreases rapidly with the number of chemicals to be tested. Test of one chemical costs about $3000, five chemicals about $5000 and 10 chemicals about $7000. These costs sound expensive but are far less than the cost of 1 or 2 molecular biologists, setting up a sterile hand and an incubator for CO2 control. Also, testing in such a laboratory would require extensive pre-testing in unsure correct results. Many companies have selected MatTek™ for this testing. The testing can be performed in several ways, for example using particles made from the labeled lenses or the PL-probe itself. Direct placement of the probe on the corneal cells for testing is more sensitive, but such contact is more inconvenient with labeled lenses.

### Example 38: Fluorescence Testing in Rabbit Eyes

Fluorescence measurements are often limited by background emission or light scattering from the sample. The background and scatter from tissues is usually much greater than from samples in cuvettes or on slides. For this reason, the emission from a contact lens with Sodium Green-polylysine (SG-PL) was measured when placed on a rabbit eye. The optical configuration was similar to the expected configuration in a clinical setting (FIG. 46). The incident light at 473 nm was at an oblique angle near 30° to avoid direct entry of bright light into the eye. Because the cornea and lens are transparent, a high scattered light intensity into the eye was not expected.

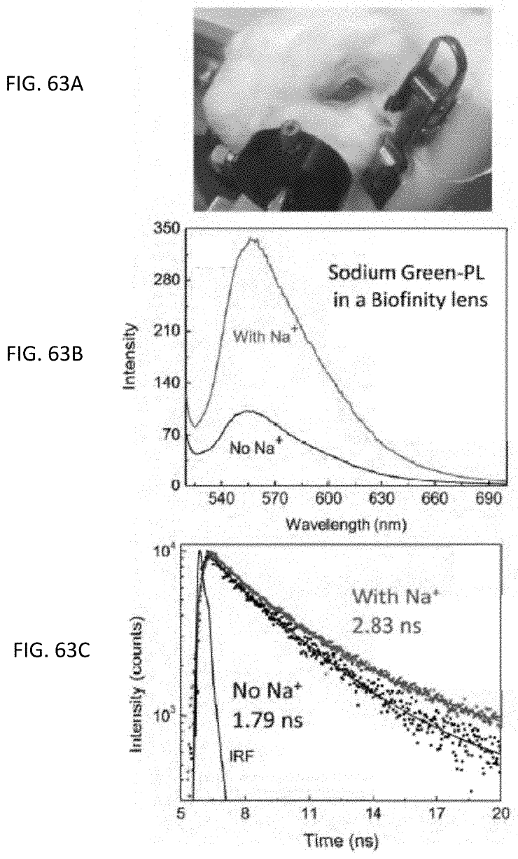

The emission signal was sent to the time-resolved spectrofluorometer (described herein) by a filter optic cable. The white New Zealand rabbit was not alive, which should not have affected the results. The emission spectrum was easily measured and the shape indicates no substantial background from the rabbit eye (see FIG. 63). The steady state emission and intensity decay were both responsive to the Na concentration in a manner comparable to SG-2C18 in FIG. 27 and FIG. 28. Exact agreement was not expected because the linkers are different.

### Example 39: Testing of Contact Lenses Using Ex-Vivo and In-Vivo Rabbit Corneas

In order to determine if the surface-labeled lenses can be detected without excessive background emission from the eyes or cornea, testing is performed first to determine if the lenses respond to changes in the ion concentrations in the PLTF and PolTF. Cornea and eye samples from White New Zealand rabbits traditionally are used for eye research because the tear fluid is similar to humans and their eyes are similar in size and shape to human eyes. Thus, they are suitable for testing here. They also are known to be well adapted to animal husbandry conditions. When needed, the animals will be anesthetized as described in the VAS, and to minimize discomfort the lenses will remain on the rabbit eyes only during the experiments, and not for longer periods of time.

For the testing, contact lens is inserted in one rabbit eye with a solution that mimics the composition of tears and is continually flushed with that solution, except for variation in one of the ion concentrations. A flow rate of 0.5 μL/min, which, is 10-fold larger than the tear production rate in humans (near 0.05 μL/min for normal individuals) is suitable. Twelve (12) different ion concentrations are tested, the rabbit allowed to return to baseline, then the 12 ion concentrations tested a second time. These preliminary in-vivo tests are to determine if both the PLTF and PolTF can be observed and to determine the response to ions.

Ex-vivo experiments are performed using mature rabbit eye corneas and whole rabbit eyes. The optical configurations are shown in FIG. 46. The measurements can be at a single location in the cornea or eye, or the incident beam can be defocused to obtain a large average signal from the labeled lens. These instruments do not use confocal optics so the incident light and emission will be from the entire lens plus cornea thickness. The second type of measurement will be LSCFM confocal FLIM Imaging of the lens and cornea samples. Both intensity and lifetime (FLIM) images are collected from the lens, the cornea, and the lens plus cornea sample to determine if background contributes to the total emission from the samples.

Rabbit heads, corneas, and eyes, are commercially available. Corneas are mounted on glass or quartz hemispheres to maintain cornea shape and kept moist in a saline bath to cover the bottom of the chamber with a cover used to maintain humidity. In these ex-vivo experiments, there is no tear fluid to form a layer and the lenses may not be retained. Pins or the eyelids will be used to restrain the lenses. In the absence of a tear film, the FL-PL binds directly to the corneal epithelial cells. Such binding is probably due to the negative charge on cell membranes. The cornea and lens samples are bathed in solution with electrolytes (Na⁺, H⁺, or Cl⁻) at the desired concentration. Both single point measurements and confocal images are taken to determine if both the PLTF and PolTF labels are detectable and if the probes respond to ion concentrations.

In one test, a fresh rabbit head was obtained from a local source. A Biofinity™ lens was labeled on both sides by immersion in a 1 μM solution of SG-PL in buffer for 10 minutes followed by extensive rinsing. This lens was placed on the rabbit eye and held in place by the eyelids. The emission from SG-PL was observed using a fiber optic configuration. The emission from SG-PL was readily observed without significant background emission from the rabbit eye (see FIG. 60). Changing the Na⁺ concentration from 0 to 150 mM resulted in a 3.4 fold increase in emission intensity and an increase in lifetime from 1.79 to 2.83 ns. These spectral changes are in close agreement with the changes observed for SG-PL in a Biofinity™ lens observed in buffer solution (FIG. 26). FL-PL could not be washed out of the eye, suggesting it bound to the cell membranes.

### Example 40: Instruments for Measuring the PLTF and PolTF Concentrations

Small simple instruments can be constructed for single point or imaging measurements. All the electronics for time-resolved (TCSPC) measurements are now available as a single computer board. Continuous and pulsed LED and laser diode light sources are available for wavelengths from the UV at 260 nm to above 900 nm in the NIR. Charged-coupled devices (CCDs) are rapidly being replaced by CMOS detector arrays (CDAs) with high sensitivity and frame rates. Their sensitivity is adequate for single molecule detection. The CDAs are also capable of FLIM measurements without the use of additional gating devices.

### REFERENCES

All references listed below and throughout the specification are hereby incorporated by reference in their entirety.
1. Phadatare, S. P., Momin, M., Nighojkar, P., Askarkar, S., and Singh, K. K. (2015). A comprehensive review on dry eye diseases: diagnosis, medical management, recent developments, and future challenges, Adv. Pharm., article ID 704946, pp 1-12.
2. Bonanno, J. A. (2012). Molecular mechanisms underlying the corneal endothelial pump, Exp. Eye Res., 95:2-7.
3. Klyce, S. D., and Crosson, C. E. (2009). Transport processes across the rabbit corneal epithelium: A review, Curr. Eye Res., 4:323-331.
4. Fullord, R. J., and Tucker, D. L. (1991). Changes in human tear protein levels with progressively increasing stimulus, Inves. Ophthalmol. Vis. Sci., 32(8):2290-2301.
5. Stuchell, R. N., Feldman, J. J., Forris, R. L., and Mondel, I. D. (1984). The effect of collection technique on tear composition, Inves. Ophthalmol. Vis. Sci., 25:374-377.
6. Sia, R. K., Ryan, D. S., Howard, R. S., Haymes, S., Zhou, Y., Coe, C. D., and Bower, K. S. (2016). Non-stimulated tear sample collection using polyvinyl alcohol (PVA) foam and polyester wick, Int. J. Ophthal. Clin. Res., 3:048-1/5.
7. Tomilinson, A., Khanai, S., Ramaesb, K., Diaper, C., and McFadyen, A. (2006). Tear film osmolarity: determination of a referent for dry eye diagnosis, Invest. Oph. Vis. Sci., 47:4309-4315.
8. Versura, P., and Campos, E. C. (2014). TearLab osmolarity system for diagnosing dry eye, Exp. Rev. Molec. Diagn., 13:119-129.
9. Yoon, D., Gadaria-Rathod, N., Oh, C., and Asbell, P. A. (2014). Precision and accuracy of TearLab osmometer in measuring osmolarity of salt solutions, Curr. Eye Res., 39:1247-1250.
10. Fleiszig, S. M. J. (2006). The pathogenesis of contact lens-related keratitis, Opto. Vis. Sci., 83:E866-E873.
11. Weissman, B. A. And Mondino, B. J. (2002). Risk factors for contact lens associated with microbial keratitis, Con. Lens Ant. Eye., 25:3-9.
12. Dart, J. K. G., Radford, C. F., Minassian, D., Verma, S., and Stapleton, F. (2008). Risk factors for microbial keratitis with contemporary contact lenses, Ophthal., 115:1647-1654.
13. Stapleton, F., Leay, L., Edwards, K., Naduvilath, T., Dart, J. K. G., Grian, G., and Holden, B. A. (2008). The incidence of contact-lens related microbial keratitis in Australia, Ophthal., 115:1655-1662.
14. Eter, N., and Gobbels, M. (2002). A new technique for tear film fluorometry, Br. J. Ophthal., 86:616-619.
15. Xu, K. P., and Tsubota, K. (1995). Correlation of tear clearance rate and fluorophotometric assessment of tear turnover, Br. J. Opthal., 79:1042-1045.
16. Senchyna, M., and Wax, M. B. (2008). Quantitative assessment of tear production: A review of methods and utility in dry eye drug discovery, J. Ocul. Biol. Dis. Inform., 1:1-6.
17. Wilson, S. A., and Last, A. (2004). Management of corneal abrasions, Am. Fam. Phys., 70:123-128.
18. Marquart, M. E., and Callaghan, R. J. (2013). Infectious keratitis: secreted bacterial proteins that mediate corneal damage, J. Opthal., 2013:369094-1/9.
19. Carni, N., Samarawickrama, C., White, A., and Stapleton, F. (2017). The diagnosis and management of contact lens-related microbial keratitis, Clin. Exp. Opto., 100:482-493.
20. Cornea treatment options in Delhi (2019). Cornea surgery in Delhi. eye7.in/cornea
21. Fleiszig, S. M. J., Kwong, M. S. F, and Evans, D. J. (2003). Modification of Pseudomonas aeeruginosa interactions with corneal epithelial cells by human tear fluid, Inf. Immun., 71:3866-3874.
22. Chalmers, R. L., and Begley, C. G. (2006). Dryness symptoms among an unselected clinical population with and without contact lens wear, Con. Lens Ant. Eye., 29:25-30.
23. Garcia-Porta, N., Rico-del-Viejo, L., Martin-Gil, A., Carracedo, G., Pintor, J., and Meijome, J. M. G. (2016). Differences in dry eye questionnaire symptoms in two different modalities for contact lens wear: Silicone-hydrogel in daily wear basis and overnight orthokeratology, BioMed Res. Intl., 2106:1-9.
24. Bron, A. J., Tomlinson, A., Foulks, G. N., Pepose, J. S., Baudouin, C., Geerling, G., Nichols, K. K., and Lemp, M. A. (2014). Rethinking dry eye disease: a perspective on clinical implications, Ocular Sur., 12:S1-S31.
25. Phadatare, S. P., Momin, M., Nighojkar, P., Askarkar, S., and Singh, K. K. (2015). A comprehensive review on dry eye diseases: diagnosis, medical management, recent developments, and future challenges, Adv. Pharmac., article ID 704946, pp 1-12.
26. Wolffsohn, J. S., et al. (2021). Clear evidence based contact lens practice, Con Lens Ant. Eye., 44:368-397.
27. Szczotka-Flynn, L., Ahearn, D. G., Barr, J., Benjamin, W., Kiang, T., Nichols, J. J., Schein, O. D., Stone, R. P., and Winterton, L. (2013). History, evolution, and evolving standards of contact lens care, Con. Lens Ant. Eye., 36:S4-S8.
28. Cox, M. E., and Dunn, B. (1986). Oxygen diffusion in poly(dimethl siloxane) using fluorescence quenching. I. Measurement technique and analysis, J. Poly. Sci. A: Polymer Chem., 24:621-636.
29. Muller, B. J., Burger, T., Borisov, S. M., and Slimant, I. (2015). High performance optical trace oxygen sensors based on NIR-emitting benzoporphyrins covalently coupled to silicone matrixes, Sen. Act. B: Chem., 216:527-534.
30. Mann, A., and Tighe, B. (2013). Contact lens interactions with the tear film, Exp. Eye Res., 117:88-98.
31. Brievogel, S. (2002). A primer on contact lens polymers, Eyewitness 2^{nd}quarter 32-35.
32. Santos, L., Rodrigues, D., Lira, M., Elisabete, M., oliverira, R., Oliveira, R., Vilar, E. Y. P., and Azeredo, J. (2007). The influences of surface treatment on hydrophobicity, protein adsorption and microbial colonization of silicone hydrogel contact lens, Con. Lens Ant. Eye., 30:183-188.
33. Smart Silicone Chemistry (2021). coopervision.com
34. coopervision.com/practitioner/our-products/contact-lens-technology/smart-silicone-chemistry
35. Osborn, G., and Dumbleton, K. (2019). Eye care professionals' perceptions of the benefits of daily disposable silicone hydrogel contact lenses, Con. Lens Ant. Eye., 42:373-379.
36. Sapkota, K., Franco, S., and Lira, M. (2018). Daily versus monthly disposable contact lens: Which is better for ocular surface physiology and comfort? Con. Lens Ant. Eye, 41:253-257.
37. Austin, D. T. R., and Hills, B. P. (2009). Two-dimensional NMR relaxation study of the pore structure in silicone hydrogel contact lenses, Appl. Mang. Reson., 35:581-591.
38. Guryca, V., Hobzova, R., Pradny, M., Sirc, J., and Michalek, J. (2007). Surface morphology of contact lenses probes with microscopy techniques, Con. Lens Ant. Eye, 30:215-222.
39. Al-Mujaini, A., Al-Kharusi, N., Thakral, A., and Wali, U. K. (2009). Bacterial keratitis: perspective on epidemiology, clinico-pathogenesis, diagnosis and treatment, Sultan Qab. Univ. Med. J., 9:184-195.
40. Collier, S. A., Gronostaj, M. P., MacGurn, A. K., Cope, J. R., Awsumb, K. L., Yoder, J. S., and Beach, M. J. (2010). Estimated burden of keratitis, United States, 2010, CDC, 63:1027-1030.
41. Efron, N. (2020). Twenty years of silicone hydrogel contact lenses: a personal perspective, Clin. Exp. Opt. 103:251-253.
42. Silicone hydrogels—What are they and how should they be used in everyday practice? Bausch&Lomb, CIBA Vision, Contact Lens Monthly, No. 5726, vol. 218.
43. Gromacki, S. J. (2013). Compliance with daily disposable contact lenses, Contact Lens Spectrum/Special Edition, p. 13.
44. Toshida, H., Takahashi, K., Sado, K., Kanai, A., and Murakami, A. (2008). Bifocal contact lenses: history, types, characteristics, and actual state and problems, Clin. Ophthal., 2:869-877.
45. Radford, C. F., Bacon, A. S., Dart, J. K. G., and Minassian, D. C. (1995). Risk factors for acanthamoeba keratitis in contact lens users: a case control study, Br. Med. J., 310:1567-1570.
46. Moore, M. B., McCulley, J. P., Luckenbach, M., Gelender, H., Newton, C., McDonald, M. B., and Visvesvara, G. S. (1985). Acanthamoeba keratitis associated with soft contact lenses, Am. J. Ophthal., 100:396-403.
47. Stapleton, F., Keay, L, Edwards, K., Naduvilath, T., Dart, J. K. G., Brian, G., Holden, B. A. (2008). The incidence of contact lens—related keratitis in Australia, Ophthal., 115:1655-1662.
48. Schein, O. D., Ormerod, L. D., Barraquer, E., Alfonso, E., Egan, K. M., Paton, B. G., and Kenyon, K. R. (1989). Microbiology of contact lens-related keratitis, Cornea., 8:281-285.
49. Poggio, E. C., Glynn, R. J., Schein, O. D., Seddon, J. M., Shannon, M. J., Scardino, V. A., and Kenyon, K. R. (1989). The incidence of ulcerative keratitis among users of daily-wear and extended wear soft contact lenses, N. Eng. J. Med., 321:779-783.
50. Orsborn, G., and Dumbleton, K. (2019). Eye care professionals perceptions of the benefits of daily disposable silicone hydrogel contact lenses, Con. Lens Ant. Eye, 42:373-379.
51. Stapleton, F., Edwards, K., Keay, L., Naduvilath, T., Dart, J. K. G., Brian, G., and Holden, B. (2012). Risk factors for moderate and severe microbial keratitis in daily wear contact lens users, Opthalmology., 119:1516-1521.
52. Weber, G., and Farris, F. J. (1979). Synthesis and spectral properties of a hydrophobic fluorescent probe: 6-propionyl-2-(dimethylamino)-naphthalene, Biochem., 18:3075-3078.
53. He, T., Wang, Y., Tian, X., Gao, Y., Zhao, X., Grimsdale, A. C., Lin, X., and Sun, H. (2016). An organic dye with very large Stokes-shift and broad tunability of fluorescence: Potential two-photon probe for bioimaging and ultra-sensitive solid-state gas sensor, Appl. Phys. Letts., 108:011901-1/5.
54. Stryer, L. (1965). The interaction of a naphthalene dye with apomyoglobin and apohemoglobin: A fluorescent probe of non-polar binding sites, J Mol. Biol., 13:482-495.
55. Badugu, R., Szmacinski, H., Reece, E. A., Jeng, B. H., and Lakowicz, J. R. (2020). Fluorescent contact lens for continuous non-invasive measurements of sodium and chloride ion concentrations in tears, Anal. Biochem., 608:113902.
56. Badugu, R., Reece, E. A., and Lakowicz, J. R. (2018). Glucose-sensitive silicone hydrogel contact lens toward tear glucose monitoring, J. Biomed. Optics., 23:057005/1.
57. Pawley, J. B. (1995). Handbook of Biological Confocal Microscopy, 2^{nd} ed. Plenum Press, New York, 632 pp.
58. Yavin, E., and Yavin, Z. (1974). Attachment and culture of dissociated cells from rat embryo cerebral hemispheres on polylysine-coated surface, J. Cell Biol., 62:540-546.
59. Mazia, D., Schatten, G., and Sale, W. (1975). Adhesion of cells to surfaces coated with polylysine, J. Cell Biol., 66:198-200.
60. Ye, R., Xu, H., Wan, C., Peng, S., Wang, L., Xu, H., Aguilar, Z. P., Xiong, Y., Zeng, Z., and Wei, H. (2013). Antibacterial activity and mechanism of action of e-poly-L-lysine, Biochem. Biophys. Res. Commun., 439; 148-153.
61. Mecke, A., Majoros, I. J., Patri, A. K., Baker, J. R., Holl, M. M. B., and On, B. G. (2005). Lipid bilayer disruption by polycationic polymers: The roles of size and chemical functional group, Langmuir, 21:10348-10354.
62. Crombez, L., Aldrian-Herrada, G., Konate, K., Nguyen, Q. N., McMaster, G. K., Brasseur, R., Heitz, F., and Divita, G. (2009) A new potent secondary amphiphatic cell-penetrating peptide for siRNA delivery into mammalian cells, Mol. Ther., 17:95-103.
63. Hallbrink, M., Floren, A., Elmquist, A., Pooga, M., Barfai, T., and Langel, U. (2001). Cargo delivery kinetics of cell-penetrating peptides, Biochim. Et Biophy. Acta, 1515:101-109.
64. Richard, J. P., Melikov, K., Vives, E., Ramos, C., Verbeure, B., Gait, M. J., Chernomordik, L. V., and Lebleu, B. (2003). Cell-penetrating peptides, J. Biol. Chem., 278:585-590.
65. Lakowicz, J. R., Shen, Y., D'Auria, S., Malicka, J., Fang, J., Gryczynski, Z., and Gryczynski, I. (2002). Radiative decay engineering, Anal. Biochem., 301:261-277.
66. Luensmann, D., and Jones, L. (2012). Protein deposition on contact lenses: The past, the present, and the future, Cont. Lens Ant. Eye., 35:53-64.
67. Luensmann, D., Zhang, F., Subbaraman, L., Sheardown, H., and Jones, L. (2009). Localization of lysozyme sorption to conventional and silicone hydrogel contact lenses using confocal microscopy, Curr. Eye Res., 34:683-697.
68. Badugu, R., Jeng, B. H., Reece, E. A., and Lakowicz, J. R. (2018). Contact lens to measure individual ion concentrations in tears and applications to dry eye disease, Anal. Biochem., 542:84-94.
69. Yang, Y., Lowry, M., Xu, X., Excobedo, J. O., Sibrian-Vazquez, M., Wong, L., Schowalter, C. M., Jensen, T. J., Fronczek, F. R., Warner, I. M., and Strongin, R. M. (2008). Seminaphthofluorones are a family of water soluble, low molecular weight, NIR-emitting fluorophores, Proc. Natl. Acad. Sci., 105:8829-8834.
70. Boens, N., Qin, W., and Basaric, N. (2006). Photophysics of the fluorescent pH indicator BCECF, J. Phys. Chem. A., 220:9634-9643.
71. Weiner, I. D., and Hamm, L. L. (1989). Use of fluorescent dye BCECF to measure intracellular pH in cortical collecting tubule, Am. J. Physiol., 256:F957-F964.
72. Alvarez-Leefmans, F. J., Herrera-Perez, J. J., Marquez, M. S. and Blanco, V. M. (2006). Simultaneous measurement of water volume and pH in single cells using BCECF and fluorescence imaging microscopy, Biophys. J., 90:608-618.
73. Hille, C., Berg, M., Bressel, L., Munzke, D., Primus, P., Lohmannstroben, H-G., and Dosche, C. (2008). Time-domain fluorescence lifetime imaging for intracellular pH sensing in living tissues, Anal. Bioanal. Chem., 391:1871-1879.
74. Szmacinski, H. S. and Lakowicz, J. R. (1993). Optical measurements of pH using fluorescence lifetimes and phase-modulation fluorometry, Anal. Chem., 65:1668-1674.
75. Tsien, R. Y. (1989). Fluorescent indicators of ion concentrations, Methods Cell Biol., 30:127-156.
76. Lamshanova, O., Mariot, P., Kyi, V. L., and Prevarskaya, N. (2016). Comparison of fluorescence probes for intracellular sodium imaging in prostate cancer cell lines, Eur. Biophys. J., 45:765-777.
77. Lamy, C. M., and Chatton, J-Y. (2011). Optical probing of sodium dynamics in neurons and astrocytes, Neuroimage, 58:572-578.
78. Schreiner, A. E., and Rose, C. R. (2012). Quantitative imaging of intracellular sodium, Curr. Micro. Cont. Adv. Sci. Tech., 119-129.
79. Martin, V. V., Rothe, A., and Gee, K. R. (2005). Fluorescent metal ion indicators based on the sodium indicator sodium binding benzofuranIsophthalate in HeLA cells, Anal. Biochem., 280:227-241.
80. Meier, S. D., Kovalchuk, Y., and Rose, C. R. (2006). Properties of the new fluorescent Na+ indicator CoroNa Green: Comparison with SBFI and confocal Na+ imaging, J. Neursci. Meth., 155:251-259.
81. Periasamy, A., and Clegg, R. M. (Ed.) (2009). FLIM Microscopy in Biology and Medicine, Chapman and Hall, 472 pp.
82. Meyer, J., Untiet, V., Fahlke, C., Gensch, T., and Rose, C. R. (2019). Quantitative determination of cellular [Na⁺] by fluorescence lifetime imaging with CoroNaGreen, J. Gen. Phys., 151:1319-1331.
83. Naumann, G., Lippmann, K., and Eilers, J. (2018). Photophysical properties of Na⁺ indicator dyes suitable for quantitative two-photon fluorescence lifetime measurements, J. Micro., 272:136-144.
84. Whitaker, J. E., Haugland, R. P., and Prendergast, F. G. (1991). Spectral and photophysical studies of benzo[c]xanthene dyes: dual emission pH sensors, Anal. Biochem., 194:330-344.
85. Despa, S., Vecer, J., Steels, P., and Ameloot, M. (2000). Fluorescence lifetime microscopy of the Na indicator sodium green in HeLa cells, Anal. Biochem., 281:159-175.
86. Yin, J., Hu, Y., and Yoon, J. (2015). Fluorescent probes and bioimaging: alkali metals, alkaline earth metals and pH, Chem. Soc. Rev., 44:4619-4644.
87. Thermo Fisher Scientific (201). Molecular Probes Handbook., Introduction to Fluorescence Techniques, Chap. 21. Pp. 907-908.
88. Ward, D. T. (2004). Calcium receptor-mediated intracellular signalling, Cell Calcium, 35:217-228.
89. Song, Z., Wang, Y., Zhang, F., Yao, F., and Sun, C. (2019). Calcium signalling pathways: Key pathways in the regulation of obesity, Int. J. Mol. Sci., 20:2768.
90. Grynkiewicz, G., Poenie, M., and Tsien, R. Y. (1985). A new generation of Ca²⁺ indicators with greatly improved fluorescent properties, J. Biol. Chem., 260(6):3440-3450.
91. Tsien, R. Y. (1989). Fluorescent indicators of ion concentrations, Methods Cell Biol., 30:127-156.
92. Molecular Probes (2010). The Molecular Probes Handbook, 11^{th} ed. 1060 pp.
93. AAT Bioquest, Inc., Sunnyvale, Calif. 94085
94. Lakowicz, J. R., Szmacinski, H., and Johnson, M. L. (1992). Calcium imaging using fluorescence lifetimes and long-wavelength probes, J. Fluoresc., 2(1):47-62.
95. Szmacinski, H., and Lakowicz, J. R. (1996). Fluorescence lifetime characterization of magnesium probes: Improvement of Mg²⁺ dynamic range and sensitivity using phase-modulation fluorometry, J. Fluoresc., 6:83-95.
96. Doussineau, T., Schulz, A., Lapresta-Fernandez, A., Moro, A., Korsten, S., Trupp, S., and Mohr, G. J. (2010). Fluorescent ratiometric nanosensors, Chem. Eur. J., 16:10290-10299.
97. Park, S-H., Kwon, N., Lee, J-H., Yoon, J., and Shin, I. (2020). Synthetic ratiometric fluorescent probes for detection of ions, Chem. Soc. Rev., 49:143-179.
98. Jayaraman, S., Teitler, L., Skalski, B., and Verkman, A. S. (1999). Long-wavelength iodide-sensitive fluorescent indicators for measurement of functional CFTR expression in cells, Am. J. Physiol., 277:C1008-C1018.
99. Szmacinski, H., and Lakowicz, J. R. (1996). Frequency-domain lifetime measurements and sensing in highly scattering media, Sen. Actua B., 30:207-215.
100. Gamal, A. El., and Eltoukhy, H. (2005). CMOS image sensors, IEEE Cir. Dev. Mag., 21(3):8755-3996.
101. Hain, R., Kahler, C. J., and Tropea, C. (2007). Comparison of CCD, CMOS and intensified cameras, Exp. Fluids, 42(3):403-411.
102. Janesick, J. and Putnam, G. (2003). Developments and applications of high performance CCD and CMOS imaging arrays, Annu. Rev. Nuc. Particle Sci., 53:263-300.
103. Rossun, E. R. (1995). CMOS image sensors: Electronic camera on a chip, IEEE Trans. Elec. Dev., 44(10):1689-1698.
104. Moynihan, T. (2011). CMOS is winning the camera sensor battle, and here's why, techhive.com/article/246931/cmos is winning the camera sensor battle and heres why.html
105. Gu, Y. Comparing sCMOS, and/or.com/learning-academy/comparing-scmos-compare-sCMOS-with-other-detectors, Andor Instrument Company, 1/4.
106. Mattioli Della Rocca, F., Nedbal, J., Tyndall, D., Krstajic, N., Day-Uei Li, D., Ameer-Beg, S. M., and Henderson, R. K. (2016). Real-time fluorescence lifetime actuation for cell sorting using a CMOS SPAD silicon photomultiplier, Optics Letts., 41(4):673-676.
107. Rae, B. R., Muir, K. R., Gong, Z., McKendry, J., Girkin, J. M., Gu, E., Renshaw, D., Dawson, M. D., and Henderson, R. K. (2009). A CMOS time-resolved fluorescence lifetime analysis micro-system, Sensors, 9:9255-9274.
108. Li, D. D-U., Arit, J., Tyndall, D., Walker, R., Richardson, J., Stoppa, D., Charbon, E., and Henderson, R. K. (2011). Video-rate fluorescence lifetime imaging camera with CMOS single-photon avalanche diode arrays and high-speed imaging algorithm, J. Biomed. Optics., 16(9):096012-1/12.
109. en.wikipedia.org/wiki/Tango_(platform)
110. lenovo.com/us/en/smart-devices/-lenovo-smartphones/phab-series/c/phab-series
111. Badugu, R., Szmacinski, H., Reece, E. A., and Lakowicz, J. R. (2021). Sodium-sensitive contact lens for diagnostics of ocular pathologies, Sen Actua B., 331:129434.

1. A probe composition comprising:
a hydrophobic portion;
a hydrophilic portion;
an analyte-binding portion configured to bind to an analyte in an aqueous solution; and
a fluorophore portion configured to change an optical property of fluorescent light emitted in response to incident excitation light when the composition changes between a first state in which the analyte is not bound to the analyte-binding portion and a second state in which the analyte binds to the analyte-binding portion.

2. A probe composition as recited in claim 1, wherein the optical property of the emitted fluorescent light is selected from a group consisting of intensity, ratio of intensity among a plurality of frequencies, lifetime of emission, and phase difference from the incident excitation light.

3. A probe composition as recited in claim 1, further comprising a spacer portion configured to place the analyte-binding portion in an aqueous solution when the hydrophobic portion is attracted to an interface with a hydrophobic structure and to place the fluorophore within a certain distance of the analyte-binding portion such that the fluorophore portion is affected by binding of the analyte to the analyte-binding portion.

4. A probe composition as recited in claim 1, wherein the fluorophore portion includes an electron donor sub-portion and a separate electron acceptor sub-portion.

5. A probe composition as recited in claim 1, wherein the fluorophore portion includes an electron donor sub-portion and a separate acceptor sub-portion, both sub-portions involved in Forster resonance energy transfer (FRET).

6. The probe composition as recited in claim 4 which is a modular composition wherein the donor sub-portion and the acceptor sub-portion are separate moieties connected by an aliphatic linker, the linker including a diboronic acid.

7. The probe composition as recited in claim 1, having the structural formula; 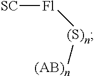
wherein Fl is the fluorophore portion;
AB is the analyte binding portion;
SC is the hydrophobic portion, the hydrophobic portion comprising a C₈-C1₈ alkyl group;
S is a group that provides sufficient spacing between AB and Fl such that when AB binds the analyte, the fluorophore portion changes an optical property of fluorescent light emitted in response to incident excitation light when the probe composition changes between a first state in which the analyte is not bound to the analyte-binding portion and a second state in which the analyte binds to the analyte-binding portion;
wherein n represents an integer from 1 to 20; and
wherein the fluorophore portion changes an optical property of fluorescent light emitted in response to incident excitation light when the composition changes between a first state in which the analyte is not bound to the analyte-binding portion and a second state in which the analyte binds to the analyte-binding portion via a photophysical mechanism selected from the group: quenching, photo-induced electron transfer (PET), and intramolecular charge transfer (ICT).

8. The probe composition as recited in claim 7, wherein SC further comprises separating units of polyethelene glycol, hydroxyl groups, or arginine peptide.

9. The probe composition as recited in claim 7, wherein the analyte binding portion comprises boronic acid or a diboronic acid.

10. The probe composition as recited in claim 7, wherein the analyte to be bound by the analyte binding portion is selected from a group comprising: glucose, cations of Group I and Group II metals, and anions of Group VIIA.

11. A material comprising:
a silicone hydrogel substrate having a hydrogel network that allows flow of aqueous solution through the hydrogel network, wherein a silicone network occupies interstices of the hydrogel network; and
the probe composition of claim 1, wherein the hydrophobic portion of the probe composition is attracted to an interface between the hydrogel network and the silicone network.

12. A material as recited in claim 11, further comprising a treated surface of the material, wherein the treated surface has stronger hydrophobic attraction than an untreated surface of the material whereby the concentration of the probe composition is greater on the treated surface of the material than on an untreated surface of the material or internal to the material.

13. A material as recited in claim 11, wherein the material is incorporated into a contact lens.

14. A material as recited in claim 11, wherein the probe composition is a modular composition
wherein the donor sub-portion and the acceptor sub-portion are separate species connected by an aliphatic linker, the aliphatic linker including a diboronic acid; and
wherein the separate donor sub-portion and the separate acceptor sub-portion are a pair of separate species selected from a group of pairs consisting of:
Quinolinium C-18 paired with 7-nitrobenz-2-oxa-1,3-diazol-4-yl (NBD) with a C18 side chain (NBD C-18);
Naphthalene paired with Dansyl;
Dansyl paired with fluorescein-5-isothiocyanate (FITC);
Dansyl paired with octadecylrhodamine (ODR);
1-N6-ethenoadenosine (ε-A) paired with NBD; IAF paired with tetramethylrhodamin (TMR);
Pyrene paired with coumarin;
FITC paired with TMR;
5-(2-((iodocetyl)amino)ethyl)amino)naphthalene-1-sulfonic acid (IAEDANS) paired with FITC;
IAEDANS paired with 5-iodoacetamidofluorescein (IAF);
IAF paired with an enzyme immunoassay (EIA);
carboxylfluorescein, succinimidyl ester (CF) paired with Texas Red (TR);
4,4-difluoro-4-bora-3a,4a-diaza-s-indacene (Bodipy) paired with Bodipy;
B-phycoerythrin (BPE) paired with a cyanine dye (Cy);
Terbium paired with Rhodamine;
Europium paired with Cy; and
Europium paired with allophycocyanin (APC).

15. The material as in claim 14, wherein the acceptor sub-portion further comprises one or a plurality of halogen groups.

16. The material as recited in claim 11, wherein the probe composition has a longitudinal axis length between 2-8 nm when the analyte is not bound to the analyte binding portion.

17. The material as recited in claim 11, wherein the probe composition has the structural formula: 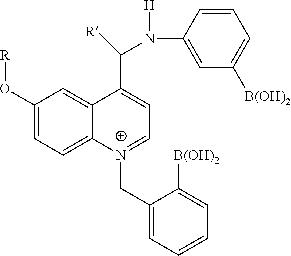
wherein R is one of the species selected from the group consisting of:
a C₈-C₁₈ alkyl group; 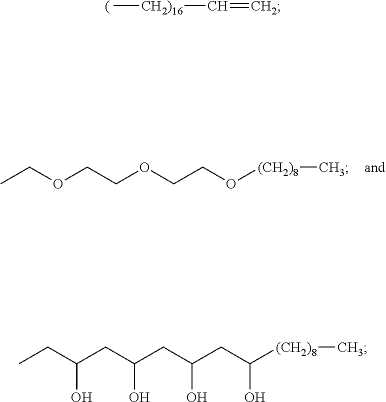
and
wherein R′ is hydrogen or a ketone functional group.

18. The material as recited in claim 11, wherein the probe composition has the structural formula: 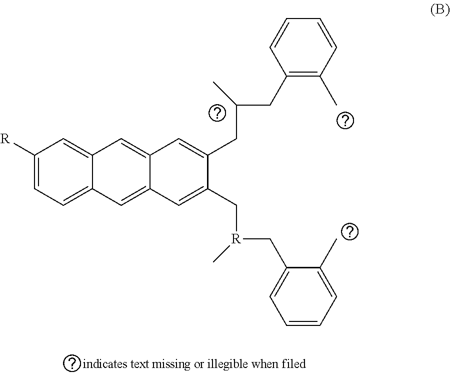
wherein R is one of the species selected from the group comprising:
a C₈-C₁₈ alkyl group;

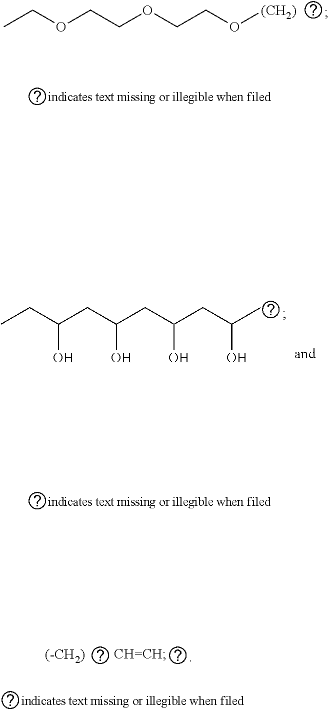

19. The material as recited in claim 11, wherein the probe composition has the structural formula: 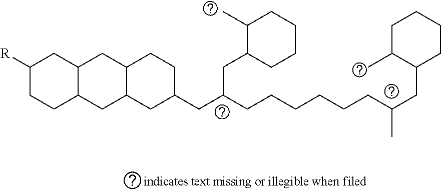
wherein R is one of the species selected from the group comprising:
a C₈-C₁₈ alkyl group;

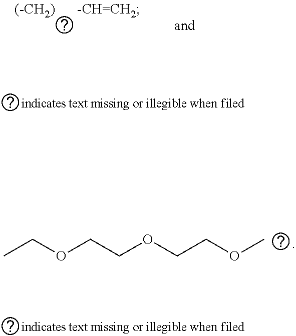

20. The material as recited in claim 11, wherein the probe composition has the structural formula: 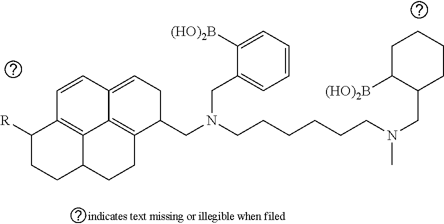
wherein R is one of the species selected from the group comprising:
a C₈-C₁₈ alkyl group

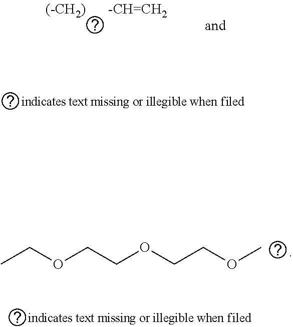

21. The material as recited in claim 11, wherein the probe composition has the structural formula: 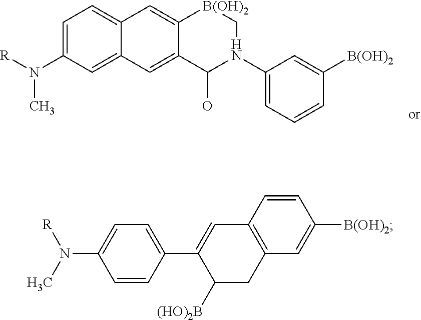
wherein R is one of the species selected from the group consisting of:
a C₈-C₁₈ alkyl group;

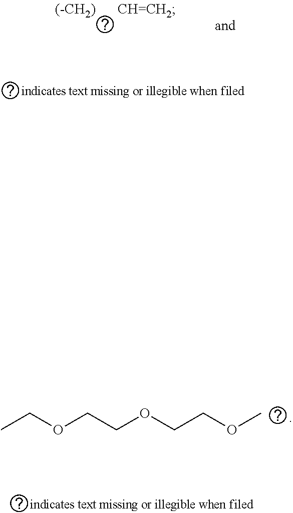

22. The material as recited in claim 11, wherein the probe composition has the structural formula: 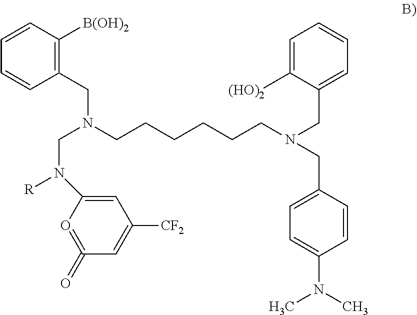
wherein R is one of the species selected from the group comprising:
a C₁-C₈ alkyl group;

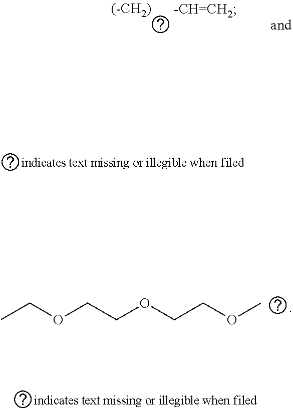

23. The material as recited in claim 11, wherein the probe composition has the structural formula: 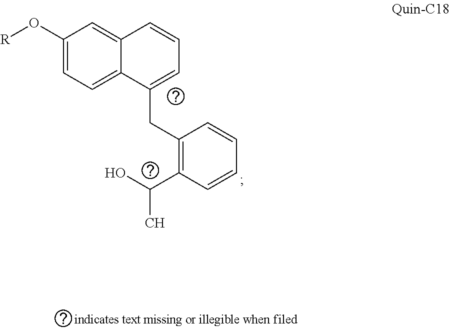
wherein R is one of the species selected from the group comprising:
one or a plurality of C₈-C₁₈ alkyl groups

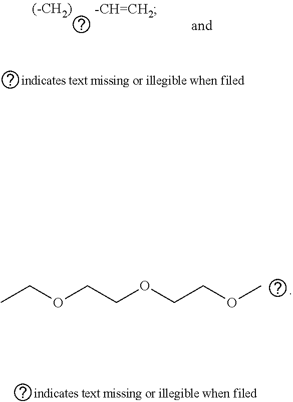

24. The material as recited in claim 11 configured to detect pH levels wherein the probe composition is a quinolinium based probe composition having a hydrophobic side chain comprising between 8-18 carbon atoms.

25. The material as recited in claim 11 configured to detect ion concentrations of at least one Group I, Group II, or Group VIIA element.

26. The material as recited in claim 25 wherein the probe composition is configured to detect cations of Group I metals and wherein the probe composition has the structural formula selected from the group consisting of:

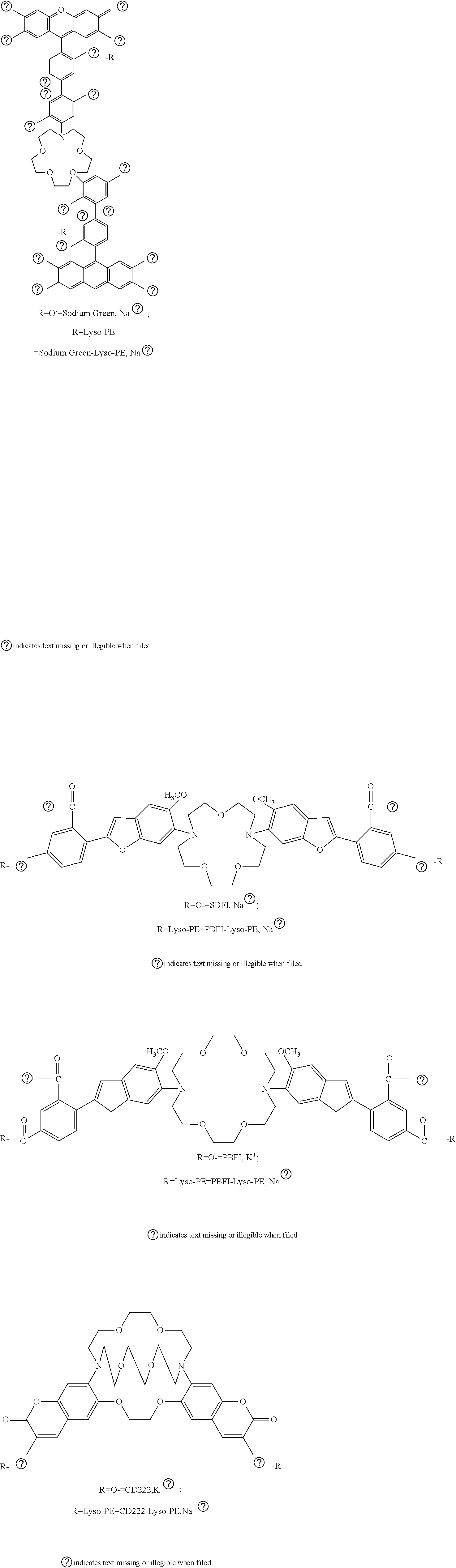

27. The material as recited in claim 25 wherein the probe composition is configured to detect cations of Group II metals, and wherein the probe composition has the structural formula selected from the group consisting of:

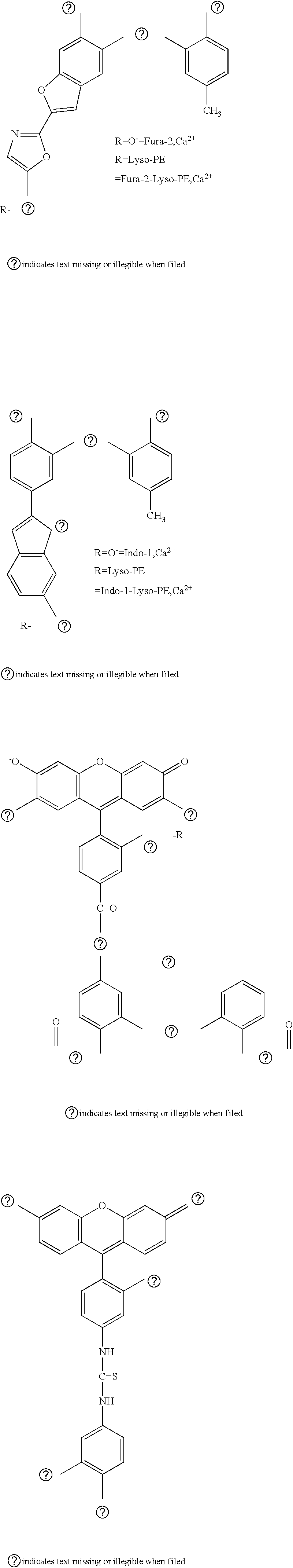

28. The material as recited in claim 25 wherein the probe composition is configured to detect an anion of a Group VIIA element and wherein the probe composition has the structural formula selected from the group consisting of:

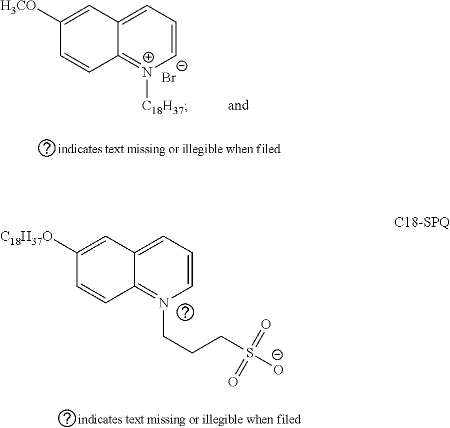

29. A system comprising:
the material of claim 11; and
a remote monitor subsystem configured to detect the change of the optical property of the fluorescent light emitted in response to the incident excitation light without mechanically contacting the material.

30. A system as recited in claim 29 wherein the material is fixed to a microfluidic device.

31. A system as recited in claim 29 wherein the material is incorporated into a contact lens.

32. A system as recited in claim 29, wherein the monitor subsystem further comprises:
an incident light source;
a light detector; and
a processing system, the processing system further comprising at least one processor; and at least one memory including one or more sequences of instructions, the at least one memory and the one or more sequences of instructions configured to, with the at least one processor, cause the system to perform at least the following, operate the incident light source to illuminate the material, operate the light detector to obtain data that indicates the property of the emitted fluorescent light, and determine a concentration of the analyte based on the data that indicates the property of the emitted fluorescent light.

33. A system as recited in claim 32 wherein:
the system further comprising an analyte response device; and
the at least one memory and the one or more sequences of instructions are further configured to, with the at least one processor, cause the system to operate the analyte response device based on the concentration of the analyte.

34. A method comprising:
obtaining a silicone hydrogel substrate;
contacting the substrate with an aqueous solution that comprises the probe composition as recited in claim 1, wherein the composition is a probe, to form a probe-substrate material;
contacting probe-substrate material with an aqueous sample solution;
illuminating, using a light source, the probe-substrate material in contact with the sample solution;
measuring a value of a property of the fluorescent light emitted by the material in contact with the sample solution in response to the illuminating; and
determining a value of a concentration of the analyte in the aqueous sample solution based on the value of the property.

35. A method as recited in claim 34, wherein said step of determining the concentration of the analyte is performed automatically on a processor.

36. A method as recited in claim 34, further comprising operating an analyte response device based on the value of the concentration of the analyte in the aqueous sample solution.

37. A non-transitory computer-readable medium carrying one or more sequences of instructions, wherein execution of the one or more sequences of instructions by one or more processors causes the one or more processors to perform the steps of:
operating an incident light source to illuminate the material of claim 11,
operating a light detector to obtain data that indicates the property of fluorescent light emitted in response to operating the incident light source, and
determining a concentration of the analyte based on the data that indicates the property of the emitted fluorescent light.

38. A computer-readable medium as recited in claim 37, wherein execution of the one or more sequences of instructions by one or more processors further causes the one or more processors to perform the step of operating an analyte response device based on the concentration of the analyte.
